SEQUENCE LISTING (231 sequences)

SEQ ID NO: 1 - DNA, 60 bp, synthetic construct
  source 1..60: mol_type = other DNA
ctgagtggtcagagatctgtaaagcatgactttcaaggatggttcttaggggactgtgta

SEQ ID NO: 2 - DNA, 60 bp, synthetic construct
  source 1..60: mol_type = other DNA
aggacttgaatgaggaaaccaacactttgagaaaccaaagtcctttttcccaaaggttct

SEQ ID NO: 3 - DNA, 60 bp, synthetic construct
  source 1..60: mol_type = other DNA
gccattaagatttggatgggaagttatgggtaatgagaatataatgacatcttgcaacat

SEQ ID NO: 4 - DNA, 60 bp, synthetic construct
  source 1..60: mol_type = other DNA
gatggcccagcctgtaagatactgtatatgcgctgctgtagataccggaatgaattttct

SEQ ID NO: 5 - DNA, 60 bp, synthetic construct
  source 1..60: mol_type = other DNA
ggcctcacattctgctctgctaagtttggagaaaacagaacaataaaccagatgcaggtg

SEQ ID NO: 6 - DNA, 60 bp, synthetic construct
  source 1..60: mol_type = other DNA
aagtactggaatgtaatggttgaaattcctattcagtgatctggaagaactctaatgttc

SEQ ID NO: 7 - DNA, 60 bp, synthetic construct
  source 1..60: mol_type = other DNA
ccaacgcacaccagtcttctcaatctgactgtaatctaatctgttgtgcttttgttggac

SEQ ID NO: 8 - DNA, 60 bp, synthetic construct
  source 1..60: mol_type = other DNA
ggtttaaagctgaagaggttgaagctaaaaggaaaaggttgttgttaatgaatatcaggc

SEQ ID NO: 9 - DNA, 60 bp, synthetic construct
  source 1..60: mol_type = other DNA
ggctaaaagggaaaaaggatatgtggagaatcatcaagatatgaattgaatcgctgcgat

SEQ ID NO: 10 - DNA, 60 bp, synthetic construct
  source 1..60: mol_type = other DNA
cctttcaaacatgatcaaagatttcccaatgtgatctcatcatcatggatactcaatttg

SEQ ID NO: 11 - DNA, 60 bp, synthetic construct
  source 1..60: mol_type = other DNA
ggggaacaatgagggcatttcatgaaccatctcaggcacttctgcatcacggaagacctg

SEQ ID NO: 12 - DNA, 60 bp, synthetic construct
  source 1..60: mol_type = other DNA
tgccttgagaatttcaaaagaggtaatcaggaaaagagagagagaaaaactacacgctgt

SEQ ID NO: 13 - DNA, 60 bp, synthetic construct
  source 1..60: mol_type = other DNA
gtctgggattaagggcaaatctattacttttgcaaactgtcctctacatcaattaacatc

SEQ ID NO: 14 - DNA, 60 bp, synthetic construct
  source 1..60: mol_type = other DNA
taaaaaagaaatagtcagtgttttcctcctttcaaccgagactatttctggattgtgtgc

SEQ ID NO: 15 - DNA, 60 bp, synthetic construct
  source 1..60: mol_type = other DNA
ttttcagaaagaagtctggaccaggctgaaggcatttgcaaagcttcccccaaatgtctt

SEQ ID NO: 16 - DNA, 60 bp, synthetic construct
  source 1..60: mol_type = other DNA
cctcattgccttattcggagtactattatccaatatatgaaatcaaagattgtctcctga

SEQ ID NO: 17 - DNA, 60 bp, synthetic construct
  source 1..60: mol_type = other DNA
tggcatcatacaaagagcaggagaagcaaacacccagaactcttttgctggtcagagatt

SEQ ID NO: 18 - DNA, 60 bp, synthetic construct
  source 1..60: mol_type = other DNA
tccagacctaccttgtacgcacatagacattttcatatgcactggatggagttagggaaa

SEQ ID NO: 19 - DNA, 60 bp, synthetic construct
  source 1..60: mol_type = other DNA
atctttgttaattattttggggagtagttgggaaatggaaaggtgaattggctctagagg

SEQ ID NO: 20 - DNA, 60 bp, synthetic construct
  source 1..60: mol_type = other DNA
gttcatttccagccctttctagatctgatcttttagggggaaagacagcttaaaatgttc

SEQ ID NO: 21 - DNA, 60 bp, synthetic construct
  source 1..60: mol_type = other DNA
tgaatgtcatgtttatgtcatagacgtagaaaacgcatccttgaattaaactgccttaac

SEQ ID NO: 22 - DNA, 60 bp, synthetic construct
  source 1..60: mol_type = other DNA
tactggagtaactgagtcgggacgctgaatctgaatccaccaataaataaagcttctgca

SEQ ID NO: 23 - DNA, 60 bp, synthetic construct
  source 1..60: mol_type = other DNA
cagattccccagaaactaccttttgcccaaagaacatgctcagtatttggggcatttcct

SEQ ID NO: 24 - DNA, 60 bp, synthetic construct
  source 1..60: mol_type = other DNA
aggcaggggtggtgattcatgctgtgtgactgactgtgggtaataaacacacctgtcccc

SEQ ID NO: 25 - DNA, 60 bp, synthetic construct
  source 1..60: mol_type = other DNA
tggatttctaaactgctcaattttgactcaaaggtgctatttaccaaacactctccctac

SEQ ID NO: 26 - DNA, 60 bp, synthetic construct
  source 1..60: mol_type = other DNA
ccaatccaacaactataggctgggttaaataaaaggtcattattgtctatattccaagtg

SEQ ID NO: 27 - DNA, 60 bp, synthetic construct
  source 1..60: mol_type = other DNA
tctaccacattaaattctccattacatctcactattggtaatggcttaagtgtaaagagc

SEQ ID NO: 28 - DNA, 60 bp, synthetic construct
  source 1..60: mol_type = other DNA
tgaggaattcttgtacgcagttttctttggctttacgagccgattaaaagaccgtgtgaa

SEQ ID NO: 29 - DNA, 60 bp, synthetic construct
  source 1..60: mol_type = other DNA
ctggtctttgaaagaaatgtactactaaagagcactagttgtgaatttagggtgttaaac

SEQ ID NO: 30 - DNA, 60 bp, synthetic construct
  source 1..60: mol_type = other DNA
atgatgggagagctctggcagatgtcccaatcctggaggtcatccattaggaattaaatt

SEQ ID NO: 31 - DNA, 60 bp, synthetic construct
  source 1..60: mol_type = other DNA
caacttgctctttcatatgagttggtcatagcatgtaagaaccaatcttgaaatatcgtt

SEQ ID NO: 32 - DNA, 60 bp, synthetic construct
  source 1..60: mol_type = other DNA
cctggatcagagtaagaatgtcttaagaagaggtttgtaaggtcttcataacaaagtggt

SEQ ID NO: 33 - DNA, 60 bp, synthetic construct
  source 1..60: mol_type = other DNA
ctcctggactgcttcttttggctctccgacaactccggccaataaacactttctgaattg

SEQ ID NO: 34 - DNA, 60 bp, synthetic construct
  source 1..60: mol_type = other DNA
aaccaacccataattgcattttacttgtcgtggttcgatctgattgtattgtcgaaggac

SEQ ID NO: 35 - DNA, 60 bp, synthetic construct
  source 1..60: mol_type = other DNA
attcctttatgagctctccatatccttcttgagaaactggttaaaaaaggaataggggta

SEQ ID NO: 36 - DNA, 60 bp, synthetic construct
  source 1..60: mol_type = other DNA
agtgggggttgtgtaaaggggaagtcatcttttgagatccagatagacatggtttgtgca

SEQ ID NO: 37 - DNA, 60 bp, synthetic construct
  source 1..60: mol_type = other DNA
tcagcttaagtacttattgtggtagtgagtcctacggtatttcagtaaaaaggaattcat

SEQ ID NO: 38 - DNA, 60 bp, synthetic construct
  source 1..60: mol_type = other DNA
ggcaagagttatcatagaacaacaaaatagagtggactcttttagagcatctatatctgc

SEQ ID NO: 39 - DNA, 60 bp, synthetic construct
  source 1..60: mol_type = other DNA
ggagtttctgtttagggcattaaaaattcccgcaaactataaagagcaatgttttcagtc

SEQ ID NO: 40 - DNA, 60 bp, synthetic construct
  source 1..60: mol_type = other DNA
ataattctctgtacaggggggtttgtgctatacactgggatgtctaattgcagcaataaa

SEQ ID NO: 41 - DNA, 60 bp, synthetic construct
  source 1..60: mol_type = other DNA
aggactttaatcttggtgatgccttggacagcagcaactccatgcaaaccatccaaaaga

SEQ ID NO: 42 - DNA, 60 bp, synthetic construct
  source 1..60: mol_type = other DNA
gggcaaaatgtatcactccaaacactactgattcagcattgttttcatgtcttaaaattg

SEQ ID NO: 43 - DNA, 60 bp, synthetic construct
  source 1..60: mol_type = other DNA
ctggatgtttagcttcttactgcaaaaacataagtaaaacagtcaactttaccatttccg

SEQ ID NO: 44 - DNA, 60 bp, synthetic construct
  source 1..60: mol_type = other DNA
ggtaacttgcaggaatattctattggaaaagataacaggaagtacaagtgcttcttgacc

SEQ ID NO: 45 - DNA, 60 bp, synthetic construct
  source 1..60: mol_type = other DNA
tcaatggttagcagaagggagaaaagaaagcaggaaaatgtgctattgagattccagtgg

SEQ ID NO: 46 - DNA, 60 bp, synthetic construct
  source 1..60: mol_type = other DNA
cctgggtttacaacgctgttaggaaaattaaccaatgaataaagcaacgttcagtgcgca

SEQ ID NO: 47 - DNA, 60 bp, synthetic construct
  source 1..60: mol_type = other DNA
tttttgtaccttgtcactataactacttcctagtcaaagaacgaaatgtaactgttaccg

SEQ ID NO: 48 - DNA, 60 bp, synthetic construct
  source 1..60: mol_type = other DNA
ttctagctgttattttgctatttggcatttacataaaagcacacgatgaagcaggtatcg

SEQ ID NO: 49 - DNA, 60 bp, synthetic construct
  source 1..60: mol_type = other DNA
ttgggtttatttccaggtcacagaattgctgttaacactagaaaacacacttcctgcacc

SEQ ID NO: 50 - DNA, 60 bp, synthetic construct
  source 1..60: mol_type = other DNA
gaacagctccttactctgaggaagttgattcttatttgatggtggtattgtgaccactga

SEQ ID NO: 51 - DNA, 60 bp, synthetic construct
  source 1..60: mol_type = other DNA
ctttcttatttactaagaatttgcctgtttgaataagaacaaaacgctaaggtgggtagc

SEQ ID NO: 52 - DNA, 60 bp, synthetic construct
  source 1..60: mol_type = other DNA
ctagagagcagaaataaaaagcatgactatttccaccatcaaatgctgtagaatgcttgg

SEQ ID NO: 53 - DNA, 60 bp, synthetic construct
  source 1..60: mol_type = other DNA
gttcaggggcatcacctactttgcttacttgattcaaggctctcattaaagacattttag

SEQ ID NO: 54 - DNA, 60 bp, synthetic construct
  source 1..60: mol_type = other DNA
gttggtagagggagtatgataaaatgtttaaatctcatttggttaccttgagtcctggaa

SEQ ID NO: 55 - DNA, 60 bp, synthetic construct
  source 1..60: mol_type = other DNA
aattcaacagtgtggaagctttaggggaacatggagaaagaaggagaccacataccccaa

SEQ ID NO: 56 - DNA, 60 bp, synthetic construct
  source 1..60: mol_type = other DNA
caagttgtgcaaagtgagaaagatctttgtgggcacaaaaggaatccctcatctggtgac

SEQ ID NO: 57 - DNA, 60 bp, synthetic construct
  source 1..60: mol_type = other DNA
caagagaacctggagaaaactaccgtattcaagagattaatcaaaatcagtgttttagcc

SEQ ID NO: 58 - DNA, 60 bp, synthetic construct
  source 1..60: mol_type = other DNA
ccgaatgaccttaaaggtgatcggctttaacgaatatgtttacatatgcatagcgctgca

SEQ ID NO: 59 - DNA, 60 bp, synthetic construct
  source 1..60: mol_type = other DNA
agtttatgggccagaatattctgtataccagacattggtaagctctcatggtttacagga

SEQ ID NO: 60 - DNA, 60 bp, synthetic construct
  source 1..60: mol_type = other DNA
ccatgtggccagtctaccatggggcccaggagttggggaaacacaataaaggtggcatac

SEQ ID NO: 61 - DNA, 60 bp, synthetic construct
  source 1..60: mol_type = other DNA
atgcttaaacccacggaagggggagactctttcggatttgtagggtgaaatggcaattat

SEQ ID NO: 62 - DNA, 60 bp, synthetic construct
  source 1..60: mol_type = other DNA
ttctttcttcaaagagtcatcagaataacatggattgaagagacttccgaacacttgcta

SEQ ID NO: 63 - DNA, 60 bp, synthetic construct
  source 1..60: mol_type = other DNA
tgaagtcagcgttaaccatgtgcatacaacttaaggaattttttcctcctcatgtaaatt

SEQ ID NO: 64 - DNA, 60 bp, synthetic construct
  source 1..60: mol_type = other DNA
gttaaacagggattatagtacttgtctcacaaagtttctgtgagaattaaacaaggggat

SEQ ID NO: 65 - DNA, 60 bp, synthetic construct
  source 1..60: mol_type = other DNA
cagcctgtgtgatacaagtttgatcccaggaacttgagttctaagcagtgctcgtgaaaa

SEQ ID NO: 66 - DNA, 60 bp, synthetic construct
  source 1..60: mol_type = other DNA
aatgcacagatctgcttgatcaattcccttgaatagggaagtaacatttgccttaaattt

SEQ ID NO: 67 - DNA, 60 bp, synthetic construct
  source 1..60: mol_type = other DNA
tttccaataaccacctaaattttaacaaaggttccttctaagtggtagaacttggggtgg

SEQ ID NO: 68 - DNA, 60 bp, synthetic construct
  source 1..60: mol_type = other DNA
agttatgcttcccttcatgttatatgcacattgccaagaattactgtcaagagaaatgat

SEQ ID NO: 69 - DNA, 60 bp, synthetic construct
  source 1..60: mol_type = other DNA
aaggtttgaaggttacggctcagggctgccccattaaagtcagtgttgtgttctaaaaaa

SEQ ID NO: 70 - DNA, 60 bp, synthetic construct
  source 1..60: mol_type = other DNA
ggactgtatgaatttatagaaaattgaatctaatttcagaagagcgcactgtcttctcag

SEQ ID NO: 71 - DNA, 60 bp, synthetic construct
  source 1..60: mol_type = other DNA
tacatttctttgggtttctagagacgcccctaagtcacctgcttcattagacggtttcca

SEQ ID NO: 72 - DNA, 60 bp, synthetic construct
  source 1..60: mol_type = other DNA
ggcctaattgagggaaggaggaaattcataccagcagttttcaaataaaagaattgttct

SEQ ID NO: 73 - DNA, 60 bp, synthetic construct
  source 1..60: mol_type = other DNA
tccaattctacactcagttaaagaccattacttctcagtggaaagaagaagatgctactc

SEQ ID NO: 74 - DNA, 60 bp, synthetic construct
  source 1..60: mol_type = other DNA
gtggggacttcgtgggaggcactcatggctctctgggtctaatgaataaagtcctccaca

SEQ ID NO: 75 - DNA, 60 bp, synthetic construct
  source 1..60: mol_type = other DNA
ccaggatcttaaggaagaatattctaggaagaaggaaactatttctactgctaataaagc

SEQ ID NO: 76 - DNA, 60 bp, synthetic construct
  source 1..60: mol_type = other DNA
agaaaacccttttctacagttagggttgagttacttcctatcaagccagtacgtgctaac

SEQ ID NO: 77 - DNA, 60 bp, synthetic construct
  source 1..60: mol_type = other DNA
tagggaatgaatgaatgaatatggattgctgttaactagaaacacttctgtatgtcagtc

SEQ ID NO: 78 - DNA, 60 bp, synthetic construct
  source 1..60: mol_type = other DNA
gtacttagctggaagaacatgttaattctgcaatatgtttcttggttaaacattgcacag

SEQ ID NO: 79 - DNA, 60 bp, synthetic construct
  source 1..60: mol_type = other DNA
actctcttaggtcatttttcaatgtgtgtaaccaaaagttaatcagaataaagcggaagc

SEQ ID NO: 80 - DNA, 60 bp, synthetic construct
  source 1..60: mol_type = other DNA
aatgctttgttggagtttaaaaattcagggaaaaaatcggcagaccattagttactatgg

SEQ ID NO: 81 - DNA, 60 bp, synthetic construct
  source 1..60: mol_type = other DNA
aagaaaccagcatgtgactttcctagataacactgctttctcataataaagactatttgc

SEQ ID NO: 82 - DNA, 60 bp, synthetic construct
  source 1..60: mol_type = other DNA
gttggcattgatatggtacaacctgcaaattacttgcagttctgagtttcagataaaaca

SEQ ID NO: 83 - DNA, 60 bp, synthetic construct
  source 1..60: mol_type = other DNA
agtgtcattttaagggacatttttatgacttttatgtgtatgtttatgtagaaatttgga

SEQ ID NO: 84 - DNA, 60 bp, synthetic construct
  source 1..60: mol_type = other DNA
actcacttcttttcaggtgtagctacaattgtgtaatgtacaatattagagaaaggacag

SEQ ID NO: 85 - DNA, 60 bp, synthetic construct
  source 1..60: mol_type = other DNA
cctgggagcaaatgaacaatagctaagtgtcttggtatttaaagagtaaattatttgtgg

SEQ ID NO: 86 - DNA, 60 bp, synthetic construct
  source 1..60: mol_type = other DNA
ccaagaatatatgctacagatataagacagacatggtttggtcctatatttctagtcatg

SEQ ID NO: 87 - DNA, 60 bp, synthetic construct
  source 1..60: mol_type = other DNA
atgctttcctaaatcagatgttttggtcaagtagtttgactcagtataggtagggagata

SEQ ID NO: 88 - DNA, 60 bp, synthetic construct
  source 1..60: mol_type = other DNA
atttgtgtggacaaaaatatttacacttagggtttggagctattcaagaggaaatgtcac

SEQ ID NO: 89 - DNA, 60 bp, synthetic construct
  source 1..60: mol_type = other DNA
aaatatactatgtttgcgaaccttggtagctatgatgagagctattatcatctgtggtgg

SEQ ID NO: 90 - DNA, 60 bp, synthetic construct
  source 1..60: mol_type = other DNA
tcaatgaaagttcaagaacctcctgtacttaaacacgattcgcaacgttctgttattttt

SEQ ID NO: 91 - DNA, 60 bp, synthetic construct
  source 1..60: mol_type = other DNA
accttgatagttcaccacgtctgatggatccctgttttaaataaaaacgattcactttaa

SEQ ID NO: 92 - DNA, 60 bp, synthetic construct
  source 1..60: mol_type = other DNA
taaaatacttcaatcctggattcacagtgggaacaagtttctattaaaaggcaaatgctg

SEQ ID NO: 93 - DNA, 60 bp, synthetic construct
  source 1..60: mol_type = other DNA
ggctgtgaacaatgttaaatagcatcagtttgtccaatagttttaaaggccataatcatc

SEQ ID NO: 94 - DNA, 60 bp, synthetic construct
  source 1..60: mol_type = other DNA
acgagtaccggcatgttatgttacccagagaactttccaaacaagtacctaaaactcatc

SEQ ID NO: 95 - DNA, 60 bp, synthetic construct
  source 1..60: mol_type = other DNA
attttttagaaaatacacacttttcaggagaaacctgagcatgattttggattctccacc

SEQ ID NO: 96 - DNA, 60 bp, synthetic construct
  source 1..60: mol_type = other DNA
cagctcagaccatttcctaatcagttgaaagggaaacaagtatttcagtctcaaaattga

SEQ ID NO: 97 - DNA, 60 bp, synthetic construct
  source 1..60: mol_type = other DNA
cactgcagactctcaagagatcaatcaaattgccagaaacagtttggtttttcatatgga

SEQ ID NO: 98 - DNA, 60 bp, synthetic construct
  source 1..60: mol_type = other DNA
tgaaactttcttctgatgagtttctttaacgtacaggatggagtaaaacaaatggtacag

SEQ ID NO: 99 - DNA, 60 bp, synthetic construct
  source 1..60: mol_type = other DNA
caattcttgagagttaatgtgatcatgatattgcaaacaactataaatggtctctaggcc

SEQ ID NO: 100 - DNA, 60 bp, synthetic construct
  source 1..60: mol_type = other DNA
gaaggaaacaccgagtctctgtataatctatttacataaaatgggtgatatgcgaacagc

SEQ ID NO: 101 - DNA, 60 bp, synthetic construct
  source 1..60: mol_type = other DNA
agcaacctgggccttgtactgtctgtgtttttaaaaccactaaagtgcaagaattacatt

SEQ ID NO: 102 - DNA, 60 bp, synthetic construct
  source 1..60: mol_type = other DNA
gggaatttgatgcagtaaagattaccctgttttatgattgttccttgaaagtcaaatggg

SEQ ID NO: 103 - DNA, 60 bp, synthetic construct
  source 1..60: mol_type = other DNA
taaggctaatgataccaatgagggttggtttattatcaaacctgaatagctgtggtttct

SEQ ID NO: 104 - DNA, 60 bp, synthetic construct
  source 1..60: mol_type = other DNA
tggggagatacatcttatagagttagaaatagaatctgaatttctaaagggagattctgg

SEQ ID NO: 105 - DNA, 60 bp, synthetic construct
  source 1..60: mol_type = other DNA
tatcttgaaactgaccaaacgcttattgtgtaagataaaccagttgaatcattgaggatc

SEQ ID NO: 106 - DNA, 60 bp, synthetic construct
  source 1..60: mol_type = other DNA
ttctctgaaggaatcatgttcagtgttcgaccacctaagaaaagttggaaaaagatcttc

SEQ ID NO: 107 - DNA, 60 bp, synthetic construct
  source 1..60: mol_type = other DNA
tgtcatagacatgtattggggagcttccaattagcatacatagacacatgtgtcagtggc

SEQ ID NO: 108 - DNA, 60 bp, synthetic construct
  source 1..60: mol_type = other DNA
tgtccatgctacaagaagttatgagccttgttctaagtacagatgaaccttgtatttgtg

SEQ ID NO: 109 - DNA, 60 bp, synthetic construct
  source 1..60: mol_type = other DNA
atcccgatttcagtcagacaaatactcatttcagagattctatacttcatggaatcaaga

SEQ ID NO: 110 - DNA, 60 bp, synthetic construct
  source 1..60: mol_type = other DNA
agttactttcttaatgtgacctagcaataggcatagctacgtggcactatattctggcca

SEQ ID NO: 111 - DNA, 60 bp, synthetic construct
  source 1..60: mol_type = other DNA
gaaatctctctacacagatgagtcatccaaacctgggaaaaaataaaagaactgcaatca

SEQ ID NO: 112 - DNA, 60 bp, synthetic construct
  source 1..60: mol_type = other DNA
aaattgctaagtggaatgcatgaattgcattatgttctctggtaacacgtagagttcaga

SEQ ID NO: 113 - DNA, 60 bp, synthetic construct
  source 1..60: mol_type = other DNA
ccaaaggtcttggtacaaccagctgcccattttgtgaaatttttatgtagaataaacatt

SEQ ID NO: 114 - DNA, 60 bp, synthetic construct
  source 1..60: mol_type = other DNA
gtttcgggtctttacctcatagtatgaaattagtaagacactgcatagattttgccctga

SEQ ID NO: 115 - DNA, 60 bp, synthetic construct
  source 1..60: mol_type = other DNA
gagtacggatgggaaactattgtgcacaagtctttccagaggagtttcttaatgagatat

SEQ ID NO: 116 - DNA, 60 bp, synthetic construct
  source 1..60: mol_type = other DNA
tattttatcagcactttatgcacgtattattgacattaatacctaatcggcgagtgccca

SEQ ID NO: 117 - DNA, 60 bp, synthetic construct
  source 1..60: mol_type = other DNA
tgccctatggaaaacttgtccaaataacatttcttgaacaataggagaacagctaaattg

SEQ ID NO: 118 - DNA, 60 bp, synthetic construct
  source 1..60: mol_type = other DNA
ctccttgtcattgaccttagctaaaccatggcaattcataaatagaggaaacattaatga

SEQ ID NO: 119 - DNA, 60 bp, synthetic construct
  source 1..60: mol_type = other DNA
ctgaacgagaacaagaatcagaagaagaaatgtgactttgatgagcttccagtttttcta

SEQ ID NO: 120 - DNA, 60 bp, synthetic construct
  source 1..60: mol_type = other DNA
tatattatcagtctgtaccagtagaccagtaccctaactactgaaaagaatatggcagtt

SEQ ID NO: 121 - DNA, 60 bp, synthetic construct
  source 1..60: mol_type = other DNA
agtaacgctaactttgtacggacgatgtctcatggattaaataatattctttatggcagt

SEQ ID NO: 122 - DNA, 60 bp, synthetic construct
  source 1..60: mol_type = other DNA
gtggatctacctcagttaaacagttgggtgctattactaagtctgtcaaattaaattgga

SEQ ID NO: 123 - DNA, 60 bp, synthetic construct
  source 1..60: mol_type = other DNA
cattctaaagggaaatcagtaaaatgtcttgataattggtatccaaatcacttgtgtgcc

SEQ ID NO: 124 - DNA, 60 bp, synthetic construct
  source 1..60: mol_type = other DNA
ccaaagacaaacgattagaagatggctatttcagaataggaaaatttgagaatggtgttg

SEQ ID NO: 125 - DNA, 60 bp, synthetic construct
  source 1..60: mol_type = other DNA
caaacttcctgacactacttccatatttgcactaaaggagattcagctacaaaaggaggc

SEQ ID NO: 126 - DNA, 60 bp, synthetic construct
  source 1..60: mol_type = other DNA
accttcctatgaagatcatggaatcaaatacgggacattgaactaatacttggactttga

SEQ ID NO: 127 - DNA, 60 bp, synthetic construct
  source 1..60: mol_type = other DNA
ggctaacacaatctaattttggtttaagagacaaatctagagtctcaaatgatctcagag

SEQ ID NO: 128 - DNA, 60 bp, synthetic construct
  source 1..60: mol_type = other DNA
tggacccttaaatatgactaaaatcacagcaatattgttacatacgggttatatgccaac

SEQ ID NO: 129 - DNA, 60 bp, synthetic construct
  source 1..60: mol_type = other DNA
taagcattgtgaaggaagattaatatagccaaataactagagtgatcagttctaccagag

SEQ ID NO: 130 - DNA, 60 bp, synthetic construct
  source 1..60: mol_type = other DNA
cctggataaaagtactgtatgattttgtgatggatgatacaataagtccctactcaagaa

SEQ ID NO: 131 - DNA, 60 bp, synthetic construct
  source 1..60: mol_type = other DNA
gctttgttactttgttaggtacgaatcacataagggagattgtatacaagttggagcaat

SEQ ID NO: 132 - DNA, 60 bp, synthetic construct
  source 1..60: mol_type = other DNA
taaaagatgaagaaagctattaggtatatttgtacatgactgcaaatgagtctatgcccg

SEQ ID NO: 133 - DNA, 60 bp, synthetic construct
  source 1..60: mol_type = other DNA
gtgtgttatctttatatgtcaaactggttgaacactgtaatgagaataaactgcacagag

SEQ ID NO: 134 - DNA, 60 bp, synthetic construct
  source 1..60: mol_type = other DNA
gattattgtacgaagtgtctctgtaattatcatactactaaagactgttcagatggcaag

SEQ ID NO: 135 - DNA, 60 bp, synthetic construct
  source 1..60: mol_type = other DNA
catttgtattaatggaatactaagtccctctgtgatttctgaaccaagctattcctaggc

SEQ ID NO: 136 - DNA, 60 bp, synthetic construct
  source 1..60: mol_type = other DNA
atgaagagatttctcaagctattcttgatttcagaaacgcaaaaaatgggtttgaaaggg

SEQ ID NO: 137 - DNA, 60 bp, synthetic construct
  source 1..60: mol_type = other DNA
agccaatcatgagtacgtaaagtgatttttgctctctgtgtacaacttttaaaatctgac

SEQ ID NO: 138 - DNA, 60 bp, synthetic construct
  source 1..60: mol_type = other DNA
atcctagaccatattttcaagtcatcttagcagctaggattctcaaatggaagtgttata

SEQ ID NO: 139 - DNA, 60 bp, synthetic construct
  source 1..60: mol_type = other DNA
agtgatttcatgctagaaaaattggaaactaaaagtgtgtagctaggttatttcggagtg

SEQ ID NO: 140 - DNA, 60 bp, synthetic construct
  source 1..60: mol_type = other DNA
gctaagccaagtagtagcagtaaaacttctgatcctctagcatcaaaaactacaactaca

SEQ ID NO: 141 - DNA, 60 bp, synthetic construct
  source 1..60: mol_type = other DNA
ggaaagaagttgaaagcatcttgaagaaaaactcagattggatatgggattggtcaagtc

SEQ ID NO: 142 - DNA, 60 bp, synthetic construct
  source 1..60: mol_type = other DNA
acctggatatgtctgtgaggctcctgaaaggagacaaataaagtcaatatatttgcacaa

SEQ ID NO: 143 - DNA, 60 bp, synthetic construct
  source 1..60: mol_type = other DNA
gggtatgaaagatgagtgtctgtaaaaatccttcttagaaatgtatttcctcaagactct

SEQ ID NO: 144 - DNA, 60 bp, synthetic construct
  source 1..60: mol_type = other DNA
cagatggcaagattgagtttatttcaacaatggaaggatataagtatccagtatatggtg

SEQ ID NO: 145 - DNA, 60 bp, synthetic construct
  source 1..60: mol_type = other DNA
gaaactgtgtcaccctaaagaagcatataatcatagcattaaaaatgcacacattactcc

SEQ ID NO: 146 - DNA, 60 bp, synthetic construct
  source 1..60: mol_type = other DNA
caagcgtgtttctagagaacagttgagagagaatctcaagattctacttggtggtttgct

SEQ ID NO: 147 - DNA, 60 bp, synthetic construct
  source 1..60: mol_type = other DNA
ccgacaagaggagatcattttagatattaccgaaatgaagaaagcttgcaattagtgaac

SEQ ID NO: 148 - DNA, 60 bp, synthetic construct
  source 1..60: mol_type = other DNA
taatagcaaaatttaacccgttactctttaaccttgtactggaaattctaagcagtgcag

SEQ ID NO: 149 - DNA, 60 bp, synthetic construct
  source 1..60: mol_type = other DNA
cttcctacctctggtgatggtttccacaggaacaacagcatctttcaccaagatgggtgg

SEQ ID NO: 150 - DNA, 60 bp, synthetic construct
  source 1..60: mol_type = other DNA
aaatcattcggtaaatccaaactgctatgcaaaagttatgatggttaacggtgatcacag

SEQ ID NO: 151 - DNA, 60 bp, synthetic construct
  source 1..60: mol_type = other DNA
ttgggtttctagtcctccttaccatcatctccatatgagagtgtgaaaataggaacacgt

SEQ ID NO: 152 - DNA, 60 bp, synthetic construct
  source 1..60: mol_type = other DNA
gctggtggagtagcagatgatattaatactaacaaaaaagaaggaatttcagatgttgtg

SEQ ID NO: 153 - DNA, 60 bp, synthetic construct
  source 1..60: mol_type = other DNA
tcacccagaaccaatgcggtgtttcttaatgtttgcacaaatttccttaaaaatcaactt

SEQ ID NO: 154 - DNA, 60 bp, synthetic construct
  source 1..60: mol_type = other DNA
caggacttctctttagtcagggcatgctttattagtgaggagaaaacaattccttagaag

SEQ ID NO: 155 - DNA, 60 bp, synthetic construct
  source 1..60: mol_type = other DNA
ccctgtgctatcgtaagtttgttttgagcactgcattcactttaaaattctggaggaaca

SEQ ID NO: 156 - DNA, 60 bp, synthetic construct
  source 1..60: mol_type = other DNA
caacatatttcagttggaaaatttgtatgcagtaatcagccaatgtatttatcggcatcg

SEQ ID NO: 157 - DNA, 60 bp, synthetic construct
  source 1..60: mol_type = other DNA
cccccattctggaaggttttgttatcttcggaagaaccccaattatgatctctaagtgac

SEQ ID NO: 158 - DNA, 60 bp, synthetic construct
  source 1..60: mol_type = other DNA
tgtccccagggatcaaacagaagcagccgtgggcaaaatacaatttcatttaacaaattg

SEQ ID NO: 159 - DNA, 60 bp, synthetic construct
  source 1..60: mol_type = other DNA
aaacagcattatggagttaaaagatttttacaactgggtcttgattttgatgtgagctgg

SEQ ID NO: 160 - DNA, 60 bp, synthetic construct
  source 1..60: mol_type = other DNA
tccagacgcactgatctttgcaaaggagacttaatttcaaatctgtaattaccatacata

SEQ ID NO: 161 - DNA, 60 bp, synthetic construct
  source 1..60: mol_type = other DNA
catttggctgtcagaaattataccgagtctactgggtataacatgtctcacttggaaagc

SEQ ID NO: 162 - DNA, 60 bp, synthetic construct
  source 1..60: mol_type = other DNA
ttaaaggcaaaactgtgctctttattttaaaaaacactgataatcacactgcggtaggtc

SEQ ID NO: 163 - DNA, 60 bp, synthetic construct
  source 1..60: mol_type = other DNA
aaggtgctgtcatatatcttggaatgaatgacctaaaatcattttaaccattgctactgg

SEQ ID NO: 164 - DNA, 60 bp, synthetic construct
  source 1..60: mol_type = other DNA
ggatgtaaatcctgagctcaaatctctgttactccattactgtgatttctggctgggtca

SEQ ID NO: 165 - DNA, 60 bp, synthetic construct
  source 1..60: mol_type = other DNA
cctcaagagtatgtataatttgaagagatactttgtaactatgcttgggtgatattgagc

SEQ ID NO: 166 - DNA, 60 bp, synthetic construct
  source 1..60: mol_type = other DNA
ttcacagaatagcacaaactacaattaaaactaagcacaaagccattctaagtcattggg

SEQ ID NO: 167 - DNA, 60 bp, synthetic construct
  source 1..60: mol_type = other DNA
ccagcacataggagagatgagcttcctacagcacaacaaatgtgaatgcagaccaaagaa

SEQ ID NO: 168 - DNA, 60 bp, synthetic construct
  source 1..60: mol_type = other DNA
gagaaacattgtatattttgcaaaaacaagatgtttgtagctgtttcagagagagtacgg

SEQ ID NO: 169 - DNA, 60 bp, synthetic construct
  source 1..60: mol_type = other DNA
tactttttggaaaagaataaaccaagaattgattgggcacatcatttcaagaagtccctc

SEQ ID NO: 170 - DNA, 60 bp, synthetic construct
  source 1..60: mol_type = other DNA
atggagttgctagtaaagcgaagctgattatcctggaaaacactatttacctattttcca

SEQ ID NO: 171 - DNA, 60 bp, synthetic construct
  source 1..60: mol_type = other DNA
gactgctagtggataataacatcttgactacttaaaaaagggacatattgaaaatcctgg

SEQ ID NO: 172 - DNA, 60 bp, synthetic construct
  source 1..60: mol_type = other DNA
catgttacctggactggaacagactgtgaatatagcagaaggttccaagaactctggtgt

SEQ ID NO: 173 - DNA, 60 bp, synthetic construct
  source 1..60: mol_type = other DNA
gagaccaggtgcttcaaaacttaggctcggtagaatcttactcagaagaaaaagcaaaaa

SEQ ID NO: 174 - DNA, 60 bp, synthetic construct
  source 1..60: mol_type = other DNA
ggattcaacccaaatgatttctcatcaggtgattcttggttgtagcaaagttcatgtgaa

SEQ ID NO: 175 - DNA, 60 bp, synthetic construct
  source 1..60: mol_type = other DNA
agaactcttgattttgtacatagtcctctggtctatctcatgaaacctcttctcagacca

SEQ ID NO: 176 - DNA, 60 bp, synthetic construct
  source 1..60: mol_type = other DNA
aattggtaaacatcatgttcctgatgataacccagtagcaaaaacatttgtactgagtgg

SEQ ID NO: 177 - DNA, 60 bp, synthetic construct
  source 1..60: mol_type = other DNA
catcagtcttgggaaatttgaactttgatcaacttaactaaagaaggaagggtagtaaga

SEQ ID NO: 178 - DNA, 60 bp, synthetic construct
  source 1..60: mol_type = other DNA
ttagggccctacgtaataggctaattgtactgctcttagaatgtaagcgttcacgaaaat

SEQ ID NO: 179 - DNA, 60 bp, synthetic construct
  source 1..60: mol_type = other DNA
gagtctttgggataccattaaaaagaagaaaatttcagcctctacaagtcacaacagaag

SEQ ID NO: 180 - DNA, 60 bp, synthetic construct
  source 1..60: mol_type = other DNA
agagtgtgaaaaataggaacacgtgctctacctccatttagggatttgcttgggatacag

SEQ ID NO: 181 - DNA, 60 bp, synthetic construct
  source 1..60: mol_type = other DNA
aactttttagggcaaagttaacactgaaagttctagcttaagtgttgaaacttttgtggg

SEQ ID NO: 182 - DNA, 60 bp, synthetic construct
  source 1..60: mol_type = other DNA
acttagcattttctgcatctccacttggcattagctaaaaccttccatgtcaagattcag

SEQ ID NO: 183 - DNA, 60 bp, synthetic construct
  source 1..60: mol_type = other DNA
ttttgatgagaatgaatcttggtacttagatgacaacatcaaaacatactctgatcaccc

SEQ ID NO: 184 - DNA, 60 bp, synthetic construct
  source 1..60: mol_type = other DNA
ttcccttcaatactcctaaaaccaaagaaggatattactaccgtcaagtctttgaacgcc

SEQ ID NO: 185 - DNA, 60 bp, synthetic construct
  source 1..60: mol_type = other DNA
tcctgtcctgctcattatgccacttccttttaactgccaagaaattttttaaaataaata

SEQ ID NO: 186 - DNA, 60 bp, synthetic construct
  source 1..60: mol_type = other DNA
caaaaactcagatctatcttaagagtgaccaggaagaggttcattgaaataatcatgcat

SEQ ID NO: 187 - DNA, 60 bp, synthetic construct
  source 1..60: mol_type = other DNA
catacggttttgtttggaggatggcttctgctgctaaaaatacaaaagtttggaaaccgc

SEQ ID NO: 188 - DNA, 60 bp, synthetic construct
  source 1..60: mol_type = other DNA
cagagggaccttatttaaacataagtgctgtgacttcggtgaattttcaatttaaggtat

SEQ ID NO: 189 - DNA, 60 bp, synthetic construct
  source 1..60: mol_type = other DNA
gtttgtgaaactgttaaggtcctttctaaattcctccattgtgagataaggacagtgtca

SEQ ID NO: 190 - DNA, 60 bp, synthetic construct
  source 1..60: mol_type = other DNA
ttaaccagctgtaaaacacagacctttatcaagagtaggcaaagattttcaggattcata

SEQ ID NO: 191 - DNA, 60 bp, synthetic construct
  source 1..60: mol_type = other DNA
ggggatgaatagaaaacctgtaagctttgatgttctggttacttctagtaaattcctgtc

SEQ ID NO: 192 - DNA, 60 bp, synthetic construct
  source 1..60: mol_type = other DNA
gtgataaagtacctgatccaaatgttatgagaatactggacgagaattgaacgaaattga

SEQ ID NO: 193 - DNA, 60 bp, synthetic construct
  source 1..60: mol_type = other DNA
tgcagcagtactactgtcaacatagtgtaaatggttctcaaaagcttaccagtgtggact

SEQ ID NO: 194 - DNA, 60 bp, synthetic construct
  source 1..60: mol_type = other DNA
gcatgagtcacaattacaaagttttgagcggttttgtaatttgacatttaggaaagtctc

SEQ ID NO: 195 - DNA, 60 bp, synthetic construct
  source 1..60: mol_type = other DNA
ttattcgaagacacagaagttgggcaagtcaaatgttgtgtcgtcagttgtgcatccgtt

SEQ ID NO: 196 - DNA, 60 bp, synthetic construct
  source 1..60: mol_type = other DNA
tgtactggcaggctcgttttacctgattctagaatatttaagaatctaaaaataaagggc

SEQ ID NO: 197 - DNA, 60 bp, synthetic construct
  source 1..60: mol_type = other DNA
gtggcctataacttacttgtcaacaactgtgaacattttgtgacattgcttcgctatgga

SEQ ID NO: 198 - DNA, 60 bp, synthetic construct
  source 1..60: mol_type = other DNA
ccaggacgccactcatttcatctcatttaagggaaaaatatatatctatctatttgagga

SEQ ID NO: 199 - DNA, 60 bp, synthetic construct
  source 1..60: mol_type = other DNA
cggagcgcagggtacttggcgtataataagccatcaataatttatgggtgaaattgagag

SEQ ID NO: 200 - DNA, 60 bp, synthetic construct
  source 1..60: mol_type = other DNA
cagagctacaactaggaaaattagagtggtagtagtcacttatttaagaattcattcagg

SEQ ID NO: 201 - DNA, 60 bp, synthetic construct
  source 1..60: mol_type = other DNA
ttggtagttaaccctaactacttgctcgaagattgagatagtgaaagtaactgaccagag

SEQ ID NO: 202 - DNA, 60 bp, synthetic construct
  source 1..60: mol_type = other DNA
gcgtgagcatgtcagtattctagtccagtatttgccagtttccaagtaaaagcttttgtg

SEQ ID NO: 203 - DNA, 60 bp, synthetic construct
  source 1..60: mol_type = other DNA
gctgtgccattcaatgtttgatgcataattggaccttgaatcgataagtgtaaatacagc

SEQ ID NO: 204 - DNA, 60 bp, synthetic construct
  source 1..60: mol_type = other DNA
ccaagaaggaaaatgtcaaaattagtgatgagggaatagcttatcttgttaaagtgtcag

SEQ ID NO: 205 - DNA, 60 bp, synthetic construct
  source 1..60: mol_type = other DNA
tgctttaagtgaatggcagtcccttgtcttattcagaatataaaattcagtctgaatggc

SEQ ID NO: 206 - DNA, 60 bp, synthetic construct
  source 1..60: mol_type = other DNA
aggttggcagtaaggcagggtcccatttctcactgagaagattgtgaatatttccatatg

SEQ ID NO: 207 - DNA, 60 bp, synthetic construct
  source 1..60: mol_type = other DNA
gtgcaaatagaattagcagtaagaagctactctagctaatttgccatttcacttaaatgg

SEQ ID NO: 208 - DNA, 60 bp, synthetic construct
  source 1..60: mol_type = other DNA
gatacagcctacataaagactgttatgatcgctttgattttaaagttcattggaactacc

SEQ ID NO: 209 - DNA, 60 bp, synthetic construct
  source 1..60: mol_type = other DNA
caacatttacattgtaattcaatagacgctactactacaaaggagctttattcttccagc

SEQ ID NO: 210 - DNA, 60 bp, synthetic construct
  source 1..60: mol_type = other DNA
caacagtattgcgttgtcagactaggaaagctaaacgaacaaaatggttttagttttgct

SEQ ID NO: 211 - DNA, 60 bp, synthetic construct
  source 1..60: mol_type = other DNA
ctggttgtccaactaccatatgaagctagaaaatgcacaaacgatattccttatctgtaa

SEQ ID NO: 212 - DNA, 60 bp, synthetic construct
  source 1..60: mol_type = other DNA
ggcatcagggatcacatcactcttaacggctgttacttaaacaactattttttggtttgg

SEQ ID NO: 213 - DNA, 60 bp, synthetic construct
  source 1..60: mol_type = other DNA
tgaaaatgtatttgtagtcacggactttcaggattctgtctttaatgacctctacaaggc

SEQ ID NO: 214 - DNA, 60 bp, synthetic construct
  source 1..60: mol_type = other DNA
agaccaggtctctattttgaggaagaaataccgagacattgagcgactttgaggaatccg

SEQ ID NO: 215 - DNA, 60 bp, synthetic construct
  source 1..60: mol_type = other DNA
aagtcatgacacagtattcgctctttttctgaatgtttacatagagattcatcactgcag

SEQ ID NO: 216 - DNA, 60 bp, synthetic construct
  source 1..60: mol_type = other DNA
cagtaagtacgggaaaaaatgtttactaacttcctcagagattcgtgatacgcgtttctc

SEQ ID NO: 217 - DNA, 60 bp, synthetic construct
  source 1..60: mol_type = other DNA
ctttgaatggacataaaaattctgcttgttaagaacaagttgagctctggtaactgatct

SEQ ID NO: 218 - DNA, 60 bp, synthetic construct
  source 1..60: mol_type = other DNA
tgactgatgtgtctgaaaatgctaaggatcttattcgaaggctcatttgtagcagagaac

SEQ ID NO: 219 - DNA, 60 bp, synthetic construct
  source 1..60: mol_type = other DNA
ctctgaaagaagaagttcaaaagctggatgatctttaccaacaaaaaattaaggaagcag

SEQ ID NO: 220 - DNA, 60 bp, synthetic construct
  source 1..60: mol_type = other DNA
atctgtggttattcgaacctttattactagtgacttcatgactggtatacctgcaacacc

SEQ ID NO: 221 - DNA, 60 bp, synthetic construct
  source 1..60: mol_type = other DNA
tccaccccaggacgccactcatttcatctcatttaagggaaaaatatatatctatctatt

SEQ ID NO: 222 - DNA, 60 bp, synthetic construct
  source 1..60: mol_type = other DNA
ggccctctcttctcacctttgttttttgttggagtgtttctaataaacttggattctcta

SEQ ID NO: 223 - DNA, 60 bp, synthetic construct
  source 1..60: mol_type = other DNA
ctgggttgatacctgaaagaatcctgtcttatttggtctccataatcctttgaatggaaa

SEQ ID NO: 224 - DNA, 60 bp, synthetic construct
  source 1..60: mol_type = other DNA
agtaccctgatatactgaattttgtggatgatttggaacctttagacaaagctagtaaag

SEQ ID NO: 225 - DNA, 60 bp, synthetic construct
  source 1..60: mol_type = other DNA
aagactttcttactgacctgaataacttcagaaccacattcatgcaagcaataaaggaga

SEQ ID NO: 226 - DNA, 60 bp, synthetic construct
  source 1..60: mol_type = other DNA
tttagtggtccgttgcctctgaagatgtaaacaaacaaatacactatttctgggaacatt

SEQ ID NO: 227 - DNA, 60 bp, synthetic construct
  source 1..60: mol_type = other DNA
atagaatatgtatatgtattctttgtctaccaactaccaaagaaacaaatactcctcagt

SEQ ID NO: 228 - DNA, 60 bp, synthetic construct
  source 1..60: mol_type = other DNA
acattgcttacttaaaagctacatagccctatcgaaatgcgaggattaatgctttaatgc

SEQ ID NO: 229 - DNA, 60 bp, synthetic construct
  source 1..60: mol_type = other DNA
accataaggcaattgagcacataacgaaaaatgatgcaataagaatgtatgcactctctt

SEQ ID NO: 230 - DNA, 60 bp, synthetic construct
  source 1..60: mol_type = other DNA
cagcctttcctcatgtcaacacagttcacaatatagttttcaaagtacagtttaaaactc

SEQ ID NO: 231 - DNA, 60 bp, synthetic construct
  source 1..60: mol_type = other DNA
cctccccaaaataattagtaactggttgttctacttggtaatttgacaccctgttaataa

(19) United States
(12) Patent Application Publication
(10) Pub. No.: US 2024/0060138 A1
(43) Pub. Date: Feb. 22, 2024

(54) BREAST CANCER-RESPONSE PREDICTION SUBTYPES

(71) Applicant: The Regents of The University of California, Oakland, CA (US)

(72) Inventors: Laura J. van't Veer, San Francisco, CA (US); Denise Wolf, San Francisco, CA (US); Christina Yau, San Francisco, CA (US); Laura Esserman, San Francisco, CA (US)

(21) Appl. No.: 18/174,491

(22) Filed: Feb. 24, 2023

Related U.S. Application Data
(60) Provisional application No. 63/314,065, filed on Feb. 25, 2022.
(60) Provisional application No. 63/341,579, filed on May 13, 2022.

(51) Int. Cl. C12Q 1/6886

(52) U.S. Cl. CPC C12Q 1/6886; C12Q 2600/106; C12Q 2600/158

(57) ABSTRACT

The disclosure describes a tumor subtyping schema for selection of therapies to treat Stage II and Stage III breast cancers.

### CROSS-REFERENCE TO RELATED APPLICATIONS

This application claims benefit of priority to U.S. Provisional Application No. 63/341,579, filed May 13, 2022 and U.S. Provisional Application No. 63/314,065 filed Feb. 25, 2022, each of which is incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under grant no. P01 CA210961 awarded by The National Institutes of Health. The government has certain rights in the invention.

### REFERENCE TO A SEQUENCE LISTING

The contents of the electronic sequence listing (081906-1375781-245120US_SLxml; Size: 213,614 bytes; and Date of Creation: Jul. 28, 2023) is herein incorporated by reference in its entirety.

### BACKGROUND OF THE INVENTION

Though breast cancer treatment has improved over the past decades, over 40,000 women die annually in the US alone and worldwide, on average one in three patients will die of their disease (DeSantis et al., 2015). Patients who achieve pathologic complete response (pCR) after neoadjuvant therapy, defined by the absence of invasive disease in breast and lymph nodes, have excellent long-term outcomes (Spring et al., 2020; Yee et al., 2020). By improving pCR rates in the early disease setting, we can reduce the risk of subsequent metastatic disease and death from breast cancer. The I-SPY2 trial is an ongoing multicenter, Phase II neoadjuvant platform trial for high-risk, early-stage breast cancer designed to rapidly identify new treatments and treatment combinations with increased efficacy compared to standard-of-care (sequential weekly paclitaxel followed by doxorubicin/cyclophosphamide (T-AC) chemotherapy). In I-SPY2, multiple novel treatment regimens are simultaneously and adaptively randomized against the shared control arm (Chien et al., 2019; Nanda et al., 2020; Park et al., 2016; Rugo et al., 2016). The primary efficacy endpoint is pCR (Yee et al., 2020).

The goal of the trial is to assess the activity of new drugs, typically combined with weekly paclitaxel, in a priori defined biomarker subsets based on hormone receptor (HR), Human Epidermal Growth Factor Receptor-2 (HER2) expression, and MammaPrint (MP) status. Among HR+HER2-patients, only MammaPrint (MP) high cases are eligible for the trial. For all patients, tumor biology is further subdivided into high (MPT) or ultra-high (MP2) status (Chien et al., 2019; Nanda et al., 2020; Park et al., 2016; Rugo et al., 2016). An experimental arm “graduates” when it reaches ≥85% predictive probability of demonstrating superiority to control in a future 1:1 randomized 300-patient Phase III neoadjuvant trial in the most responsive subset (Chien et al., 2019; Clark et al., 2021; Nanda et al., 2020; Park et al., 2016; Rugo et al., 2016).

It is well established that HR/HER2 subtyping is well suited for predicting response to endocrine and HER2-targeted agents (Waks and Winer, 2019). However, the landscape of targeted breast cancer therapeutics is expanding. Breast cancer treatment now includes platinum agents, PARP inhibitors, PIK3CA inhibitors, mTOR inhibitors, dual HER2-targeting regimens, and immunotherapy for specific HR/HER2-defined subtypes (Bergin and Loi, 2019; McAndrew and Finn, 2020; Wuerstlein and Harbeck, 2017). The aggregate mechanisms of action of the compendium of currently clinically available targeted therapeutics for breast cancer extends well beyond the biology that HER and HR expression captures.

Within the I-SPY2 biomarker program, there are two primary biomarker platforms assayed at the pretreatment time-point—gene expression arrays and reverse phase protein arrays (RPPA). In the case of RPPA, upfront enrichment and purification of tumor epithelium, stromal, and intra-tumoral immune cell compartments via laser capture microdissection (LCM) is performed prior to separately assaying each population. Biomarkers are classified as standard, qualifying, or exploratory. Standard biomarkers are routinely used, US Food and Drug Administration cleared or approved, or have investigational device exemption (IDE) status (i.e. HR, HER2, MammaPrint, MRI functional tumor volume) and employed for clinical decision making. Qualifying biomarkers are pre-specified for analysis based on existing evidence suggesting a role in treatment response prediction and are tested in a CLIA setting; they may vary from drug to drug and are tested prospectively for their specific response-predictive value using a pre-specified statistical framework (Wolf et al., 2017, 2020a; Wulfkuhle et al., 2018). Exploratory biomarkers are hypothesis-generating and include discovery efforts using clinical data to identify predictive biomarkers (Sayaman et al., 2020).

The goal of the trial is to assess the activity of various drugs in combination, mostly in combination with weekly paclitaxel, in various a priori defined biomarker subsets based on hormone receptor (HR) and Human Epidermal Growth Factor Receptor-2 (HER2) expression, and MammaPrint status. Among HR+HER2-patients, only MammaPrint (MP) high cases are eligible. For all patients, tumor biology is further subdivided into high (MP1) or ultra-high (MP2) status (Chien et al., 2020; Nanda et al., 2020; Park et al., 2016; Rugo et al., 2016; Pusztai et al., 2021). An experimental arm “graduates” when it reaches a≥85% predictive probability of demonstrating superiority to control in a future 1:1 randomized 300-patient phase 3 neoadjuvant trial in the most responsive subset (Chien et al., 2020; Nanda et al., 2020; Park et al., 2016; Rugo et al., 2016).

### BRIEF SUMMARY

The I-SPY2 trial and associated datasets provides an opportunity to develop new breast cancer subtype classifications because of its comprehensive multi-omic molecular characterization of all tumors and the diverse array of drugs targeting different molecular pathways. As of September Jan. 27, 2022, 2096 patients were randomized to I-SPY2, and 20 novel drugs were tested in the trial, of which 16 have completed evaluation. Experimental treatments include pan-HER2 inhibitors and anti-HER2 agents, PARP inhibitor/DNA damaging agent combinations, an AKT inhibitor, immunotherapy, and ANG1/2, IGF1R and HSP90 inhibitors added to standard of care chemotherapy. This disclosure is based, at least in part, on analyses across 10 arms of I-SPY2: the first 9 experimental arms that completed evaluation and the control arm. We determined that molecular subtyping categories incorporating biology outside of HR and HER2 status could be created to better inform treatment selection for individual patients and maximize efficacy (i.e., pCR rate) over the entire population.

As described herein, we summarized and further explored qualifying biomarker results across 10 arms of I-SPY2, combining information from standard and qualifying biomarkers to create biological treatment response-predicting subtypes (RPS) that represent better matches for the tested drugs than the standard HR/HER2-based subtypes (i.e., maximize pCR rate for a given drug, or class of agent, in a given subtype). Accordingly, the present disclosure provides a new RPS classification schema.

In one aspect, the disclosure provides a classification scheme to assign a Stage II or Stage III breast cancer patient to a treatment for which the patient has an increased likelihood of having a positive response. Described herein is a method of selecting a therapeutic treatment for a high-risk HER2+ or HER2-Stage II or Stage III breast cancer that is hormone receptor+ or hormone receptor−, the method comprising:
classifying the Stage II or Stage III breast cancer as having a positive or negative immune response profile for responding to an immunotherapy treatment, wherein a positive immune response profile is assigned by determining that the expression pattern of at least one panel of immune status genes reaches or exceeds a threshold that is associated with a high pathology complete response (pCR) rate for patients treated with an immune pathway-targeted therapy compared to patients treated with therapies that do not target the immune response; and a negative immune response profile is assigned by determining that the expression pattern is lower than the threshold;
classifying the Stage II or Stage III breast cancer as having a positive or negative DNA Repair Defect (DRD) profile for responding to a DNA repair treatment, wherein a positive DRD response profile is assigned by determining that the expression pattern of at least one panel of DRD status reaches or exceeds a threshold that is associated with a high pathology complete response (pCR) rate for patients treated with a DNA repair-targeted therapy compared to patients treated with therapies that do not target DNA repair; and a negative DRD response profile is assigned by determining that the expression pattern is lower than the threshold; and
assigning the breast cancer to a treatment subtype selected from the group consisting of HER2−/Immune-/DRD−, HER2−/Immune-/DRD+, HER2−/Immune+, HER2+/BP-HER2-type or Basal-type, and HER2+/BP-Luminal.-type.

In some embodiments, classifying the Stage II or Stage III breast cancer as having a positive or negative immune response profile comprises evaluating expression levels of at least one panel of immune status genes, and wherein the panel is selected from a TcellBcell biomarker panel, a dendritic biomarker panel, a chemokine biomarker panel, a MastCell biomarker panel, a STAT1 biomarker panel, and a B-cell biomarker panel as set forth in Table B.

In some embodiments, the breast cancer is hormone receptor-positive (HR+). I some emboidments, the breast cancer is HR+ and HER2−. In some embodiments, classifying the Stage II or Stage III breast cancer as having a positive or negative immune response profile comprises evaluating expression levels of B-cell and Mast-cell biomarker panels.

In some embodiments, the breast cancer is estrogen receptor-negative, progesterone receptor-negative and HER2-negative (triple negative). In some embodiments, classifying the Stage II or Stage III breast cancer as having a positive or negative immune response profile comprises evaluating expression levels of a dendritic cell panel and a STAT1 and/or chemokine panel. In some emobdiments, classifying the breast cancer as having a positive DRD profile comprises determining that the expression pattern of a VCpred_TN gene panel set forth in Table B falls within a range that is associated with a high pCR rate for patients treated with a therapeutic agent that targets DNA repair compared to patients treated with a therapy that does not target DNA repair.

In some embodiments, classifying the Stage II or Stage III breast cancer as having a positive DRD response profile comprises evaluating expression levels of a PARPi7 or PARPi7_plus_MP2 panel.

In some embodiments, Stage II breast cancer is classified as a high-risk HER2+ breast cancer by MammaPrint® analysis.

In some embodiments, the method of selecting a therapeutic treatment further comprises
selecting a DNA repair targeted therapy for a patient having a breast cancer assigned to the HER2−/Immune//DRD+ subtype, selecting an immune response therapy for a patient having a breast cancer assigned to the HER2−/Immune+ subtype; selecting a dual-anti-HER2 therapy for a patient assigned to the HER2+ that are not luminal subtype; selecting a combination therapy that comprises an AKT pathway-inhbitor for a patient assigned to the HER2+/BP-Luminal subtypes; and selecting neoadjuvant endocrine therapy for a patient assigned to the HER2−/Immune-/DRD-subtype. In illustrative embodiments, the immune response therapy is an PDL1/PD1 checkpoint inhibitor therapy, the DNA repair therapy is a platinum based therapy or PARP inhibitor; and the AKT pathway inhibitor is an AKT inhibitor.

In a further aspect, one of the biologies, e.g., DNA repair or immune response, can be represented by an additional or alternative gene profile representing the same biology.

### BRIEF DESCRIPTION OF THE DRAWINGS

FIG. 1A-1D. Trial design and data. FIG. 1A I-SPY2 trial schematic, FIG. 1B timeline of I-SPY2 investigational agents/combinations for the first 10 arms, FIG. 1C pCR rate across arms by receptor subtype (blue arrows=graduated; grey arrows=graduated in group containing subtype (e.g. HER2+ for HR+HER2+), FIG. 1D ISPY2-990 mRNA/RPPA Data Resource consort/schematic.

FIG. 2. Clustered heatmap of mechanism-of-action ‘qualifying’ biomarkers across 10 arms. Heatmap showing unsupervised clustering of mechanism-of-action biomarkers (rows) and patient samples (columns), with biomarkers annotated by platform (dark=mRNA) and pathway, and samples annotated by HR/HER2 status (dark=positive), MP1/2 class (dark=MP2), response (dark=pCR), receptor subtype, PAM50 subtype, TNBC subtype (7- and 4-classes), and arm. Clustering uses Pearson correlation and complete linkage, with clusters C1-7 defined by a dendrogram cutpoint of 1.5

FIG. 3. pCR association analysis of continuous mechanism-of-action biomarkers across 10 arms. This figure (sheet 6/33 and continuation (sheet 7/33) shows the pCR-association dot-plot showing the level and direction of association between each signature (column) and pCR in the population/arm as labeled (rows): Overall population, in all 10 arms, in a model adjusting for HR, HER2, and Tx (top row) and by arm, in a model adjusting for HR and HER2 (next 10 rows); HR+HER2− subset, in a model adjusting for arm (row 12) and within each of the 8 arms where HER2-negative patients were eligible (rows 13-20). Similarly, the remaining rows show pCR association results for TN (rows 21-29), HR+HER2+(rows 30-36) and HR-HER2+(rows 37-42) subsets, overall in a model adjusting for treatment arm and within each treatment arm. Key=red/blue dot indicates higher/lower levels ˜pCR; darker/lighter color intensity ˜higher/lower magnitude of coefficient of association (|exp(OR per unit standard deviation)\); size of dot ˜strength of association (1/p), with white background indicating p<0.05; X denotes missing data. For analysis in the overall population (rows 1-11), logistic regression models pCR ˜Biom+HR+HER2+Tx (all arms; row 1) and pCR-Biom+HR+HER2 (one arm; rows 2-11) were used; whereas within HR/HER2 subsets (rows 12-43), models pCR-Biom+Tx (all arms; rows 12, 21, 30 and 37) and pCR-Biom (one arm; rows 13-20, 22-29, 31-36, and 38-42) were used. Biomarkers (columns) are color annotated at the top for platform (dark=mRNA; light=RPPA) and pathway (see legend).

FIG. 4A-FIG. 4F Clinically motivated response-based biomarker-subsets. FIGS. 4A and 4B One-phenotype stratification: Pie charts showing prevalence of TN/Immune+(FIG. 4A, left) and TN/DRD+(FIG. 4B, left) subsets, respectively. pCR rates by biomarker subset in the VC and Pembro arms are shown in barplots (FIGS. 4A, 4B right). p-values shown are from Fisher's exact test (pCR˜biom). FIG. 4C Two-phenotype stratification: Sankey plot showing prevalence of Immune/DRD biomarker subsets in TNBC, with pCR rates in VC, Pembro and control shown in barplots to the right. FIG. 4D Immune-DRD stratification in HR+HER2−: Sankey showing prevalence of biomarker groups. FIG. 4E HER2+ stratification by BluePrint subtype. Prevalence of HER2+/BP_Luminal and HER2+/BP_Her2_or_Basal (Sankey diagram, left); and pCR rates in Ctr, TDM1/P and MK2206 arms (right). FIG. 4F Sankey diagram showing the collapse of Immune/DRD subtypes from 8 to 3 classes. In (FIG. 4C), # denotes patient subset too small to be evaluable (<5).

FIG. 5A-FIG. 5C. Integrated treatment response-predictive subtyping 5 (RPS-5) schema combining Immune, DRD, HER2, and BP_subtype phenotypes. FIG. 5A (sheet 13/33 and continuation sheets 14/33 and 15/33) Sankey diagram illustrates the relationship between receptor subtype and RPS-5 subtypes, with subtype prevalence and barplots on either side showing pCR rates by arm in each biomarker-defined subset*(highest in blue).

FIG. 5B In silico ‘thought experiment’ barplot showing pCR rates achieved in I-SPY2's control arm (black bar), experimental arms (orange bar); and estimated pCR rates if treatments had been ‘optimally’ assigned using receptor subtype (red bar; upper right text) or RPS-5 subtyping (blue bar, lower right text). Bar grouping to the left is for the overall population, and groupings to the right show pCR gains by HR/HER2 status. FIG. 5C Hazard-ratio (HR) for Distant Recurrence-Free Survival (DRFS) for pCR versus non-pCR by RPS-5 subtype. *pCR rates by receptor subtype (FIG. 5A) are calculated across the 987 patients of this biomarker analysis and may differ from the reported pCR in FIG. 1C which represents the Bayesian-estimated trial results of investigational arms versus appropriate controls. In (FIG. 5A), # denotes patient subset too small to be evaluable (<5), * denotes subtype not eligible for the arm, and p-values are from Fisher's exact test.

FIG. 6. Response-predictive subtyping schema characteristics diagram for 11+ example schemas. Compound diagram showing the characteristics of each breast cancer subtyping schema (columns), including the number and prevalence of classes (pie charts: 3-8 classes), constituent biomarkers (grid in purples (=present) and white (=absent) above pie charts), treatment arms with the highest pCR rate in one or more class (grid with turquoise (=selected) and cream (=not selected) squares labeled ‘Selected arms’), and in silico experiment stacked barplot showing pCR rates achieved in the control arm (black), experimental arms (orange); and estimated pCR rates if treatments had been optimally assigned using receptor subtype (red) or by the response-predictive schema in the column (blue and % pCR label). Top (pink bars) shows just the gain in pCR relative to receptor subtype.

FIG. 7A-FIG. 7B. Impact of subtyping schema on minimum required efficacy of new agent. FIG. 7A Sankey plot showing a variety of ways to combine Her2 low status with other phenotypes/biomarkers including Luminal vs. Basal and Immune/DRD. FIG. 7B scatter plot showing prevalence of HER2 low subset (x-axis) vs. the minimum pCR rate a HER2 low-targeting agent would have to achieve to equal that of the I-SPY2 agent with the highest response in that subset (minimum efficacy; y-axis).

FIG. 8A-FIG. 8D. Number of genes, phospho-proteins, and ‘qualifying’ biomarkers/signatures associated with pCR by arm. FIG. 8A Bar chart showing % arm-subtype pairs where a biomarker associates for pCR (y-axis) for each biomarker (x-axis), FIG. 8B pCR-association dot-plot for HER2+ subset showing the level and direction of association between each signature (column) and pCR in the population/arm as labeled (rows): all HER2+ in a model adjusting for Tx (top row) and by arm where HER2+ patients were eligible. Key=red/blue dot indicates higher/lower levels ˜pCR; size of dot ˜strength of association (1/p), with white background indicating p<0.05; X denotes missing data. FIGS. 8C, FIG. 8D % biomarker-receptor subtype pairs associated with pCR by arm, for the 27 qualifying biomarkers FIG. 8C and over the transcriptome as a whole FIG. 8D.

FIG. 9A-FIG. 9G. FIG. 9A Clustered heatmap of selected dichotomized (or binary/categorical) biomarkers (rows) and patient samples (columns), with samples annotated by receptor subtype, PAM50 subtype, TNBC subtypes (7- and 4-class), pCR, and arm. FIG. 9B Schematic showing how key biological phenotypes/biomarkers (third row) are combined to create I-SPY 2 subtypes (top row), standard receptor subtype (second row), and composite subsets (third row) that are then combined to create the ‘final’ integrated response subtyping schemas (fourth row). Broken lines/arrows indicate inclusion of a 3-state Her2 (HER2=0/low/+). Red arrows indicate biomarkers/phenotypes incorporated in resulting integrated response-predictive schemas. FIG. 9C boxplots showing the Vcpred_TN signature in pCR and non-pCR patients in the BrighTNess trial (NCT02032277; (Filho et al., 2021; Loibl et al., 2018)) in all carbo-containing arms (top) and by arm (bottom). FIG. 9D Sankey showing prevalence of HR+HER2− patients positive for Immune and/or DRD biomarkers, and barplots to the right showing associated pCR rates for Pembro, VC, and control arms by biomarker subset. Inset table shows pCR rates for HR+HER2−/Immune+vs. HR+HER2-/Immune− in the Pembro arm with Fisher's exact test p-value of association pCR ˜biomarker; as well, pCR rates and the association p-value are shown for HR+HER2−/DRD+vs. HR+HER2−/DRD− in the VC arm. In the barplots, # denotes patient subset too small to be evaluable (<5). FIG. 9E Association with pCR by RPS-5 (blue dots) vs. receptor subtype (red diamonds) by arm, where the y axis is −log(LR p) and the x axis is biascorrected mutual information. Blue (red) arrows and labels denote RPS-5 is more (less) predictive than receptor subtype. FIG. 9F and FIG. 9G Kaplan-Meier plots for Distant Recurrence-Free Survival (DRFS) by RPS-5 subtype, within patients who achieved pCR FIG. 9F and those with residual disease after chemo-targeted therapy FIG. 9G.

FIG. 10A-FIG. 10C. HER2-low example of adapting a response predictive subtyping schema to accommodate a new agent class. FIG. 10A 3-state HER2: Sankey plot showing relationship between HR status and Her2 low vs HER2=0 in the HER2-negative subset with HER2 IHC data available (585/742). FIG. 10B (sheet 29/30 and continuation sheet 30/33). Sankey diagram illustrating the relationship between receptor subtype and RPS-7 subtypes, with barplots to the right showing pCR rates by arm in each biomarker-defined subset. FIG. 10C In silico ‘thought experiment’ barplot showing pCR rates achieved in the control arm (black bar), experimental arms (orange bar); and estimated pCR rates if treatments were optimally assigned using receptor subtype (red bar) or RPS-7 (blue bar) in the population as a whole.

FIG. 11A-FIG. 11B. Mosaic plots showing the relationships between TN classifications by RPS-5 with two previously published TN subtyping schemas, the 4-class Brown/Bernstein classification (Burstein et al., 2015) FIG. 11A and the 7-class TNBCtype (Lehmann et al., 2011) FIG. 11B.

FIG. 12. 343 patients with HER2-negative BC with information on pCR and mRNA in 5 IO arms (Pembro: 69, Durva: 71, Pembro/SD101:72, Cemi: 60, Cemi/LAG3: 71) plus controls (Ctr: 179) were considered. 32 continuous markers including 30 immune (7 checkpoint genes, 14 immune cell, 3 T/B-cell prognostic, 1 TGFB and 5 tumor-immune) and ESR1/PGR and proliferation signatures, were assessed for association with pCR using logistic regression. p-values were adjusted using the Benjamini-Hochberg method (BH p<0.05).

### DETAILED DESCRIPTION

Patients to be Evaluated for Selection of Treatment

Patients that are evaluated for assignment to a treatment prediction subtype as described herein have Stage II or III breast cancer; with a minimum tumor size of 2.5 cm or greater by clinical exam or 2.0 cm or greater by imaging. Stage II or Stage III is determined in accordance with anatomic standards relating to tumor size, lymph node status, and distant metastasis. (as described by the American Joint Committee on Cancer). These patients include patients that have HER2 positive or negative tumors and HR positive or negative tumors. Stage II patients that are identified as low risk by a biomarker analysis panel, such as a MammaPrint® biomarker panel, do not typically undergo further assessment for assignment of a treatment prediction subtype, as chemotherapy or alternative therapeutic regimens have not been observed to provide further therapeutic benefit over surgery and radiation.

In some embodiments, alternative diagnostic tests are performed to determine that a Stage II breast cancer is low risk and therefore typically not assigned to a treatment prediction subtype. Such analysis of tumor profiles can employ tests such as those provided by Oncotype Dx (Genomic Health, Redwood City, CA), Prosigna (NanoString Technologies, Seattle WA), EndoPredict (Myriad Genetics, Salt Lake City, UT) and Breast Cancer Index (BCI) (Biotheranostics, Inc., San Diego, CA).

A breast cancer is considered to be HER2-negative (HER2−) if it does not detectably express HER2, whereas a breast cancer is determined to be HER2-positive (HER2+) if it does detectably express HER2. For this purpose, detectable expression is determined by evaluating protein expression, typically by immunohistochemistry fluorescent in situ hybridization.

Similarly, a breast cancer is considered to be estrogen receptor-negative (ER-negative or ER−) or progesterone receptor-negative (PR-negative or PR−) if it does not detectably express ER or PR, respectively, whereas a breast cancer is determined to be ER-positive (ER+) or PR-positive (PR+) if it does express ER or PR, respectively. For this purposes, detectable expression is determined by evaluating protein expression, typically by immunohistochemistry.

The term “HR+ refers to a breast cancer that is ER-positive and/or PR-positive.

For assignment to a treatment prediction subtype as described herein, breast cancers are also classified as luminal or basal molecular subtype. Basal breast cancers correlate best with triple negative (ER-negative, PR-negative, and HER2-negative) breast cancers (Rakha et al., 2009. Clin Cancer Res 15: 2302-2310; Carey et al., 2007. Clin Cancer Res 13: 2329-2334). Luminal-like cancers are ER-positive (Nielsen et al., 2004. Clin Cancer Res 10: 5367-5374), and HER2 positive cancers have a high expression of the HER2 gene (Kauraniemi and Kallioniemi. 2006. Endocr Relat Cancer 13: 39-49). The different molecular subtypes of breast cancer have different prognoses: luminal-like tumors have a more favorable outcome and basal-like and HER2 subgroups appear to be more sensitive to chemotherapy (Sorlie et al., 2001. Proc Natl Acad Sci USA 98: 10869-10874; Rouzier et al., 2005. Clin Cancer Res 11: 5678-5685; Liedtke et al., 2008. J Clin Oncol 26: 1275-1281; Krijgsman et al., 2012. Breast Cancer Res Treat 133: 37-47).

The MammaPrint® biomarker assay (Agendia) measures the activity of 70 genes to determine the 5-10-year relapse risk from women diagnosed with early breast cancer. The results are reported as either low-risk or high risk for developing distant metastases within 5 or 10 years after diagnosis. Extensive validation studies (Piccart et al., 2021. Lancet Oncol 22: 476-488; Cardoso et al., 2016. N Engl J Med 375: 717-729; Drukker et al., 2013. Int J Cancer 133: 929-936; Bueno-de-Mesquita et al., 2007. Lancet Oncol 8: 1079-1087; van de Vijver et al., 2002. New Engl J Med 34: 1999-2009) have demonstrated the predictive value of the assay. The assay is described in WO2002103320, which is incorporated by reference. According to WO2002103320, a MammaPrint® test (also termed “Amsterdam gene signature test” or MP) is based on the expression levels of at least 5 genes from a total of 231 indicated in Table 3. Genes that are included in the 70 genes MP signature are PALM2-AKAP2, ALDH4A1, AP2B1, BC3, C16 orf95, CAPZB, CCNE2, CDC42BPA, CDCA7, CENPA, CMC2, COL4A2, DCK, DHX58, DIAPH3, DTL, EBF4, ECI2, ECI2, ECT2, EGLN1, ESM1, EXT1, FGF18, FLT1, GMPS, GNAZ, ADGRG6, GPR180, GRHL2, GSTM3, SERF1A, HRASLS, IGFBP5, JHDMID-AS1, LIN9, LPCAT1, MCM6, MELK, MIR210HG, MMP9, MS4A7, MS4A14, MSANTD3, MTDH, NDC80, NMU, NUSAPI, ORC6, OXCT1, PITRM1, PRC1, QSOX2, RAB6B, RFC4, RTN4RL1, RUNDC1, SCUBE2, SLC2A3, SMIM5, STK32B, TGFB3, TMEM65, TMEM74B, TSPYL5, UCHL5, WISP1 and ZNF385B.

Prediction Subtypes

Described herein are methods of classifying breast cancer tumors for assignment to an RPS as described herein. The method comprises analysis of tumors to interrogate various biological pathways in addition to HER2 and HR signaling pathways. As detailed herein, tumors are assigned to a response-predictive biological phenotype by considering promising treatments (e.g., immunotherapy, dual-HER2, and platinum-based) and basic cancer biology (e.g. proliferation and DNA repair deficiency).

For purposes of this disclosure, patients are considered Immune-positive (Immune+) if their immune-tumor state, also referred to herein as immune profile, is such that they are likely to respond to immunotherapy based on analysis of panels of immune pathway markers, e.g., those provided in Table A, as described herein; and are considered DNA repair deficient/platinum-responsive (DRD+) if response to a platinum agent with or without PARP-inhibition is likely. As biomarkers representing the same biology are correlated and can be subtype-specific, multiple immune and DRD markers can be used to implement these biological phenotypes and perform similarly. Furthermore, as alternative biomarkers come available, they can be substituted for biomarker panels described herein.

The present disclosure thus provides various classifications for selecting a therapy based on assigning the patient to a response prediction subtype classification based on analysis of biomarker panels comprising immune response genes, DNA repair gene, HER2 status, and assignment of Basal-type or Luminal-type status. In some embodiments, methods of assigning a patient to a response prediction subtype comprises assigning the patient to one of five classifications: HER2−/Immune−/DRD−, HER2−/Immune−/DRD+, HER2−/Immune+, HER2+/Blueprint-HER2 or Blueprint-Basal, and HER2+/Blueprint-Luminal.

Determination of Luminal, Basal, HER2-type

As is used herein, the term “BluePrint®” (U.S. Pat. Nos. 9,175,351; 10,072,301; Krijgsman et al., 2012. Br Can Res Treat 133: 37-47) refers to a molecular subtyping test, analyzing the activity of 80 genes to stratify breast cancer into one of three subtypes: luminal-, basal- or HER2-type. Alternatively, the PAM50 classifier (Parker, et al., JCO 27, 1160-1167 (2009) can be employed. In some embodiments, “HER2-ness” is assessed using any test classifying a tumor with either cell membrane presence of HER2 protein and functional activity of the pathway, e.g., using BluePrint® or PAM50 classifier. In some embodiments assignment of a tumor as a luminal-type, basal-type or HER2-type employ the 80-gene BluePrint® panel, or a subset thereof, e.g., as described in US Patent Application Publication No. 20160115552. As described in U.S. Pat. Nos. 9,175,351 and 10,072,301, BluePrint® analysis involves determining RNA expression levels of at least adrenomedullin (ADM), Coiled-Coil Domain Containing 74B (CCDC74B), Moesin (MSN), Thrombospondin Type 1 Domain Containing 4 (THSD4), Perl-Like Domain Containing 1 (PERLD1) and Synaptonemal Complex Protein 3 (SYCP3), of Neuropeptide Y Receptor Y1 (NPY1R), SRY-Box Transcription Factor 11 (SOXI1), ATP Binding Cassette Subfamily C Member 11 (ABCC11), Proline Rich 15 (PRR15) and Erb-B2 Receptor Tyrosine Kinase 2 (HER2; ERBB2), or of a combination thereof. The 80 genes included in the BluePrint® test are indicated in Table 4.

Determination of Immune Status

In the present disclosure, “Immune+” and” Immune −” means that the patient with a tumor of such status has a likelihood to benefit from/respond to immune modulating therapy (if immune+) or not likely (if immune−). As used herein, determining the “immune status” or “immune profile” of a tumor refers to classifying a breast cancer tumor as having a positive or negative immune response profile for responding to an immunotherapy treatment. Determining the immune status comprises analyzing one or more biomarker panels comprising immune response genes to determine whether or not a patient has an immune response profile value (e.g., based on expression pattern, e.g., number of immune response genes expressed and/or level of expression), that is associated with an increased likelihood of a high pCR to a treatment that targets one or more genes that regulate T-cell, B-cell, dendritic cell, or natural killer (NK) cell immune functions, e.g., a checkpoint inhibitor therapy, compared to alternative therapies, such as a therapy that targets DNA repair defects. As used herein a “high” or “highest” pCR refers to a comparison of pCR rates among therapy options. Thus, for example, for a HER2−/Immune+ breast cancer, a therapy such as Pembro is considered to have the highest pcR rate relative to other therapies that target DNA repair pathways, the AKT pathway, standard chemotherapy, etc.

In some embodiments, an immune response profile value associated with an increased likelihood of a pCR is considered positive when it reaches or exceeds a threshold value. Similarly, an immune response profile is considered negative when it is below the threshold value. In some embodiments, an immune response profile is determined for one or more immune response biomarker panels designated as follows and shown in Table A.
Module5_TcellBcell (PMID:24516633: Wolf et al, PLOS ONE Feb. 7, 2014, 9(2), e883019, pages 1-16);
ICS5 (PMID:24172169; Yau et al, Bresat Cancer Res. 2013; 15(5): R103);
B-cells (PMID:28239471, Danaher et al, J. Immunother Cancer 2018 Feb. 21; 5:18)
Dendritic cells (PMID:28239471, Danaher et al, 2018, supra);
Mast cells (PMID:28239471, Danaher et al, 2018, supra);
STAT1_sig (PMID:19272155, Rody et al, Breast Cancer Res. 2009:11(2): R15, Epub Mar. 9, 2009);
Chemokinel2 (PMID:21703392, Coppola et al., Am J. Pathol. 2011 July:179)1):37-45);
Module 3_IFN (PMID:24516633, Wolf et al, 2014, supra).

The expression score can be determined using various methods. In some embodiments, continuous biomarkers can be dichotomized using a subtype-specific cross-validation procedure to optimize performance. For example, a cross-validation procedure can be applied to select endpoints associated with pCR in a selected treatment arm of the trial to identify cutoff points for biomarker positively. Logistic models can be employed to assess association with response. For example, in the examples described herein, a cutpoint was selected as ‘optimal’ if: (1) it was selected as optimal>100 times in the training set; (2) p<E-15 in the test sets (combined using the logit method (Dewey, 2018)); and (3) the prevalence is reasonably balanced.

One of skill understand that alternative bioinformatics algortihms can also be employed to determine an expression score. Thus, classification of a positive or negative immune response profile based on gene expresson profiling of an immune response panel can be performed by a number of statistical techniques including, but not limited to, Markov clusterin, multi-state semi-Markov models, Cox Proportional Hazards models, shrinkage based methods, tree based methods, Bayesian methods, kernel based methods and neural networks. For example, established statistical algorithms and methods useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (LogReg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others.

In some embodiments, an immune response profile may be determined by evaluating expression of a subset of genes in an immune response panel and/or by assessing other genes that are indicators of immune pathway activation or suppression. For example, determining an immune response profile may comprise analyzing expression of a subset of at least five or more, or ten or more or fifteen or more, or twenty or more genes of a Module5_TcellBcell panel; and/or three or more or five or more genes of a STAT1 panel or chemokine 12 panel (see, Table A). In some embodiments, one or more genes identified as playing a role in the pathways/cell-types indicated in the first column of Table A may be added to the panel or substituted in the panel.

TABLE A

| | | Scoring method* |
|---|---|---|
| | | *starting with |
| | | normalized and |
| | | combined |
| | | transcriptome and |
| Biomarkers | Genes/proteins | RPPA data |
| Module5_TcellBcell | IGSF6, LILRB2, BTN3A3, UBD, CXCL13, GNLY, CXCR6, CTSC, | 1) Mean center, 2) |
| | HCP5, PIM2, SP140, CCR7, CTSS, CYBB, FCN1, TFEC, SEL1L3, | take modified inner |
| | FYB, GBP1, LAMP3, ADAMDEC1, GPR18, ICOS, GPR171, | product with |
| | GZMH, GZMB, GZMK, BIRC3, IFNG, IL2RG, IL15, IDO1, | centroid as published |
| | CXCL10, IRF1, ISG20, ITK, LAG3, LCK, LYN, CXCL9, NKG7, | and described below |
| | TRAT1, MGC29506, PLAC8, POU2AF1, CRTAM, SLAMF8, | (though averaging |
| | PSMB9, PTPN7, SLAMF7, BCL2A1, TNFRSF17, CCL5, CCL8, | would yield similar |
| | CCL13, CCL18, CCL19, CXCL11, SELL, SAMSN1, RTP4, CLEC7A, | results), 3) Z-score |
| | TAP1, WARS, PLA2G7, ZBED2, NPL, RUNX3, VNN2, CD3G, | |
| | IL32, CD8B, CD19, CD86, AIM2, CD38, CYTIP, LOC96610, | |
| | CD69, CD79A | |
| ICS5 | CXCL13, CLIC5, HLA-F, TNFRSF17, XCL2 | 1) Mean center, 2) |
| | | average over genes, |
| | | 3) Z-score |
| B_cells | BLK, CD19, FCRL2, KIAA0125, MS4A1, PNOC, SPIB, TCL1A, | 1) Average over |
| | TNFRSF17 | genes, 2) mean |
| | | center, 3) Z-score |
| Dendritic_cells | CCL13, CD209, HSD11B1 | 1) Average over |
| | | genes, 2) mean |
| | | center, 3) Z-score |
| Mast_cells | CPA3, HDC, MS4A2, TPSAB1, TPSB2 | 1) Average over |
| | | genes, 2) mean |
| | | center, 3) Z-score |
| STAT1_sig | TAP1, GBP1, IFIH1, PSMB9, CXCL9, IRF1, CXCL11, CXCL10, | 1) Mean center, 2) |
| | IDO1, STAT1 | average over genes, |
| | | 3) Z-score |
| Chemokine12 | CCL2, CCL3, CCL4, CCL5, CCL8, CCL18, CCL19, CCL21, CXCL9, | 1) Mean center, 2) |
| | CXCL10, CXCL11, CXCL13 | average over genes, |
| | | 3) Z-score |
| Module3_IFN | IFI44, IFI44L, DDX58, IFI6, IFI27, IFIT2, IFIT1, IFIT3, CXCL10, | 1) Mean center, 2) |
| | MX1, OAS1, OAS2, OAS3, HERC5, SAMD9, HERC6, DDX60, | take modified inner |
| | RTP4, IFIH1, STAT1, TAP1, OASL, RSAD2, ISG15 | product with |
| | | centroid as published |
| | | and described below |
| | | (though averaging |
| | | would yield similar |
| | | results), 3) Z-score |

In some embodiments, determination of Immune+ or Immune− status comprises evaluating Module 5 TcellBcell, B_cells, Dendritic_cells, STAT1_sig, Mast Cell, and chemokine 12 biomarker panels.

Determination of DNA Repair Deficiency (DRD) Status

In the present disclosure, “DRD+” and” DRD −” means that a patient with a tumor of such status has a likelihood to benefit from/respond to a therapy that targets a DNA repair defict (if DRD+) or not likely (if DRD−). As used herein, determining the “DRD status” or “DRD profile” of a tumor refers to classifying a breast cancer tumor as having a positive or negative DRD response profile for responding to DRD-targeted treatment. Determining the DRD status comprises analyzing one or more biomarker panels comprising genes indicative of DNA repair status to determine whether or not a patient has a DRD response profile value (e.g., based on expression pattern, e.g., number of DRD genes expressed and/or level of expression), that is associated with an increased likelihood of a high pCR to a treatment that targets DNA repair defects, compared to alternative therapies, such as immunotherapies.

In some embodiments, a DRD response profile value associated with an increased likelihood of a pCR is considered positive when it reaches or exceeds a threshold value. Similarly, DRD response profile is considered negative when it is below the threshold value. In some embodiments, a VCpred_TN panel is employed for tumors that are triple-negative, i.e., ER⁻/PR⁻/HER2⁻. In some embodiments, a DRD response profile is determined for one or more DRD biomarker panels designated as follows and shown in Table B.
PARPi7 (PMID: 22875744, Daemen et al, Breast Cancer Res Treat 2012, 135(2):505-517, 2012; and PMID: 28948212, Wolf et al., NPJ Breast Cancer 2017 August 25; 3:31, eCollectoin 2017);
PARPi7_plus_MP2, Genes in PARPi7+Genes in MP index (PMID 28948212, Wolf et al., 2017, supra);
VCpred_TN (described herein)

The expression score can be determined using various methods. In some embodiments, continuous biomarkers can be dichotomized using a subtype-specific cross-validation procedure to optimize performance. For example, a cross-validation procedure can be applied to select endpoints associated with pCR in a selected treatment arm of the trial to identify cutoff points for biomarker positively. Logistic models can be employed to assess association with response. For example, in the examples described herein, a cutpoint was selected as ‘optimal’ if: (1) it was selected as optimal>100 times in the training set; (2) p<E-15 in the test sets (combined using the logit method (Dewey, 2018)); and (3) the prevalence is reasonably balanced.

One of skill understand that alternative bioinformatics algortihms can also be employed to determine an expression score. Thus, classification of a positive or negative DRD response profile based on gene expresson profiling of a DRD response panel can be performed by a number of statistical technique as detailed herein in the section regarding analysis of immune response panel expression profiles.

In some embodiments, a DRD response profile may be determined by evaluating expression of a subset of genes in a DRD response panel. For example, determining a DRD response profile may comprise analyzing expression of a subset of at least three or more of a PARPi7 panel; and/or at least five or more genes of a Mammaprint (MP) index panel. In some embodiments, one or more other biomarkers indicative of DNA Repair status can be evaluated in addition to those listed in a panel below. In some embodiments, an alternative biomarker indicative of DNA Repair status can substitute for one of the biomarkers below.

TABLE B

| | | |
|---|---|---|
| PARPi7 | Prediction genes: BRCA1, | 1) divide each PARPi-7 predictor gene level (not centered) |
| | CHEK2, MAPKAPK2, | by the geometric mean of the normalization genes, 2) |
| | MRE11A, NBN, TDG, XPA; | log2-transform each ratio and median center, 3) calculate |
| | Normalization genes: | score as Weights*(Genes − Boundaries), using Weights = |
| | RPL24, ABI2, GGA1, E2F4, | (−0.5320, 0.5806, 0.0713, −0.1396, −0.1976, −0.3937, −0.2335) |
| | IPO8, CXXC1, RPS10 | and Boundaries = (−0.0153, −0.006, 0.0031, −0.0044, |
| | | 0.0014, −0.0165, −0.0126), 4) standardize to sd = 1 |
| PARPi7_plus_MP2 | Genes in PARPi7 + Genes in | 1) PARPi7 + MP_index_adj*(−1), 2) Z-score |
| | MP_index | |
| VCpred_TN | CXCL13, BRCA1, APEX1, | 1) mean center, 2) calculate weighted average = |
| | FEN1, CD8A, SEM1, APEX2, | (13.60*CXCL13 − 6.48*BRCA1 + 6.41*APEX1 + 5.32*FEN1 + |
| | RNMT, CCR7, H2AFX, | 4.85*CD8A − 4.84*SEM1 + 4.78*APEX2 − 4.60*RNMT + |
| | POLD3, PRKDC, C1QA, | 4.51*CCR7 + 3.99*H2AFX + 3.88*POLD3 − 3.49*PRKDC + |
| | CLIC5, RAD51, DDB2, SPP1, | 3.48*C1QA + 3.33*CLIC5 − 3.24*RAD51 + 3.10 *DDB2 − |
| | OLD2 POLB, LIG1, GTF2H5, | 2.83*SPP1 − 2.80 *POLD2 − 2.80*POLB + 2.72*LIG1 − |
| | PMS2, LY9, SHPRH | 2.67*GTF2H5 − 2.63*PMS2 + 2.60*LY9 − 2.34*SHPRH + |
| | | 6.27*ARAF), 3) Z-score |

Expanded Predictor Subtype Classification

In some embodiments, a response predictor subtype may comprise seven classifications, in which HER2+ subtypes are further classified based on “HER2-ness”. In this schema, HER2 levels of breast cancers are assigned as HER2−0, HER2-low, or HER2+. “HER2-ness” can be assessed based on one or more of the following ERBB2 evaluations:
HER2_Index, (PMID: 21814749, Krijgsman et al, Breast Cancer Res. Treat 133:37-47, 2012)
Mod7_ERBB2 (PMID: 24516633, Wolf et al, PLoS One 9: e88309, 2014)
EGFR.Y1173 (PMID: 32914002, Wulfkuhle et al, JCO Precis Oncol 2: PO.18.0024, 2018)
EGFR.Y1173 (PMID: 32914002, Wulfkuhle et al, 2018, supra)

TABLE C

| | | |
|---|---|---|
| HER2_Index | ERBB2, GRB7, PERLD1, | Z-score HER2 index values from BluePrint (Agendia). |
| (HER2_type) | SYCPB | Scoring algorithm proprietary but based on nearest |
| | | centroid method in publication |
| Module7_ERBB2 | ERBB2, GRB7, STARD3, | 1) Mean center, 2) take modified inner product with |
| | PGAP3 | centroid as published and described in examples, 3) |
| | | Z-score |
| ERBB2 Y1248 | phospho-protein ERBB2 | Z-score values |
| | Y1248 | |
| EGFR Y1173 | phospho-protein ERBB2 | Z-score values |
| | Y1248 | |

Accordingly, one of skill can further classify a tumor as HER2−0/HER2-low or HER2+.

Determining Expression Levels of Genes in a Panel

The level of RNA, typically mRNA transcripts encoded by a gene, in an RNA sample from a breast cancer sample obtained from a patient as described above can be detected or measured by a variety of methods including, but not limited to, an amplification assay, sequencing assay, or a hybridization assay such as a microarray chip assay. As used herein, “amplification” of a nucleic acid sequence has its usual meaning, and refers to in vitro techniques for enzymatically increasing the number of copies of a target sequence. Amplification methods include both asymmetric methods in which the predominant product is single-stranded and conventional methods in which the predominant product is double-stranded. The term “microarray” refers to an ordered arrangement of hybridizable elements, e.g., gene-specific oligonucleotides, attached to a substrate. Hybridization of nucleic acids from the sample to be evaluated is determined and converted to a quantitative value representing relative gene expression levels.

Non-limiting examples of methods to evaluate levels of RNA include amplification assays such as quantitative RT-PCR, digital PCR, isothermal amplification methods such as qRT-LAMP, strand displacement amplification, ligation chain reaction, or oligonucleotide elongation assays. In some embodiments, multiplexed assays, such as multiplexed amplification assays are employed.

In some embodiments, expression level is determined by sequencing, e.g., using massively parallel sequencing methodologies. For example, RNA-Seq can be employed to determine RNA expression levels. Other sequencing methods include example, R, sequencing-by-synthesis, paired-end sequencing, single-molecule sequencing, nanopore sequencing, pyrosequencing, semiconductor sequencing, sequencing-by-ligation, sequencing-by-hybridization, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing.

Typically measured RNA values are normalized to account for sample-to-sample variations in RNA isolation and the like. Methods for normalization are well known in the art. In some embodiments, normalized values may be obtained using a reference level for one or more of control gene; or exogenous RNA oligonucleotides. A control value for normalization of RNA values can be predetermined, determined concurrently, or determined after a sample is obtained from the subject. Thus, for example, the reference control level for normalization can be evaluated in the same assay or can be a known control from one or more previous assays.

In alternative embodiments, expression of a panel of genes is determined by analyzing levels of protein expressed by the gene. Protein levels can be detected by immunoassay or use of binding agents that bind to a protein of interest, e.g., aptamers. In some embodiments, protein modification may be assessed, e.g., phosphorylation status of biomarker proteins that are phosphorylated/desphosphorylated in various kinase pathways can be assessed.

Classification methods described herein may be totally or partially performed with a computer system including one or more processors, which can be configured to perform the steps. Thus, some embodiments are directed to computer systems configured to perform the steps of any of the methods described herein, potentially with different components performing a respective step or a respective group of steps. Typically, the computer will be appropriately programmed for receipt and storage of the data from the device, as well as for analysis and reporting of the data gathered. Results can be cast in a transmittable form of information that can be communicated or transmitted to other individuals, e.g., researchers or physicians, or patients. Such a form can vary and can be tangible or intangible. The result in the individual tested can be embodied in descriptive statements, diagrams, charts, images or any other visual forms. For example, statements regarding levels of gene expression and levels of protein may be useful in indicating the testing results. Statements and/or visual forms can be recorded on a tangible media or on an intangible media and transmitted. In addition, the result can also be recorded in a sound form and transmitted through any suitable media, e.g., analog or digital cable lines, fiber optic cables, etc., via telephone, wireless mobile phone, internet phone and the like. All such forms (tangible and intangible) would constitute a “transmittable form of information”. Thus, the information and data on a test result can be produced anywhere and transmitted to a different location.

Received data, e.g., immune and DRD profile data, can provide immune status and DNA Repair deficiency status to allow assignment of a breast cancer to a response predictor subtype in conjunction with data for hormone receptor and HER2 status. Additional data that can be transmitted/received includes includes HER2 status, hormone status, basal or luminal classification, and/or “HER2ness”. Accordingly, patients can be classified for DNA-Repair-Deficiency sensitivity (DRD+ or −) and Immune-modulation sensitivity (Immune+ or −). Receptor subtypes HR+/HER2− and TN breast cancers are classified to HER2−/Immune-/DRD−, HER2−/Immune+(including both DRD+ or − status), and HER2−/Immune−/DRD+ classes. In addition, Receptor Subtypes HR−/HER2+ and HER+/HER2+ can be reclassified by the Response Predictive Subtypes into HER2+/BluePrint-HER2type or Basaltype, and HER2+/BluePrint-luminal type.

Selection of Treatment Regimens

Selection of a treatment is based on comparison of pCR rates for various treatment protocols as described in the section “ANALYSIS OF PATIENT DATA THAT IDENTIFIED RESPONSE PREDICTOR SUBTYPES” to assign a breast cancer tumor to a response predictor subtype. The treatment that shows the highest pCR for tumors categorized into each of the subtypes classifications, e.g., HER2−/Immune−/DRD−, HER2−/Immune−/DRD+, HER2−/Immune+, HER2+/Blueprint-HER2 or Basal, and HER2+/Blueprint-Luminal, is typically selected as a recommended therapy. However, one of skill understands that other considerations, such as toxicity, are taken into account when ultimately selecting a therapy for a patient.

As is used herein, the term “combination” refers to the administration of effective amounts of compounds to a patient in need thereof. Said compounds may be provided in one pharmaceutical preparation, or as two or more distinct pharmaceutical preparations. Said compounds may be administrated simultaneously, separately, or sequentially to each other. When administered as two or more distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician. Said protocol may include daily administration for 1-30 days, such as 2 days, 10 days, or 21 days, followed by period of 1-14 days, such as 7 days, in which no compound is administered.

As described herein, a therapy to treat the breast cancer can be selected based on the response predictive subtype. In some embodiments, a checkpoint inhibitor therapy, e.g., a PD1/PDL1 checkpoint inhibitor therapy, is selected for a breast cancer assigned to the HER2−/Immune+ subtype. In some embodiments, a dual-anti-HER2 therapy, e.g., anti-HER2 therapeutic antibodies, is selected for a breast cancer assigned to the HER2+ that are not luminal subtype. In some embodiments, a DNA repair therapy, such as a platinum-based therapy or a PARP inhibitor is selected as a therapeutic agent for a breast cancer assigned to a HER2−/Immune−/DRD+ subtype. In some embodiments, a combination therapy including an AKT inhibitor or AKT pathway inhibitor is selected for a breast cancer assigned to the HER2+/BP-Luminal subtypes. In some embodiments, a neoadjuvant endocrine therapy is selected for a HR+ breast cancer assigned to the HER2−/Immune−/DRD− subtype.

Illustrative treatments for each of the categories are provided below. In this example treatment schema, the HER2−/DRD−/Immune− is split based on either HR+ or TN (their origin). Thus, for example, for the RPS5 5 subtypes, 6 sets of 2 regimens are:
HER2−/DRD−/Immune−/HR+: paclitaxel or paclitaxel plus AKTi
HER2−/DRD−/Immune−/TN: carboplatin+paclitaxel or carboplatin
   +paclitaxel+PD1/PDL1 inhibitor
HER2−/Immune+: PD-1/PDL-1 inhibitor+paclitaxel or
   PD-1/PDL-1 inhibitor+paclitaxel+carboplatin
HER2−/Immune−/DRD+: carboplatin+paclitaxel or
   carboplatin+paclitaxel+PD1/PDL1 inhibitor
HER2+/BP-HER2-type or Basal-type: paclitaxel+trastuzumab+pertuzumab (THP) or
   paclitaxel+carboplatin+trastuzumab+
   pertuzumab (TCHP)
HER2+/BP-luminal-type: paclitaxel+trastuzumab+pertuzumab (THP), or
   paclitaxel+trastuzumab+AKTi.

In some embodiments, a patient categorized as having a HER2−/DRD−/Immune−/TN subtype breast cancer is not administered a PD1/PDL1 inhibitor. In some embodiments, HER2− can be further subdivided into HER2−0 and HER2-low groups, for therapies that specifically target HER2-low tumors.

The invention provides a method of typing a Stage II or Stage III breast cancer, comprising i) determining the breast cancer's HER2 status; ii) determining a molecular subtype, for example by determining the breast cancer's BluePrint status, i.e. assignment of the breast cancer BluePrint HER2+, BluePrint Basal or BluePrint Luminal subtype; iii) determining the breast cancer's immune response profile for responding to an immunotherapy treatment, wherein a positive immune response profile is assigned by determining that the expression pattern of at least one panel of immune status genes reaches or exceeds a threshold that is associated with a high pathology complete response (pCR) rate for patients treated with an immune pathway-targeted therapy compared to patients treated with therapies that do not target the immune response; and a negative immune response profile is assigned by determining that the expression pattern is lower than the threshold; iv) determining the breast cancer's DNA Repair Defect (DRD) profile for responding to a DNA repair treatment, wherein a positive DRD response profile is assigned by determining that the expression pattern of at least one panel of DRD status reaches or exceeds a threshold that is associated with a high pathology complete response (pCR) rate for patients treated with a DNA repair-targeted therapy compared to patients treated with therapies that do not target DNA repair; and a negative DRD response response profile is assigned by determining that the expression pattern is lower than the threshold; and v) assigning the breast cancer to a response predictor subtype selected from the group consisting of HER2−/Immune−/DRD−, HER2−/Immune−/DRD+, HER2−/Immune+, HER2+/BP-HER2-type or Basal-type, and HER2+/BP-Luminal.-type, thereby typing the breast cancer for an anticipated response to a therapeutic treatment. More specifically, the breast cancer response predictor subtypes HER2−/Immune−/DRD−, HER2−/Immune−/DRD+, HER2−/Immune+, HER2+/BP-HER2-type or Basal-type, and HER2+/BP-Luminal.-type, are predicted to respond to the following thereapeutic treatments: dual-anti-HER2 therapy, DNA repair targeted therapy, immune therapy, dual-anti-HER2 therapy and a combination therapy comprising an AKT pathway-inhibitor, respectively.

The term “typing of a breast cancer”, as is used herein, refers to the classification of a breast cancer based on the expression levels of genes, which may assist in the prediction of a response to a therapeutic treatment.

The invention further provides a therapeutic treatment option for use in the treatment of the a breast cancer that is typed as sHER2−/Immune−/DRD−, HER2−/Immune−/DRD+, HER2−/Immune+, HER2+/BP-HER2-type and/or Basal-type, and HER2+/BP-Luminal.-type.

As such, the invention provides a DNA repair targeted therapy for use in a method of treating a Stage II or Stage III breast cancer, wherein said cancer is typed as HER2−/Immune−/DRD+. Said DNA repair targeted therapy preferably is or comprises a platinum based therapy and/or a PARP inhibitor. A preferred DNA repair targeted therapy for a breast cancer typed as subtype HER2−/Immune−/DRD+ comprises a combination of carboplatin and paclitaxel, optionally further comprising a PD1/PDL1 inhibitor.

The invention further provides an immune therapy for use in a method of treating a Stage II or Stage III breast cancer, wherein said cancer is typed as HER2−/Immune+. Preferably, said immune response therapy is or comprises a immune check point inhibitor such as a PDL1/PD1 checkpoint inhibitor. Most preferably, said immune response therapy comprises a combination of an immune check point inhibitor such as a PDL1/PD1 checkpoint inhibitor with paclitaxel, optionally further comprising carboplatin.

The invention further provides a dual-anti-HER2 therapy for use in a method of treating a Stage II or Stage III breast cancer, wherein said cancer is typed as HER2+/BP-HER2-type and/or Basal-type. A preferred dual-anti-HER2 therapy comprises a combination of paclitaxel, trastuzumab and pertuzumab (known as “THP”) or a combination of paclitaxel, carboplatin, trastuzumab and pertuzumab (known as “TCHP”).

The invention further provides a combination therapy for use in a method of treating a Stage II or Stage III breast cancer, wherein said cancer is typed as HER2+/BP-Luminal-type. Preferably said combination therapy comprises a combination of paclitaxel, trastuzumab and pertuzumab (known as “THP”) or a combination of paclitaxel, trastuzumab and a AKT inhibitor. Said combination therapy optionally comprises an AKT pathway-inhibitor

The invention further provides a neaoadjuvant endocrine therapy for use in a method of treating a Stage II or Stage III breast cancer, wherein said cancer is typed as HER2−/Immune−/DRD−.

In some embodiments, an immune therapy is a checkpoint inhibitor selected to treat a breast cancer. In some embodiments, the checkpoint inhibitor inhibits PD-1/PD-L1 interaction. In some embodiments, the immune checkpoint inhibitor is an inhibitor of PD-L1. In some embodiments, the immune checkpoint inhibitor is an inhibitor of PD-1. In some embodiments, a breast cancer may be classified as an Immune+ subtype and the patient is administered an alternative checkpoint inhibitor such as a CTLA-4, PDL1, ICOS, PDL2, IDO1, IDO2, PD1, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137, CD160, CD39, VISTA, TIGIT, CGEN-15049, 2B4, CHK 1, CHK2, A2aR, or B-7 family ligand inhibitor, or a combination thereof. In some embodiments, the checkpoint inhibitor is pembrolizumab. Furthermore, many other immune response pathway therapies targeting alternative pathways will be useful for treatment of breast cancers assigned to the Immune+ subtype.

Suitable immune checkpoint inhibitors are CTLA-4 inhibitors such as antibodies, including ipilimumab (Bristol-Myers Squibb) and tremelimumab (MedImmune); PD1/PDL1 inhibitors such as antibodies, including pembrolizumab (Merck), sintilimab (Eli Lilly and Company), tislelizumab (BeiGene), toripalimab (Shangai Junshi Biosciense Company), spartalizumab (Novartis), camrelizumab (Jiangsu HengRui Medicine C), nivolumab and MDX-1105 (Bristol-Myers Squibb), pidilizumab (Medivation/Pfizer), MEDIO680 (AMP-514; AstraZeneca), cemiplimab (Regeneron) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), KN035 (Jiangsu Alphamab Biopharmaceuticals Company), Cosibelimab (CK-301; Checkpoint Therapeutics), BMS-936559 (Bristol-Myers Squibb), BMS-986189 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl]methyl]piperidine-2-carboxylic acid), BMS202 (PD-1/PD-L1 Inhibitor 2; WO2015034820; N-[2-[[[2-methoxy-6-[(2-methyl[1,1′-biphenyl]-3-yl)methoxy]-3-pyridinyl]methyl]amino]ethyl]-acetamide), PD-1/PD-L1 Inhibitor 3 (WO/2014/151634; (3S,6S,12S,15S,18S,21S,24S,27S,30R,39S,42S,47aS)-3-((1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13), CA-170 (Curis) and ladiratuzumab vedotin (Seattle Genetics).

In some embodiments, a dual-anti-HER2 therapy is selected for a breast cancer assigned to the HER2−/Immune+ subtype. Such therapies target EGFR and HER2. In some embodiments, the therapeutic agent is neratinib. In some embodiments the therapeutic agent is lapatinib. In some embodiments, a dual-anti-HER2 therapy comprises treatement with trastuzumab (optionally as an antibody-drug conjugate such as trastuzumab deruxtecan) or pertuzumab (optionally as an antibody-drug conjugate such as pertuzumab emtansine (T-DM1)), in combination with lapatinib, tucatinib or neratinib. In some embodiments, a dual-anti-HER2 therapy is selected for a breast cancer assigned to the HER2+ that are not luminal subtype.

Therapies that target the AKT pathway are known. Illustrative agents are described, e.g., by Martorana et al, Front. Pharmacol. Vol 12, Article 66223, 2021 (doi: 10.3389/fphar.2021.662232), which is incorporated by reference. In some embodiments, an agent that targets the AKT pathway is an AKT inhbitior that interacts with AKT to inhibit activity. An AKT inhibitor (AKTi) may be selected from miransertib (3-[3-[4-(1-aminocyclobutyl)phenyl]-5-phenylimidazo[4,5-b]pyridin-2-yl]pyridin-2-amine; ARQ 092, Merck & Co. Inc), vevorisertib (N-[1-[3-[3-[4-(1-aminocyclobutyl)phenyl]-2-(2-aminopyridin-3-yl) imidazo[4,5-b]pyridin-5-yl]phenyl]piperidin-4-yl]-N-methylacetamide; ARQ 751, Merck & Co. Inc), MK-2206 (8-[4-(1-aminocyclobutyl)phenyl]-9-phenyl-2H-[1,2,4]triazolo[3,4-f][1,6]naphthyridin-3-one; Merck & Co. Inc), perifosine ((1,1-dimethylpiperidin-1-ium-4-yl) octadecyl phosphate, KRX-0401, Keryx Biopharmaceuticals), ATP competitive inhibitors, such as ipatasertib (Roche; (2S)-2-(4-chlorophenyl)-1-[4-[(5R,7R)-7-hydroxy-5-methyl-6,7-dihydro-5H-cyclopenta[d]pyrimidin-4-yl]piperazin-1-yl]-3-(propan-2-ylamino)propan-1-one;), uprosertib (GlaxoSmithKline; (N-[(2S)-1-amino-3-(3,4-difluorophenyl)propan-2-yl]-5-chloro-4-(4-chloro-2-methylpyrazol-3-yl)furan-2-carboxamide), capivasertib (AstraZeneca; 4-amino-N-[(1S)-1-(4-chlorophenyl)-3-hydroxypropyl]-1-(7H-pyrrolo[2,3-d]pyrimidin-4-yl)piperidine-4-carboxamide) and afuresertib (N-[(2S)-1-amino-3-(3-fluorophenyl)propan-2-yl]-5-chloro-4-(4-chloro-2-methylpyrazol-3-yl)thiophene-2-carboxamide).

PARP inhibitors are also known. Illustrative agents are described e.g., by Rose et al, Frontiers in Cell land Developmental Biol. Vol 8, Article 564601, 2020 (doi 10.3389/fcell.2020.564601), which is incorporated by reference.

A PARP inhibitor may be selected from olaparib (3-aminobenzamide, 4-(3-(1-(cyclopropanecarbonyl)piperazine-4-carbonyl)-4-fluorobenzyl)phthalazin-1(2H)-one; AZD-2281; AstraZeneca), rucaparib (6-fluoro-2-[4-(methylaminomethyl)phenyl]-3,10-diazatricyclo[6.4.1.04,13]trideca-1,4,6,8(13)-tetraen-9-one; Clovis Oncology, Inc.); niraparib tosylate ((S)-2-(4-(piperidin-3-yl)phenyl)-2H-indazole-7-carboxamide hydrochloride; MK-4827; GSK); talazoparib (11S,12R)-7-fluoro-11-(4-fluorophenyl)-12-(2-methyl-1,2,4-triazol-3-yl)-2,3,10-triazatricyclo[7.3.1.05,13]trideca-1,5(13),6,8-tetraen-4-one; BMN-673; Pfizer); fluzoparib (4-[[4-fluoro-3-[2-(trifluoromethyl)-6,8-dihydro-5H-[1,2,4]triazolo[1,5-a]pyrazine-7-carbonyl]phenyl]methyl]-2H-phthalazin-1-one; Jiangsu Hengrui Pharmaceuticals);veliparib (2-[(2R)-2-methylpyrrolidin-2-yl]-1H-benzimidazole-4-carboxamide dihydrochloride benzimidazole carboxamide; ABT-888; Abbvie); pamiparib (2R)-14-fluoro-2-methyl-6,9,10,19-tetrazapentacyclo[14.2.1.02,6.08,18.012,17]nonadeca-1(18),8,12(17),13,15-pentaen-11-one; BGB-290; BeiGene); CEP-8983, and CEP 9722, a small-molecule prodrug of CEP-8983, a 4-methoxy-carbazole inhibitor (CheckPoint Therapeutics); E7016 (Eisai), PJ34 (2-(dimethylamino)-N-(6-oxo-5H-phenanthridin-2-yl)acetamide;hydrochloride) and 3-aminobenzamide.

Said platinum based therapy comprises platinum compounds such as cisplatin (Bristol Myers Squibb), carboplatin (Bristol Myers Squibb), oxaliplatin (Pfizer) and satraplatin (Yakult Honsha).

A taxane may be selected from cabazitaxel (Sanofi), docetaxel (Sanofi), paclitaxel (Celgene) and tesetaxel (Odonate Therapeutics). Said taxane preferably is paclitaxel, docetaxel or cabazitaxel.

Analysis of Patient Data that Identified Response Predictor Subtypes

This section describes the analysis of I-SPY2 patient data to generate the response predictor subtypes detailed above. Similar analyses can be performed on an expanded breaset cancer patient population and/or an alternative breast cancer patient population that includes therapeutic agents/treatment protocols not used in the analysis below to identify further response predictor subtypes.

The I-SPY2-990 mRNA/RPPA Data Resource: Patients and Data

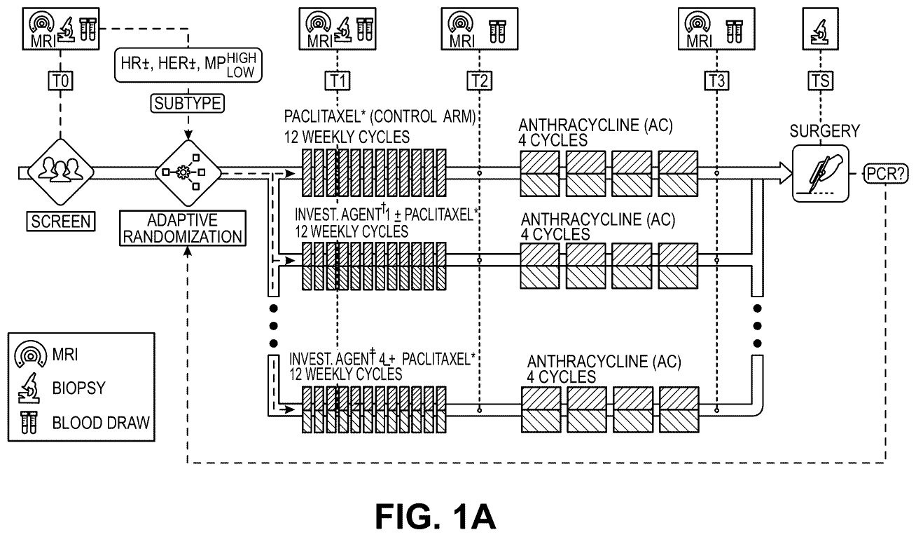

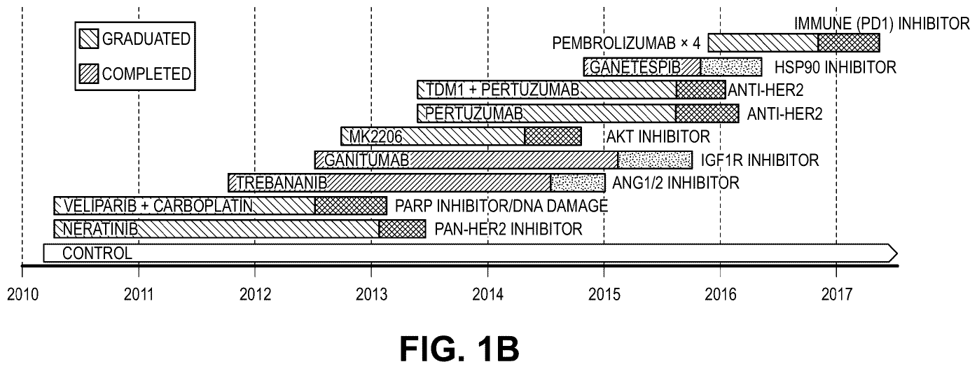

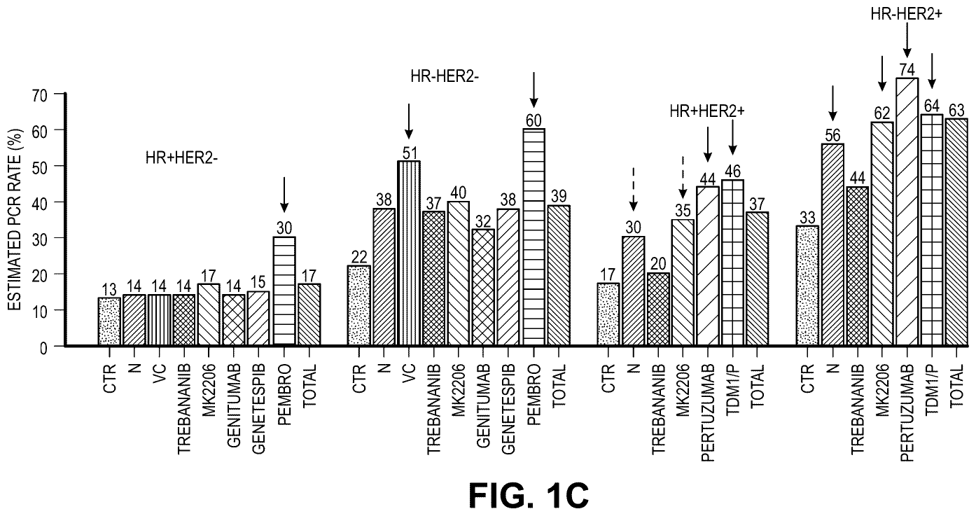

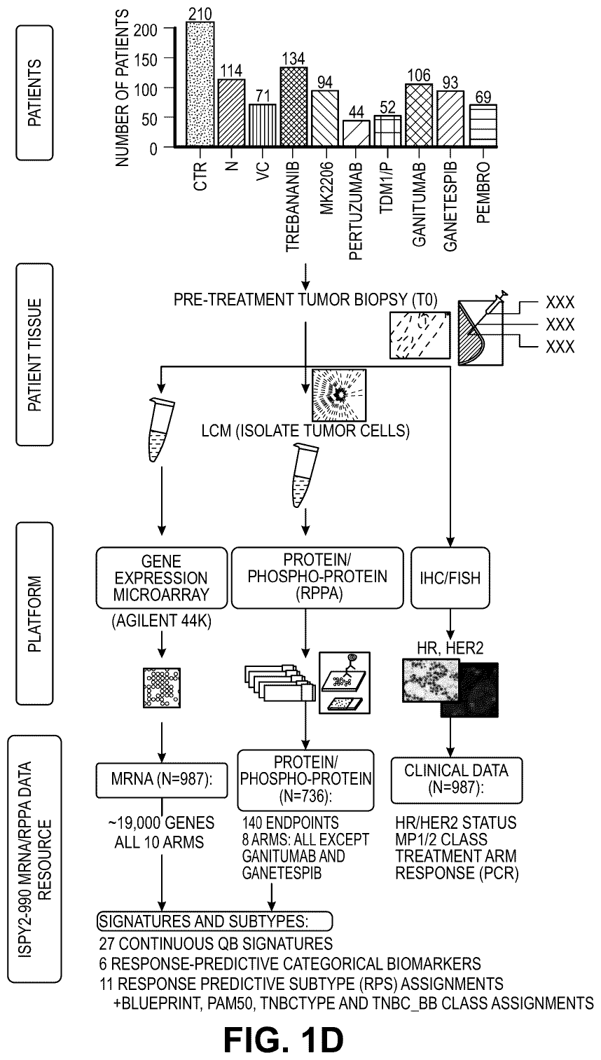

987 patients from 10 arms of I-SPY2 [210 Control (Ctr); 71 veliparib/carboplatin (VC); 114 neratinib (N); 93 MK2206; 106 ganitumab; 93 ganetespib; 134 trebananib; 52 TDM1/pertuzumab(P); 44 pertuzumab; 69 pembrolizumab (pembro)] were included in this analysis (FIGS. 1A and 1B). 38% of tumors were HR+HER2−, 37% triple negative (TN), and 25% HER2+(9% HR− and 16% HR+). Overall, 49% were classified MP (ultra) High-risk 2 (MP2) class, and 51% MP High 1 (MP1). 6 of these arms graduated within one or more receptor subtypes (purple bars) and 3 reached maximum accrual without graduation.

Estimated pCR rates by HR/HER2 receptor subtype for the 10 arms of the trial considered herein were previously reported and are summarized in FIG. 1C (Chien et al., 2019; Clark et al., 2021; Nanda et al., 2020; Park et al., 2016; Pusztai et al., 2021; Rugo et al., 2016). Even in the highest-efficacy treatment arms, 70% of HR+HER2−, 40% of triple negative (TN), 54% of HR+HER2+, and 26% of HR-HER2+ patients did not achieve pCR, further motivating the need for better biomarkers and subtyping schemas.

The I-SPY-990 data resource contains gene expression, protein/phosphoprotein and clinical data for the patients included in this analysis (FIG. 1D). All patients have pretreatment full transcriptome expression data on over ˜19,000 genes assayed on Agilent 44K. 736 patients (all arms except ganitumab and ganetespib have normalized LCM-RPPA data for 139 key signaling proteins/phosphoproteins in cancer (See Methods). Clinical data includes HR, HER2 and MP status, response (pCR or no pCR), and treatment arm. The ISPY2-990 Data Resource is publicly available in NCBI's Gene Expression Omnibus (GEO) ([GEO ID-record in progress]) and through the I-SPY2 Google Cloud repository (available at http www site ispytrials.org/results/data).

Predictive I-SPY2 ‘Qualifying’ Biomarkers Across 10 Arms of I-SPY2

Twenty-seven mechanism-of-action based gene expression signatures and proteins/phosphoproteins constituting our successful qualifying biomarkers reflect DNA repair deficiency (n=2), immune activation (n=8), estrogen receptor (ER) signaling (n=2), HER2 signaling (n=4), proliferation (n=3), (phospho) activation of AKT and mTOR (n=3), and ANG/TIE2 (n=1) pathways, among others (Table 1). Each pre-specified qualifying biomarker was originally found to predict response in a specific arm in one or more standard receptor subtypes, as previously reported (Lee et al., 2018; Wolf et al., 2018, 2017, 2020b, 2020a; Wulfkuhle et al., 2018; Yau et al., 2019). Table 1 also describes a newly developed VC-response biomarker for the TN subset (VCpred_TN) reflecting both DNA repair deficiency and Immune activation that was validated in BrighTNess (Loibl et al., 2018) and achieved qualifying status. In this analysis, we assessed whether they also predict response to different drugs included in other arms, with the goal of gaining biologic insight into which patients responded to what treatment and by what mechanism.

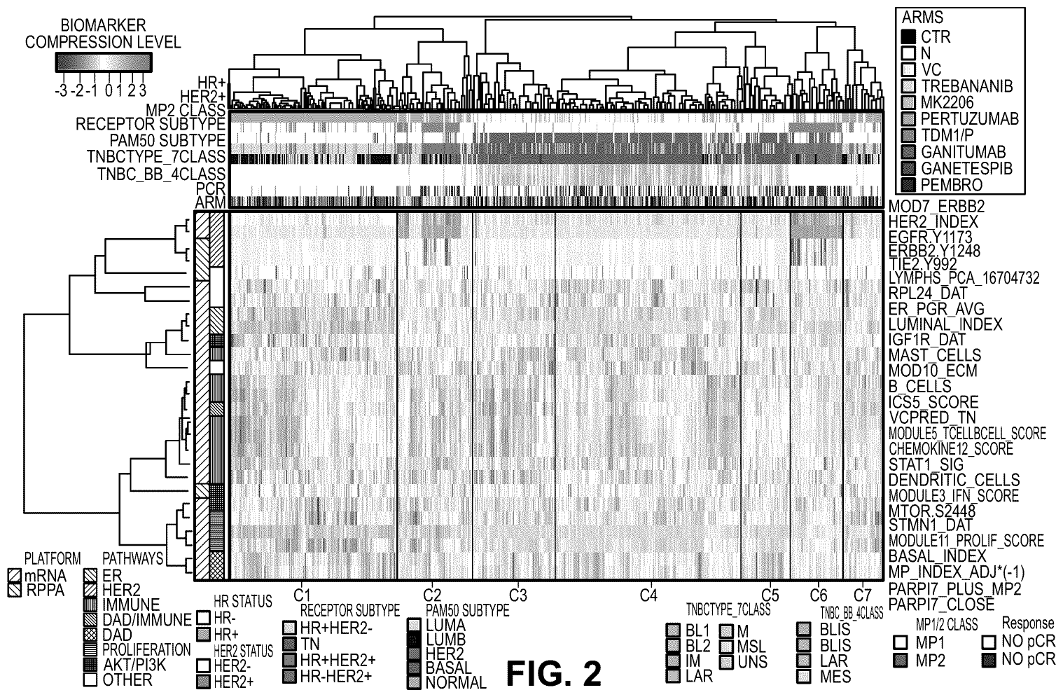

FIG. 2 shows the unsupervised clustered heatmap of qualifying biomarker expression levels. Biomarkers correlate by biologic pathway (FIG. 2, side dendrogram). Although patient profiles largely cluster by receptor subtype (FIG. 2), there is mixing between groups, highlighting the fact that for these patients, biological pathways other than HR/HER2 signaling are a stronger common denominator. Moreover, HR/HER2 sub-clusters appear to be characterized by immune-high (FIG. 2; C4, C6, C7, top dendrogram) and immune-low (FIG. 2; C1-3 and C5) signaling, though immune-high proportions differ by subtype (TN: 58%; HER2+: 41%; and HR+HER2−: 19%). Variability in ER/PGR, proliferation, and ECM signatures is visible as well.

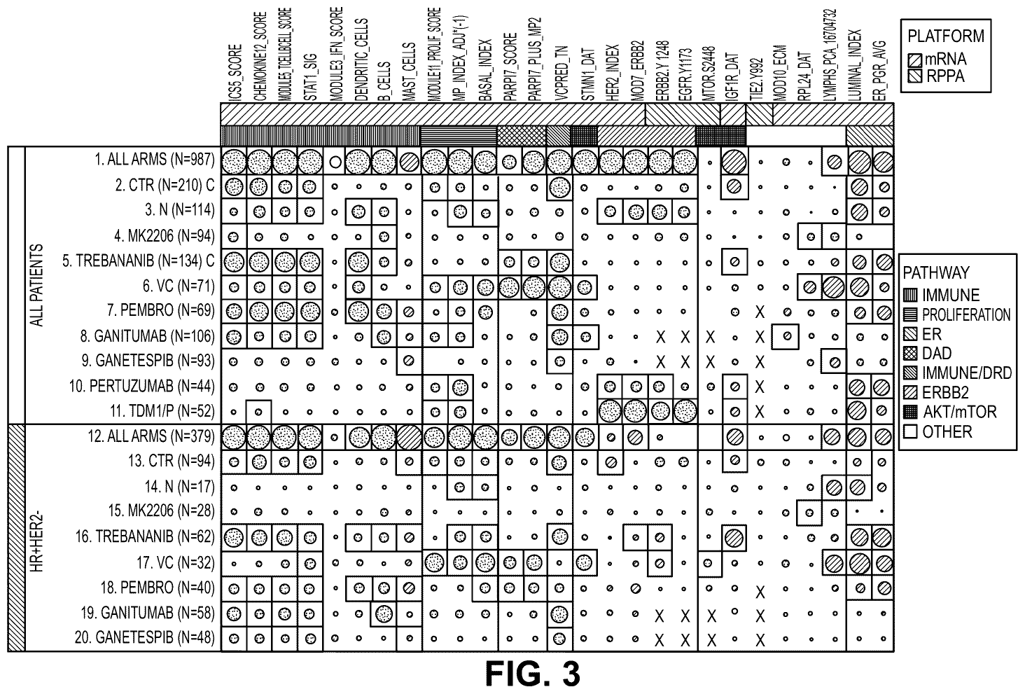

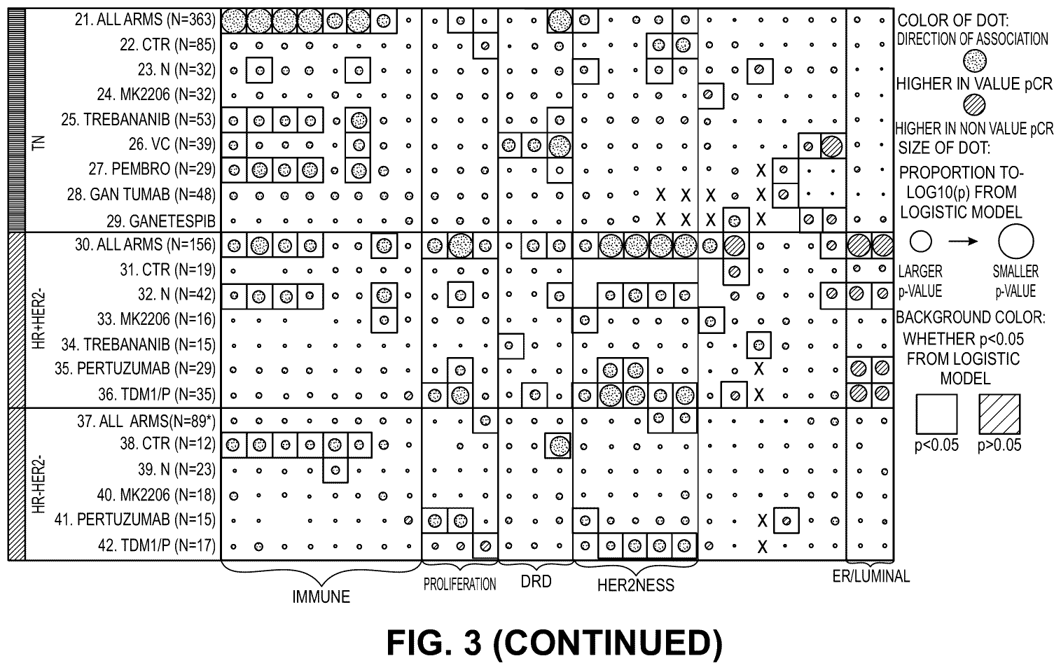

We used logistic regression to test the association of these 27 biomarker panels with pCR in all 10 arms individually, in the population as a whole (adjusting for HR, HER2 and treatment arm), and within receptor subtypes (FIG. 3 and Table 2). None of the 27 mechanism-of-action based biomarker panels associated with response exclusively in the arm where they were first proposed, indicating broader predictive function than anticipated.

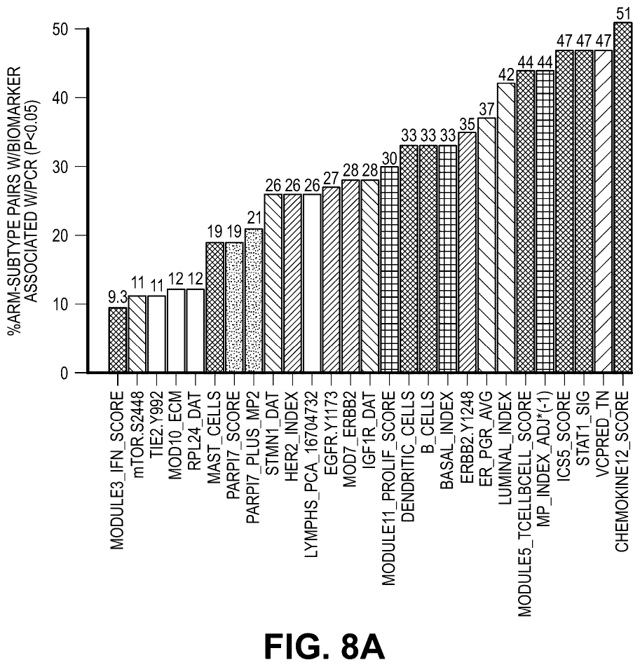

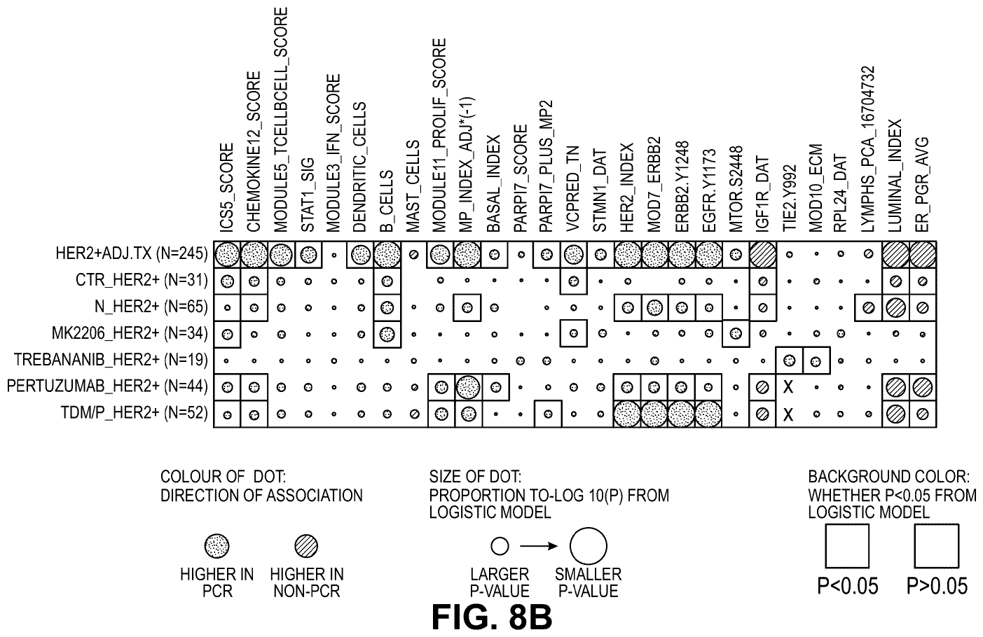

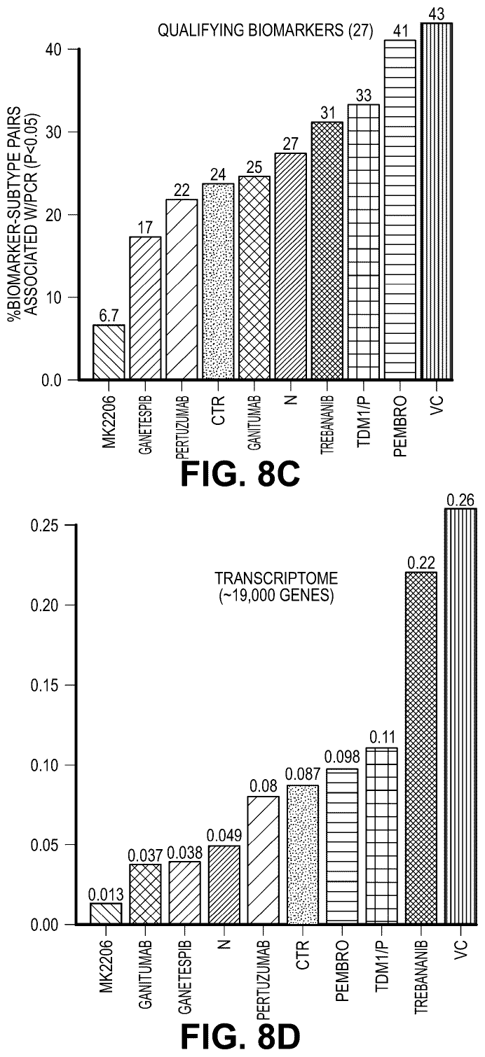

The biomarkers with broadest predictive function across drug classes were from immune, proliferation and ER/luminal pathways (FIG. 3 and FIG. 8A). One or more immune signatures predicted response in 9 of the 10 arms in the overall population (FIG. 3; rows 1-11, leftmost biomarker group-immune). However, different immune biomarkers were most predictive depending on receptor subtype and drug/drug class. For example, in the HER2+ subset, the B-cell gene signature predicts response to MK2206, neratinib and control chemotherapy, but is less predictive agents in the other arms (FIG. 3, rows 30-42; and FIG. 8B). In the TN subtype, the most predictive immune biomarkers are dendritic cells and STAT1_sig/chemokine12 gene signatures for pembrolizumab and the ANG1/2 inhibitor trebananib that affects macrophages and angiogenesis (FIG. 3; rows 21-29). All immune biomarkers were higher in pCR than non-pCR cases. The exception to the rule was the mast cell signature, which was higher in cases with residual disease (RD) in the HR+HER2− subtype, mainly due to its negative association with pCR in the pembrolizumab arm.

Proliferation biomarkers (i.e., adjusted MP index and basal index (continuous scores), and module11 proliferation score) were also broadly predictive of higher pCR overall (in 7 of 10 arms; FIG. 3—rows 1-11, second biomarker group from left-proliferation) and also in HR+HER2− (5/8 arms) and HR+HER2+(3/6 arms) subtypes (FIG. 3; rows 12-20 and 30-36), but generally not in TN or HR-HER2+ cancers (FIG. 3; rows 21-29 and 37-42).

Luminal/ER biomarkers (i.e. BluePrint_Luminal index, ER signature) predicted resistance to multiple therapies in the HR+HER2− subtype (5/8 arms: Pembro, Ctr, N, trebananib, and VC; FIG. 3, rows 12-20, rightmost biomarker group-‘ER/Luminal’). In HR+HER2+ and HER2+ subtypes they also associate with non-response in the HER2-only-targeted arms (control [trastuzumab+paclitaxel], N, THP and TDM1/P), but not in arms with agents that targeted other pathways (MK2206 or trebananib) added to trastuzumab (FIG. 3, rows 30-36; FIG. 8B). We also confirmed that HER2 biomarkers (i.e. HER2-EGFR co-activation, HER2index and Mod7_ERBB2 gene signatures) were predictive of pCR in multiple HER2-targeted arms (FIG. 3, fourth biomarker group from the left-‘HER2ness’). In the HR-HER2+ subtype, the BP-luminal and Her2ness did not generally predict response, other than Her2ness in TDM1/P (FIG. 3, rows 37-43).

In different HER2/HR subsets we also observe that the most specific biomarker (e.g., pMTOR for MK2206) may not be the most predictive (e.g. immune signals in the HER2+ subset in MK2206), and that phosphoproteins (e.g., pTIE2, pMTOR, pEGFR) may have greater predictive specificity than expression-based biomarkers (FIG. 3). Moreover, it appears that different biology may predict response to the same drugs in different receptor subtypes (e.g., trebananib: immune high in TN vs. pTIE2 in HER2+(FIG. 3 and (Wolf et al., 2018)); and MK2206: lower pMTOR in TN vs. higher pMTOR in HER2+(FIG. 3 and (Wolf et al., 2020a)). The number of significant biomarkers observed also differs by arm. Response to VC had the most significantly associated signatures and MK2206 the least (43% and 7% of biomarker-subtype pairs, respectively FIG. 8C). To assess whether this difference in the number of predictive biomarkers observed between agents is specific to the qualifying biomarker set selected, we performed whole-genome (n=19,000+ genes) analysis and observed similar results (FIG. 8D).

A Framework for Identifying a Response-Predictive Subtyping Schema for Prioritizing Therapies

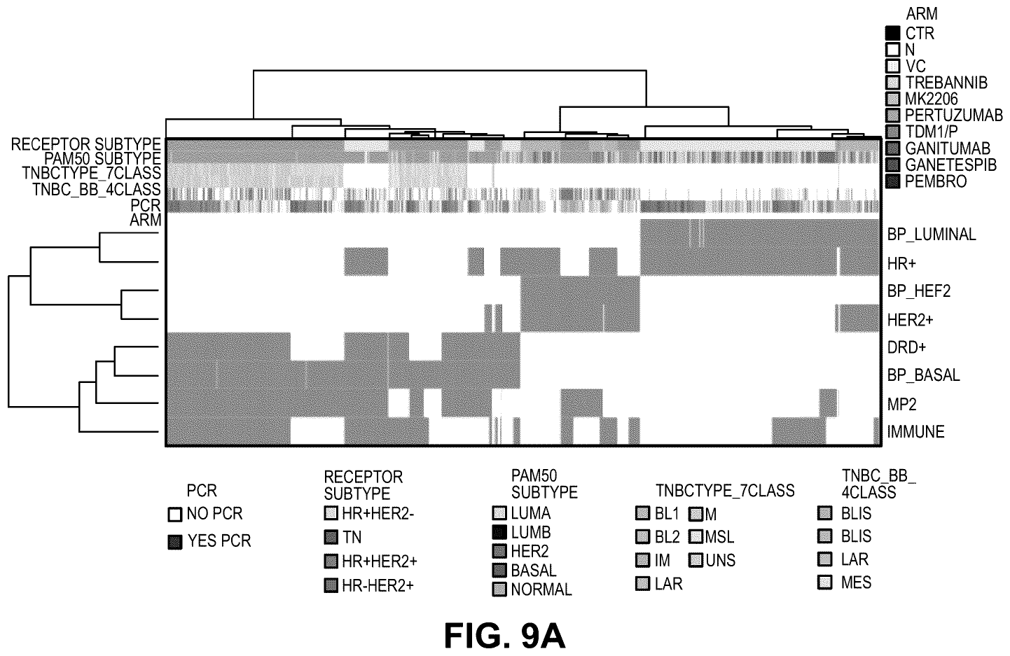

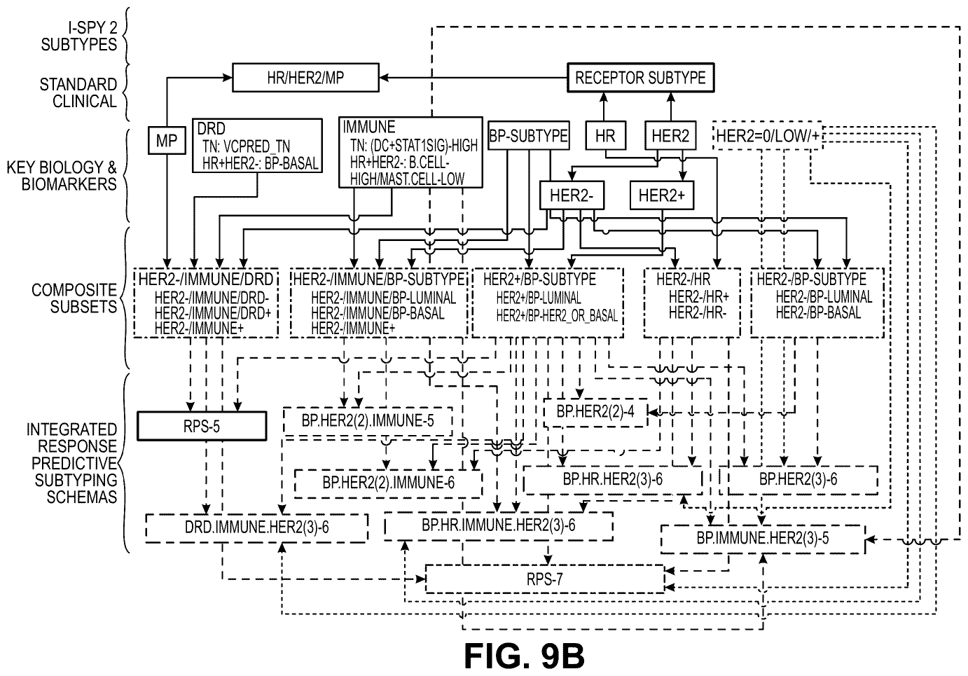

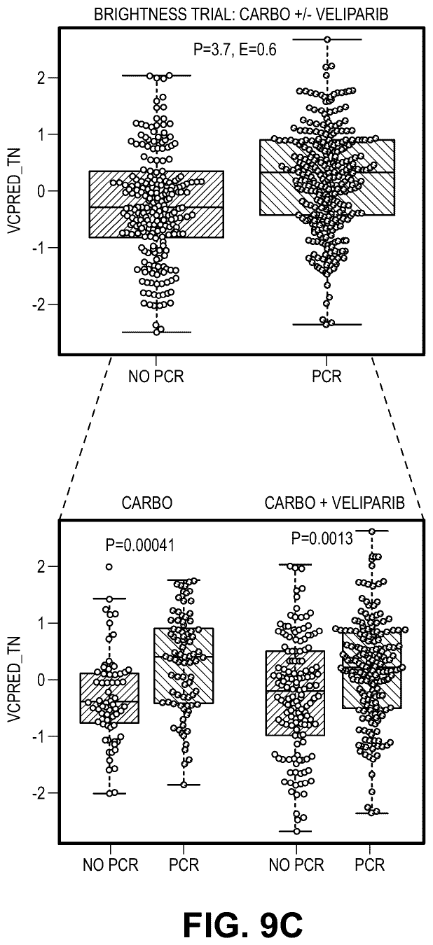

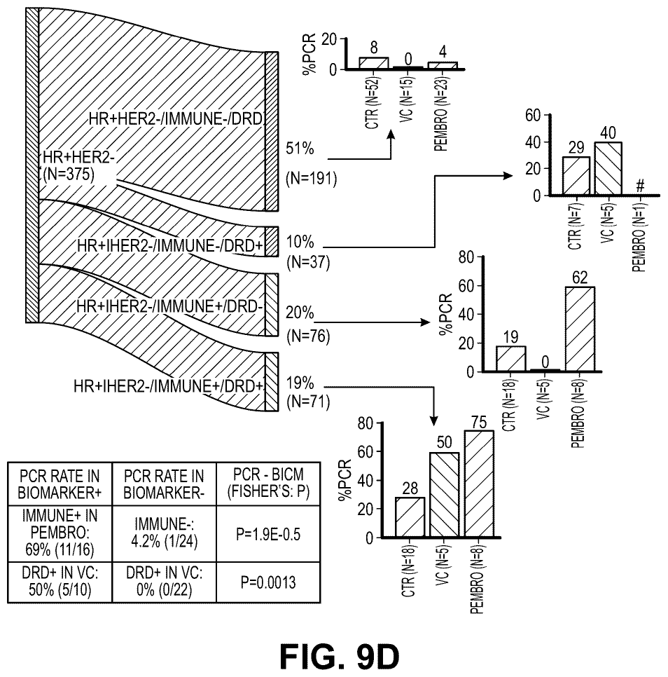

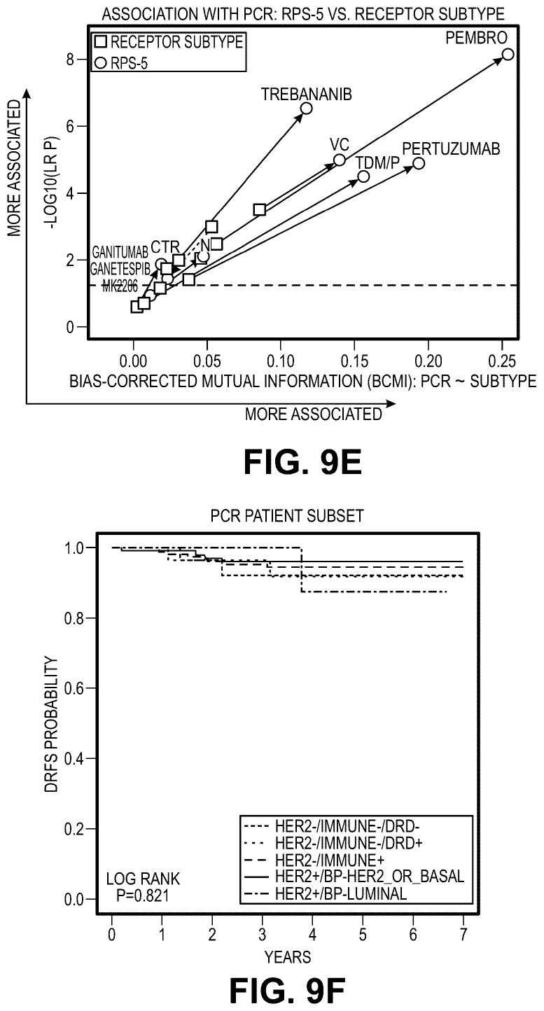

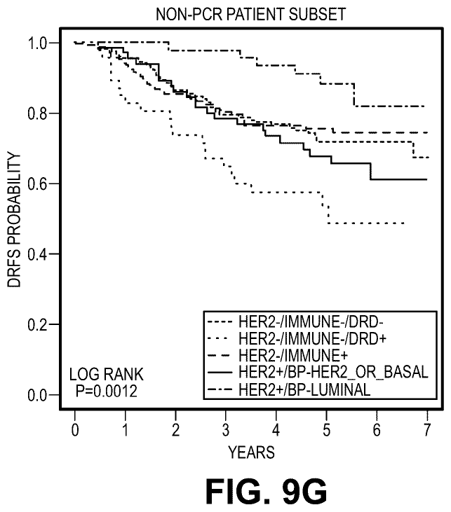

It is clear from our qualifying biomarker evaluation that within each HR/HER2 subtype, there is additional biology that further predicts response to I-SPY2 agents (FIG. 3). Candidate biological phenotypes that may add value to HR/HER2 include proliferation, DRD, Immune, luminal, basal, and HER2nes (FIG. 9A). Of the 11+ response-predictive subtyping schemas that we explored (FIG. 9B), our preferred schema incorporates biology that discriminates response to the treatments likely to be available in the clinic, such as platinum/PARP-inhibition and/or immunotherapy for HER2− patients, and dual-HER2 inhibition for HER2+ patients.

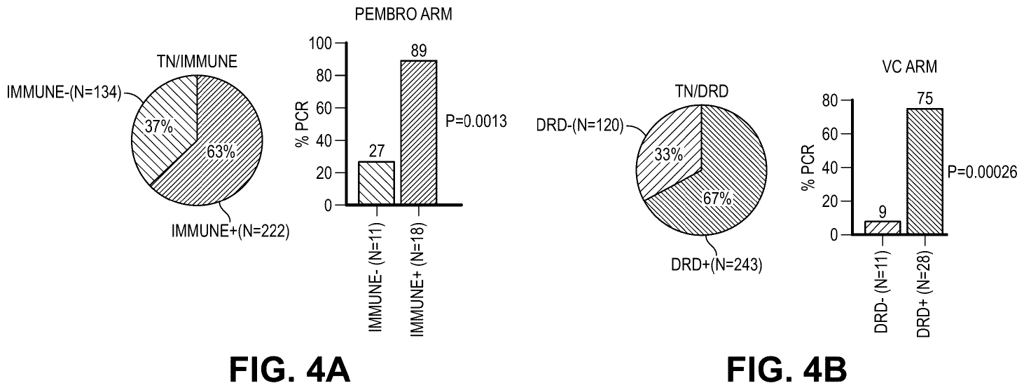

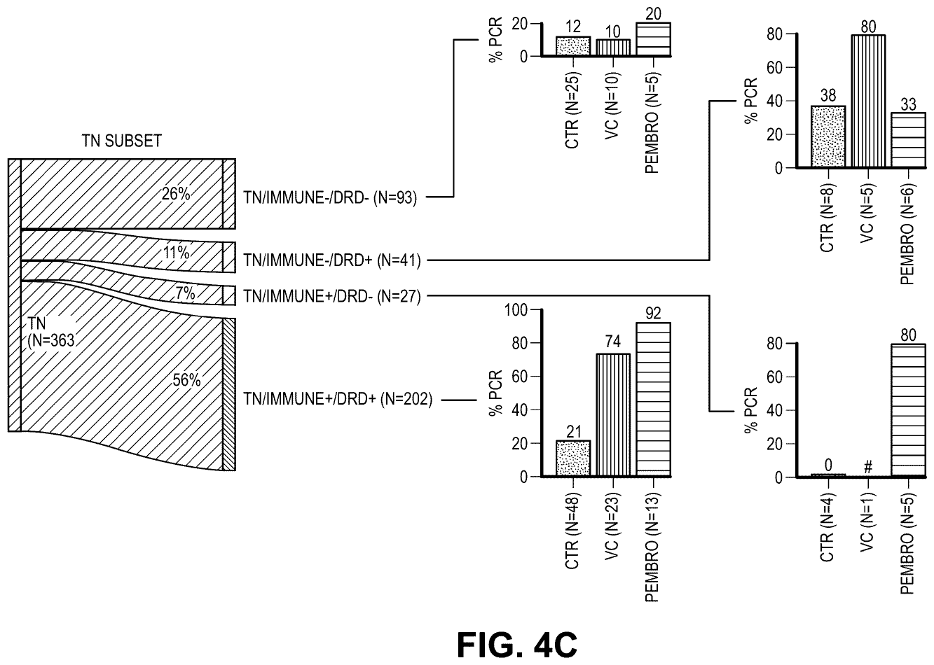

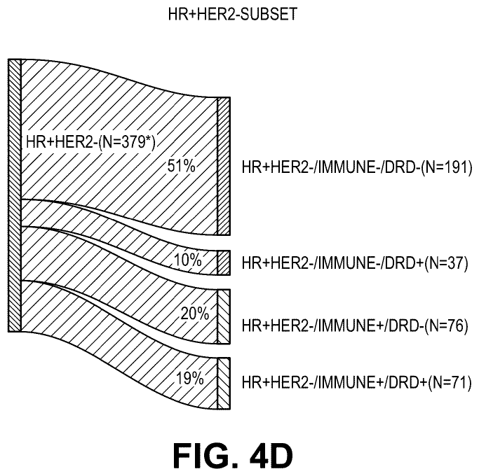

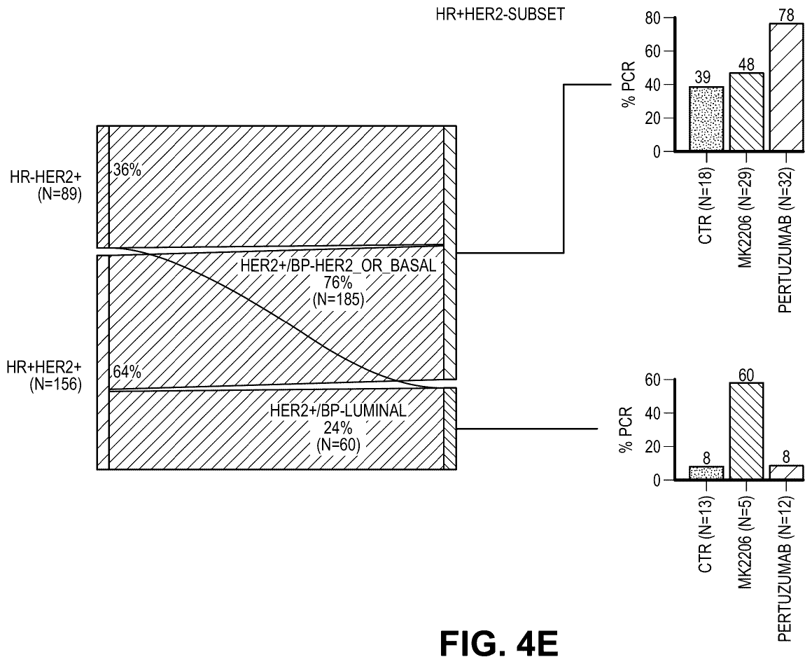

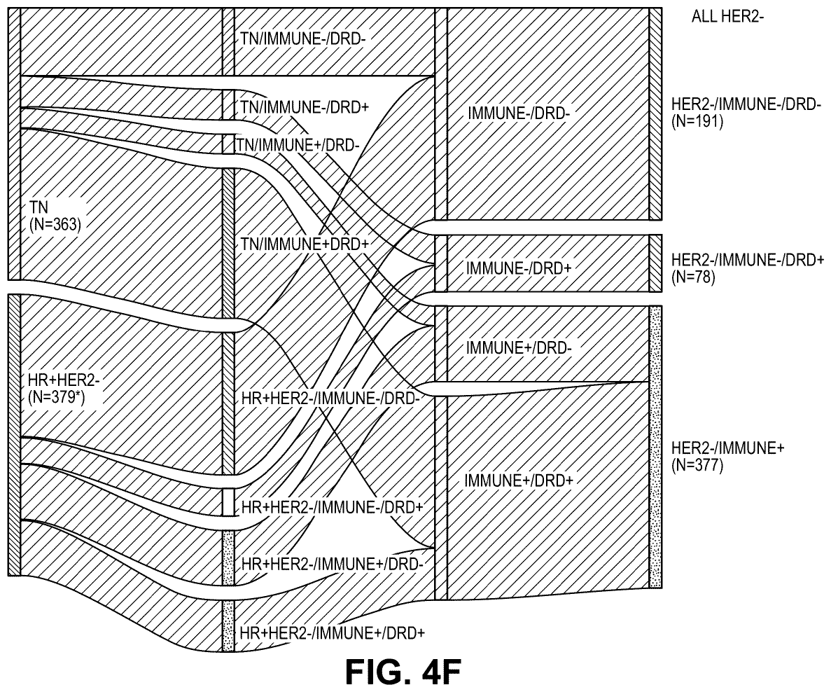

Our stepwise approach to developing this schema was as follows: Since platinum-based and immunotherapy—separately and together—are becoming the standard of care for TN breast cancer, we first examined the overlap between DRD/platinum-response and immune biomarkers as the putative drug class-specific predictors and calculated response rates to VC and Pembro in TN patients positive for one, both, or neither biomarker (FIG. 4A-4C; see Methods for biomarker implementation strategy). In TN, 67% were classified as DRD+, and 63% as Immune+(FIGS. 4A, 4B). Immune+TN patients had a high pCR rate to pembrolizumab (89%; FIG. 4A) and the DRD+TN patients had a high pCR rate to VC (75%; FIG. 4B). There is considerable overlap between Immune and DRD biomarker status in this subset of patients: 56% of TN are high for both biomarkers, 7% are Immune+/DRD−, 110% Immune−/DRD+, and 26% are Immune−/DRD− (FIG. 4C). The Immune+/DRD+ class had a very high pCR rate with either VC or pembrolizumab (pCR rates: VC: 74%, Pembro: 92%, control chemotherapy: 21%; FIG. 4C, bottom right). In contrast, the Immune+/DRD− class, had the highest pCR rate to pembrolizumab (Pembro: 80%; FIG. 4C, third down-right), whereas the Immune−/DRD+ class had the highest pCR to VC (VC: 80%, Pembro: 33%, control 38%; FIG. 4C, second down-right). For the 26% of Immune−/DRD− TN patients, response rates were very low in all arms (<21%; FIG. 4C, top right).

Given that Pembro graduated in I-SPY2 for efficacy in HR+HER2− and that a DRD+ subset was found responsive to VC (Wolf et al., 2017), we applied the same strategy for HR+HER2− cancers as for TN and examined the overlap between DRD and Immune status. Nineteen percent of HR+HER2− are positive for both biomarkers, 20% are Immune+/DRD−, 10% Immune−/DRD+, and 51% are Immune−/DRD− (FIG. 4D). While these proportions differ from those observed in TN, the pCR rates pattern is similar (FIG. 9D). We note here that our example implementation of these response-predictive phenotypes is subtype specific (e.g. Dendritic-cell and STAT1/chemokine signatures define Immune+ in TN whereas B-cell and Mast-cell signatures define Immune+ in HR+HER2−; see Methods.

In HER2+ cancers, motivated by the observation that high expression of the BP-luminal index or an ER related gene signature associated with lack of pCR in the HER2-only-targeted arms (i.e., control [trastuzumab], N, THP and TDM1/P), but not in arms targeting an additional pathway (i.e., MK2206 or trebananib) (FIG. 3), we defined a HER2+/Luminal phenotype and used the BluePrint subtypes to reclassify HER2+ patients by luminal signaling (FIG. 4E). The HR+HER2+, triple positive, patients were assigned almost evenly into HER2+/BP-Luminal+ and HER2+/BP-HER2_or Basal classes, whereas nearly all HR-HER2+ cancers were HER2+/BP-HER2 or Basal, and hardly any BP-luminal. For HER2+/BP-HER2 or BP-Basal patients, the pCR rate in the pertuzumab arm is 78%, versus 48% in the MK2206 arm, and 39% in control. In the HER2+/BP-Luminal class, 60% of patients achieved pCR in the MK2206 arm versus 8% in the pertuzumab and control arms, although very few patients received MK2206 and this finding requires further validation. Synthesis into a minimal set of response predictive subtypes: the RPS-5

Here we combine the predictive biology described above to include all patients in one classification schema. If we add Immune, DRD, and BP-Luminal/Her2 biomarkers to standard TN (FIG. 4C), HR+/HER2− (FIG. 4D), and HER2+(FIG. 4E) status per above, a 10-subtype schema would result. With 10 subtypes, some would include only a handful of patients and be difficult to statistically evaluate in a trial setting. Given this practical consideration, we combined all Immune+ patients in HR+HER2− and TN subsets into a single subtype HER2−/Immune+(FIG. 4F, right-bottom), as both subsets share pembrolizumab as the same best (highest pCR) agent (see FIG. 4C and FIG. 9D). We also combined TN/Immune−/DRD+ and HR+HER2−/Immune−/DRD+ patients into the subtype HER2−/Immune−/DRD+(FIG. 4F, right-middle), as these subsets share VC as the highest-pCR arm (see FIG. 4C and FIG. 9D). With this schema, we can create the 5 novel subtypes that define the RPS-5 response-predictive subtyping schema (combined FIG. 4F and FIG. 4E, respectively): HER2−/Immune−/DRD−, HER2−/Immune−/DRD+, HER2−/Immune+, HER2+/BP-HER2-r Basal, and HER2+/BP-Luminal.

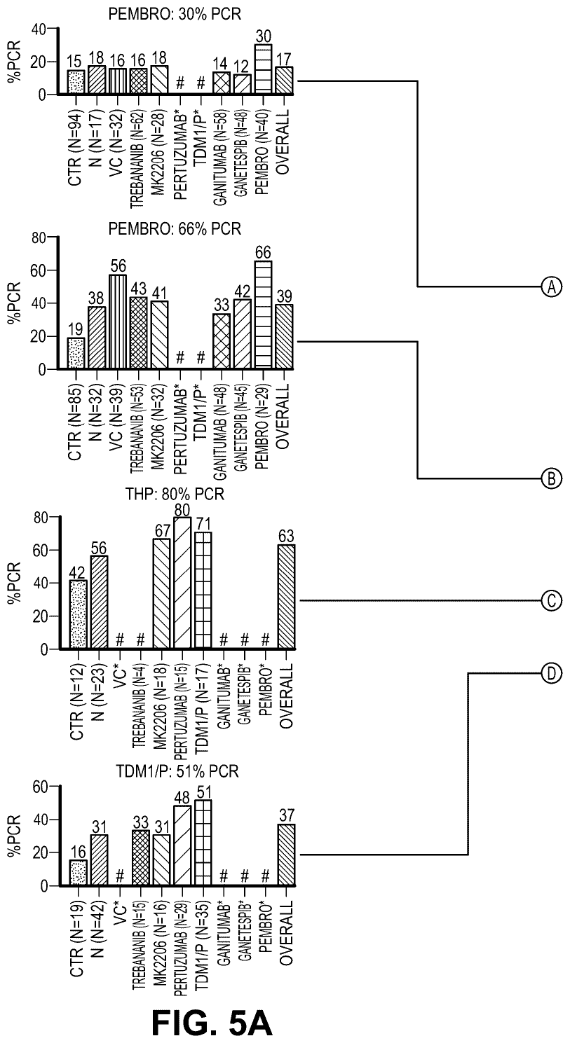

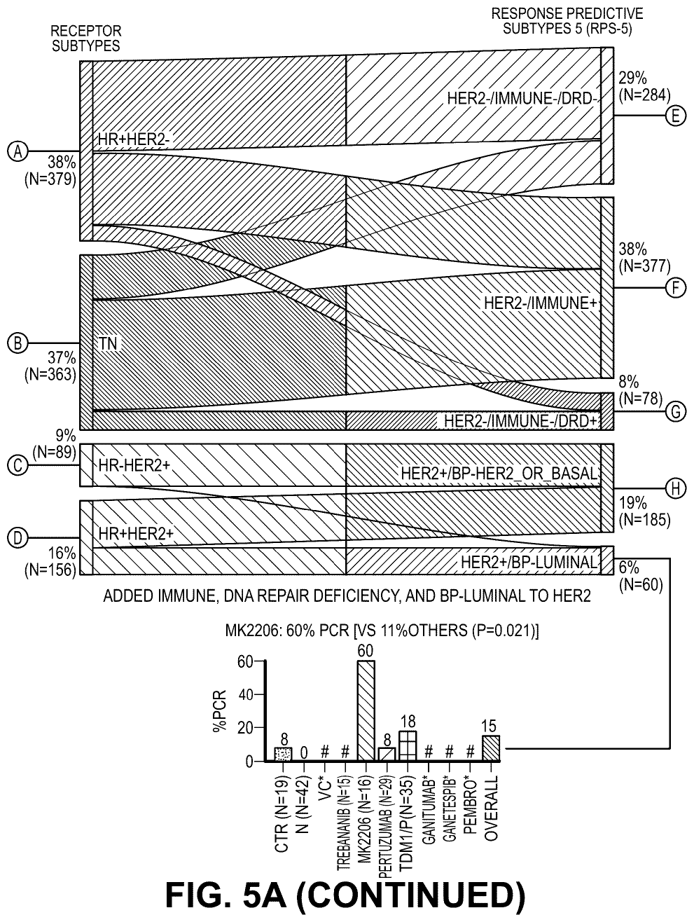

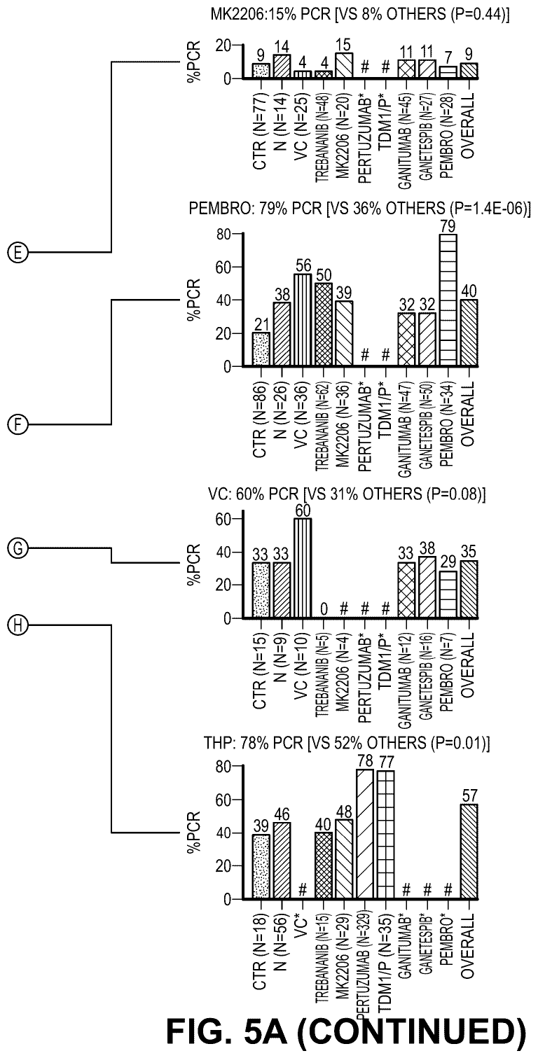

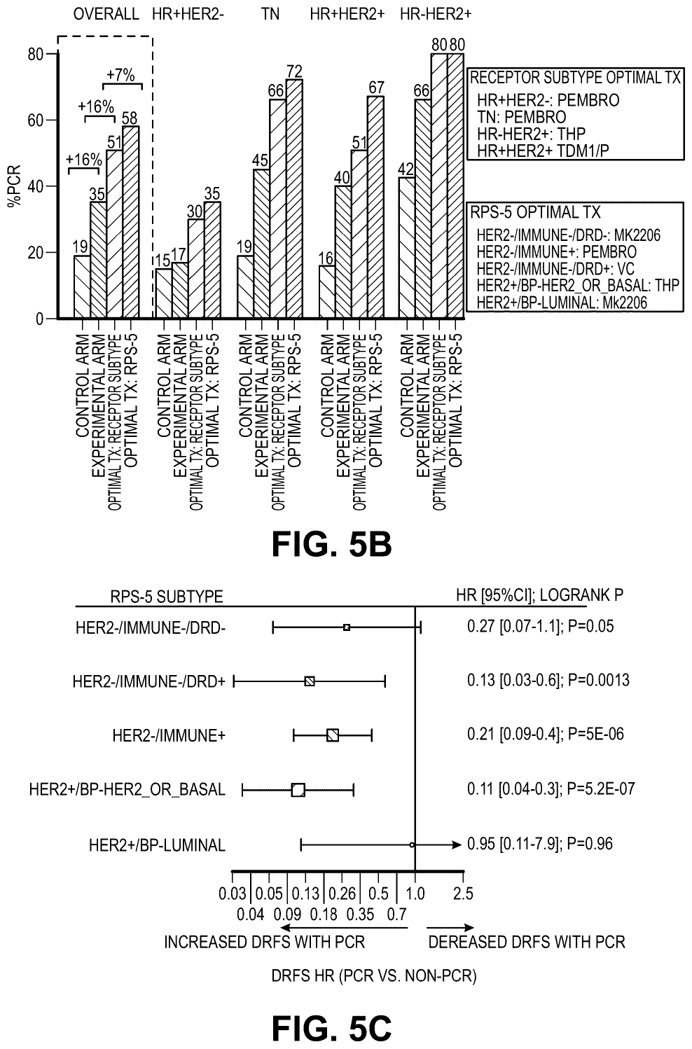

The Sankey diagram in FIG. 5A shows the relationship between standard receptor subtypes and the new RPS-5 subtyping schema in the I-SPY2 data. Receptor subtypes and their prevalence are shown on the left (starting with 38% HR+HER2−, 37% TN, 16% HR+HER2+, and 9% HR-HER2+) and the plot illustrates how receptor subtypes ‘flow’ into the new RPS-5 subtypes on the right (stratifying into 29% HER2−/Immune−/DRD−, 38% HER2−/Immune+, 8% HER2−/Immune−/DRD+, 19% HER2+/BP-HER2 or Basal, and 6% HER2+/BP-Luminal). pCR rates by drug arm within each subtype are shown in the barplots to the left for the standard receptor subtypes and to the right for the new RPS-5 subtypes.

Using the standard HR/HER2 receptor subtype to classify patients reveals that arms with the highest pCR rates include pembrolizumab for HR+HER2− and TN cancers with 30% and 66% pCR rates, respectively; pertuzumab for HR-HER2+ cancers with 80% pCR and TDM1/P for the HR+HER2+ subtype with 51% pCR. Using the RPS-5, the best drugs are pembrolizumab for HER2−/Immune+ with 79% pCR; VC for the HER2−/Immune-DRD+ cancers with 60% pCR; and MK2206 for HER2−/Immune−/DRD− cancers with 20% pCR though all arms performed similarly with low pCR in this subtype. In the HER2+ cancers, the best drug was pertuzumab for HER2+/BP-HER2_or_Basal cancers with 78% pCR; and MK2206 for HER2+/BP-Luminal cancers with 60% pCR, though numbers are small.

Impact of Classification Schema on Trial Population Level pCR Rates and Maximization of Patient Benefit

A major goal of a response-predictive subtype schema is to increase the pCR rate in the population and to maximize the probability of pCR for an individual patient. To examine the impact of the new RPS-5 schema, we performed an in silico experiment to calculate how the overall pCR rate would compare if treatments in the multi-arm adaptive randomization I-SPY2 trial (FIG. 1A) had been assigned according to the RPS-5. The observed overall pCR rate in the standard of care control arm of I-SPY2 was 19% (black bar, FIG. 5B, under “Overall”). In the 9 experimental arms of the trial taken together, the actual observed overall pCR rate was 35%, a 16% increase over the control arm (orange bar, FIG. 5B). Had patients been assigned to the best experimental treatment arm (that became apparent only in hindsight) based on standard receptor subtypes, the estimated overall pCR rate in the experimental arms all together would have been 51%, a further 16% increase (red bar, FIG. 5B). Finally, if we had assigned patients using the new RPS-5 to their corresponding best treatment, the overall pCR rate in the combined experimental arms would be 58%, a further 7% improvement (blue bar, FIG. 5B). Achieving a pCR results in excellent patient outcomes in all RPS-5 subtypes (FIG. 9E, 9F). However, similar to differences observed among HR/HER2 subtypes, the relative survival benefit varies from RPS-5 subtype to subtype as well, with the highest hazard ratios observed in HER2−/Immune−/DRD+, HER2−/Immune+, and HER2+/BP-HER2_or_Basal (FIG. 5C, FIG. 9G).

The gain in pCR rate from RPS-5 reclassification is not evenly distributed across HR/HER2 subtypes. As illustrated to the right in FIG. 5B, in the HR-HER2+ subtype there is no pCR increase by switching to the RPS-5 as they are all within the HER2+/HER2-or-basal subtype, whereas in the HR+HER2+ receptor subtype switching to the RPS-5 could increase pCR rate by 16% (from 51% to 67%). In addition to boosting response rates over the population, a good subtyping schema should also discriminate between responders and non-responders over a wide range of treatment classes. We use bias-corrected mutual information, which quantifies the amount of uncertainty about pCR probability that is reduced by knowing subtype versus not knowing it, to compare the predictive power of different subtyping schemas. To visualize the pCR-predictive goodness of the RPS-5 schema vs. receptor subtype we plot association p-value vs. bias-corrected mutual information for both classification schemas in each arm of the trial (FIG. 9E). For most drug arms (7/10), the RPS-5 schema is more predictive of pCR than receptor subtype as can be seen by the higher concentration of points in the upper right quadrant with high BCMI and low p-values (FIG. 9E).

Adapting Response-Predictive Subtyping Schemas to a Rapidly Evolving Treatment Landscape

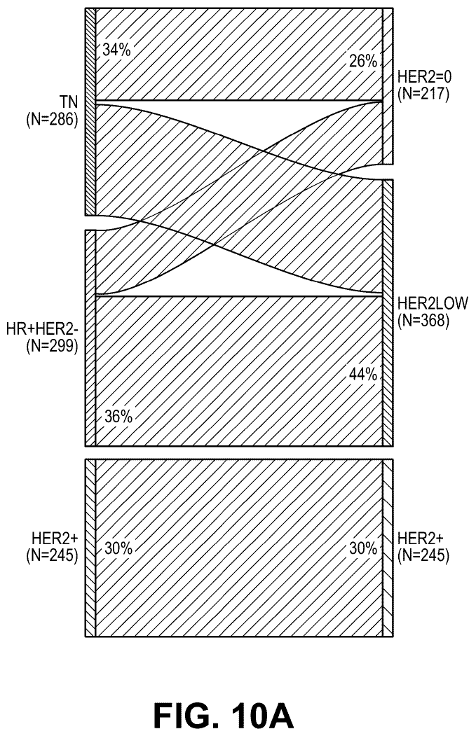

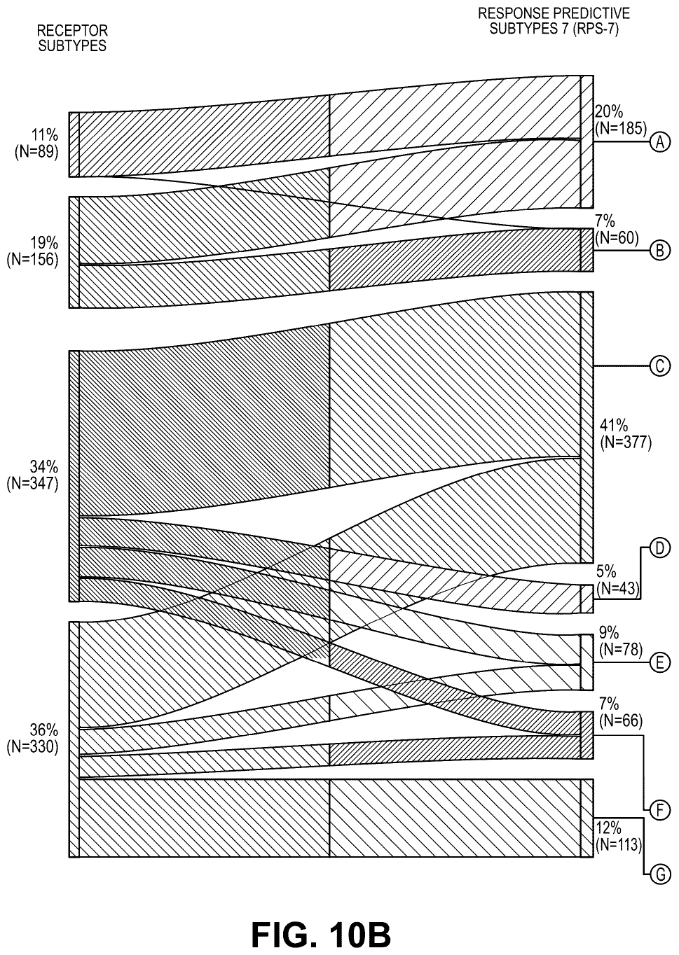

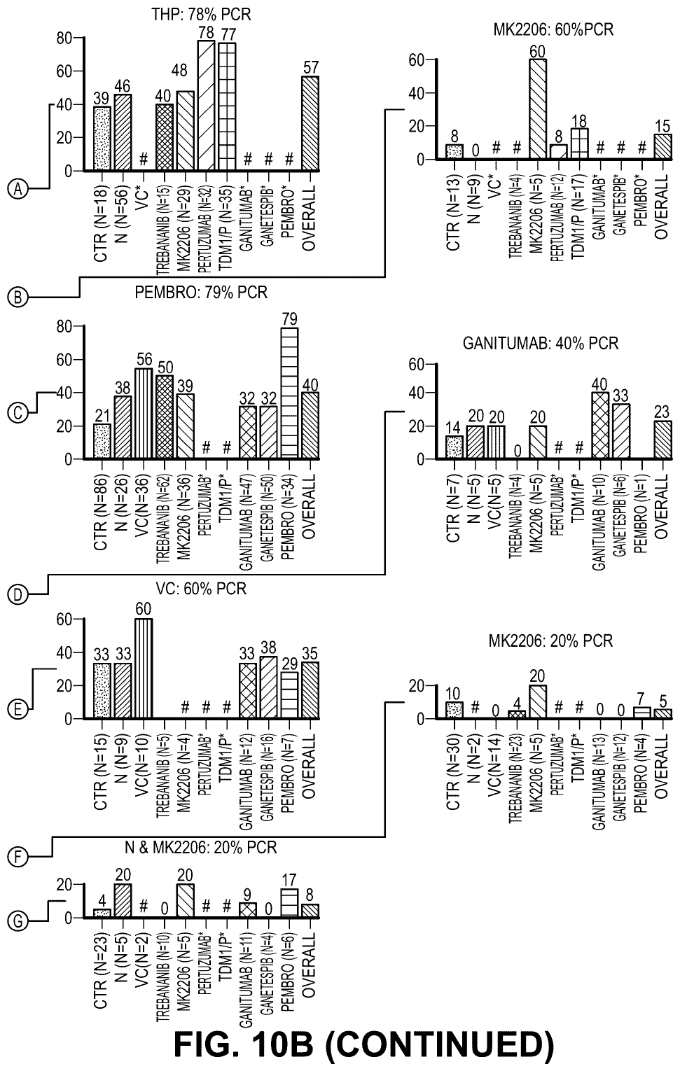

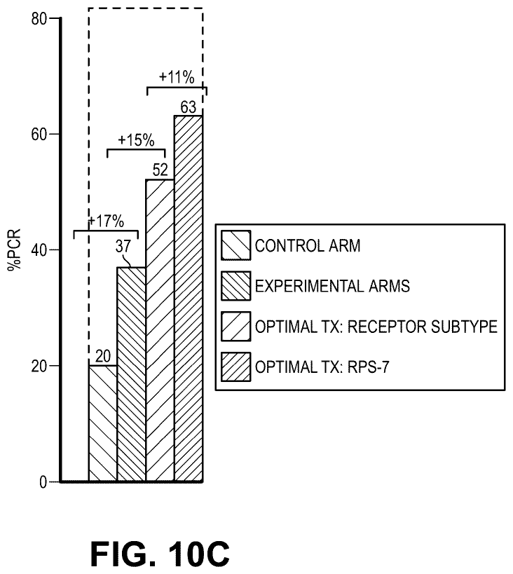

Adding new drug classes to the trial in the future may call for incorporation of new biomarkers and necessitate revisions to the classification schema. For example, an agent targeting HER2-low cancers, defined as HER2 IHC 2+ or 1+ and FISH-negative, is currently being evaluated in I-SPY2. If we transform HER2 status from the binary HER2+/− classes to 3 levels (HER2=0, HER2low, and HER2+) as shown in the Sankey diagram in FIG. 10A, and integrate it with Immune, DRD, HR, HER2, and BP_Luminal, we arrive at a new 7-subtype schema, the RPS-7, with subtypes S1: HER2+/BP-HER2_or_Basal, S2:
HER2+/BP Luminal, S3: HER2=0.or.low/Immune+, S4: HR−/HER2low/Immune−/DRD−, S5: HER2=0.or.low/Immune−/DRD+, S6: HER2=0/Immune−/DRD−, and S7:
HR+HER2low/Immune-DRD− (FIG. 10B). Agents yielding the highest pCR rates are THP [78%], MK2206 [60%], Pembro [79%], ganitumab [40%], VC [60%], N or MK2206 [20%], and MK2206 [20%] for S1-7, respectively. This schema adds 11% pCR over optimal assignments using receptors only, even without a HER2 low targeted agent (pCR: 63% vs. 52%, FIG. 10C).

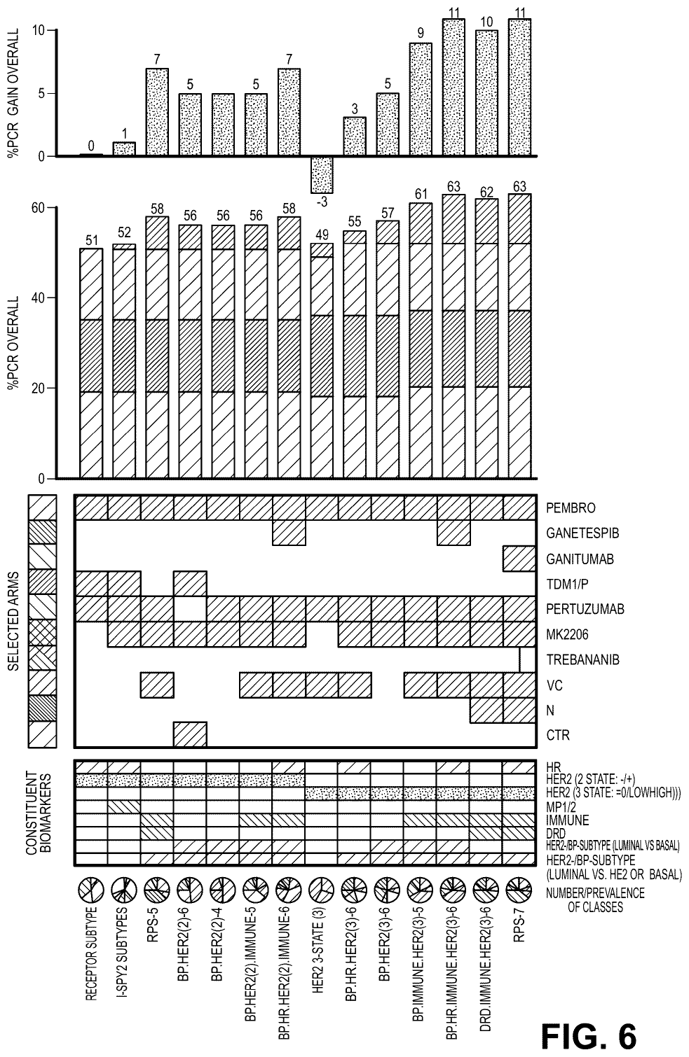

The characteristics and relative pCR rates of RPS-5, RPS-7, and the nine other subtyping schemas defined in FIG. 9B are summarized in FIG. 6. For example, the RPS-5 (third column from left) creates 5 classes defined by HER2, Immune, DRD, and Luminal status, that if used to prioritize treatment arms by class would select Pembro, Pertuzumab, MK2206, and VC and result in a pCR rate of 58% overall in the I-SPY2 population, a 7% gain over the maximum possible for receptor status. Similarly, the composition and performance of the RPS-7 (rightmost column) is summarized per above, including its selection of ganitumab and neratinib as the best agent within a subtype. Looking at these schemas together, we observe that different schemas select different ‘best’ treatments. Some agents are optimal for at least one subtype in nearly all schemas (e.g., Pembro and Pertuzumab), while some are not selected in any schemas. Some agents are only selected when biological phenotypes in addition to HR/HER2 are incorporated (e.g. MK2206). All agents that graduated for efficacy appear as optimal in at least one schema, and two—Ganetespib and Ganitumab—that did not graduate for efficacy were selected as optimal in schemas incorporating the classes TN/Immune−/Basal or TN/HER2low/Immune−/DRD−, including the RPS-7, an illustration that conventional HR/HER2 subtyping may not be able to identify a responding subset. Estimated maximum pCR rates differ by subtyping schema as well, ranging from 49% to 63%, suggesting a cap of <65% pCR for the 10 treatments included in the ISPY2-990, irrespective of biomarker-based treatment assignment schema.

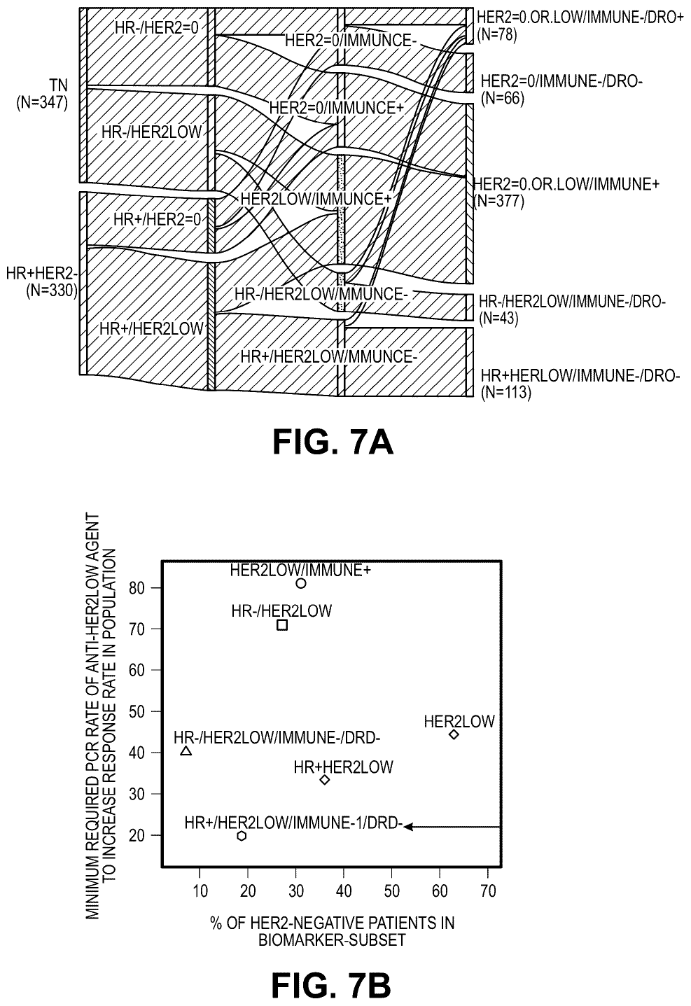

The RPS-7 and other HER2 3-state-containing schemas also illustrate that when introducing a new class of agent such as a HER2low inhibitor, the minimum required efficacy to improve pCR rates depends strongly on the biomarker-subset in which it is tested. For example, in RPS-7 HER2low patients fall into four groups (RPS-7 classes S3-S5 and S7), with pCR rates to the most efficacious agent ranging from 20% to 70% with current I-SPY2 therapies (FIG. 10B). In addition, other relevant HER2low subsets may include all HER2low or HR+HER2low, among others (FIG. 7A). If tested in the HR+/HER2low/Immune−/DRD− group, a HER2low agent only has to reach a pCR rate of 20% to exceed the maximum response currently attainable from any agent tested so far in the trial (FIG. 7B). This subset constitutes 20% of all HER2−, and 38% of HR+HER2− patients in the I-SPY2 trial. In contrast, if the developer were to test the agent in all HER2low patients, although the prevalence is higher (˜65% of HER2−), the minimum efficacy for adding value to the I-SPY2 agent arsenal is considerably higher at 44% pCR (FIG. 7B).

### SUMMARY

The I-SPY2-990 mRNA/RPPA Data Resource data compendium described herein contains containing pre-treatment gene expression data, tumor epithelium specific protein/phosphoprotein data and clinical/response information for ˜990 breast cancer patients from the first 10 completed arms of the I-SPY2 neoadjuvant chemo-/targeted-therapy platform trial for high-risk, early-stage breast cancer. These high quality molecular data from common protocols and a centralized workflow provide a valuable resource containing patient-level response data to a wide variety of anti-cancer agents with very different mechanisms of action, including DNA damaging agents (platinum, anthracycline), PARP inhibitors, AKT inhibitors, angiogenesis inhibitors (Ang1/2; Tie2), immunotherapy (PDT), small molecule pan-HER2 inhibitors, and dual-HER2 targeting therapies.

The data have been used to power our Qualifying (hypothesis testing) and Exploratory (discovery/hypothesis generating) Biomarker programs, where we have tested previously published mechanism-of-action biomarkers as predictors of response to platinum-based therapy (Wolf et al., 2017), neratinib (Wulfkuhle et al., 2018), AKT-inhibitor MK2206 (Wolf et al., 2020a), PD1 inhibitor pembrolizumab (Gonzalez-Ericsson et al., 2021), dual anti-HER2 therapies TDM1/P and Pertuzumab (Clark et al., 2021; Wolf et al., 2020b) and anti-Ang1/2 therapy trebananib (Wolf et al., 2018), among others (Kim et al., 2021). These examples extended our previous work by assessing the performance of successful biomarkers across arms and found that all examined biomarkers associated with response in at least one arm other than the one where they were proposed as predictors. Expression signatures from immune, proliferation and ER/luminal pathways are predictive of response to multiple regimens targeting diverse pathways in multiple subtypes, including HER2-targeted agents for HER2+ subtypes. In contrast, phosphoproteins from HER2, EGFR, AKT/mTOR and other pathways appear specific in predicting response to agents targeting related mechanisms of action. More generally, we found that the most specific biomarker may not be the most predictive, and that different receptor subtypes may have different predictive biomarkers to the same agents.

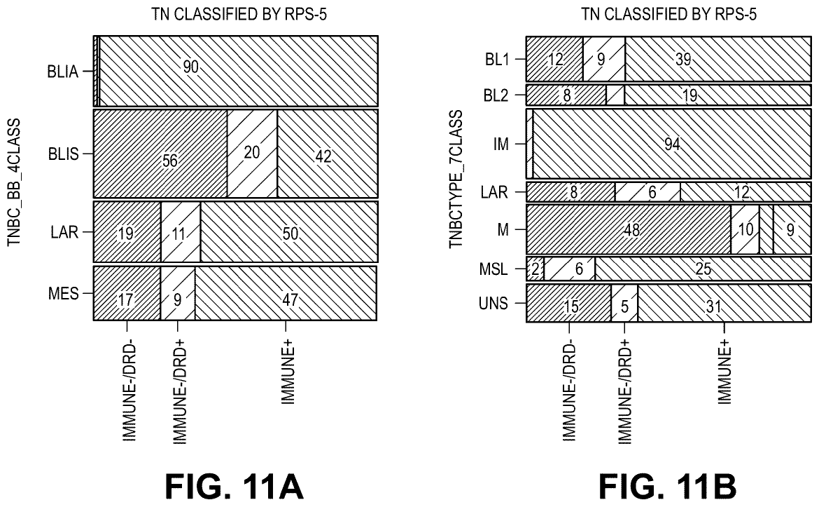

The biomarker results in this larger 10-arm context provide a more refined understanding of who responds to which therapy and why. Responders to immunotherapy have high levels of immune signatures, but different receptor subtypes seem to have different predictive biology: high dendritic, chemokine, and STAT1 cells/signals best predict response for TN, whereas high B-cell combined with low mast cell best predict pCR in HR+HER2−. Within the TN subset, these immune signals are high in the Brown & Burstein (Burstein et al., 2015) and Lehmann (Chen et al., 2012; Lehmann et al., 2011) immune-rich TN subtypes (FIG. 11), but many patients outside these (small) classes also have high levels of immune-predictive signatures, as reflected in the high prevalence of Immune+ patients in our example implementation. An exploratory cross-platform immune expression biomarker analysis further details immune subpopulations and their association with response (Yau et al., 2019). RPPA-based quantitative tumor epithelium MHCII levels and activation (phosphorylation) of STAT1 at pre-treatment were recently found to strongly associate with response to both pembrolizumab in I-SPY2 (Nanda et al., 2020) and durvalumab in the neo-adjuvant setting (NCT02489448)(Gonzalez-Ericsson et al., 2021). Platinum agent plus PARP inhibitor veliparib response is predicted by high DRD and STAT1-related immune signaling in TN and by both DRD and high proliferation in the HR+HER2-subset. HER2+ dual-HER2 targeted therapy responders tend to have higher HER2 signaling on expression, protein, phosphoprotein levels, with proliferation signals providing potential discrimination of response between TDM1/P and THP in the HR+HER2+ subset (Clark et al., 2021).

We then applied these insights and clinical considerations to develop novel response-predictive subtyping schemas that incorporate tumor biology beyond clinical HR/HER2 status that may better inform agent selection in a modem treatment landscape. Candidate ‘fit for purpose’ biological phenotypes to add to HR/HER2 included proliferation, DRD, Immune, luminal, basal, and HER2ness, selected because they predict response to newer agent classes likely to be found in the clinic today. However, when so many phenotypes are considered, there is a combinatorial explosion in the possible number of marker states, and many ways to collapse them into smaller useful response-predictive subtyping schemas. To help sort through the options, we reasoned that an ideal response-predictive subtyping schema should: 1) differentiate optimal treatments, meaning that different subtype classes should have different ‘best’ treatments yielding the highest pCR probability; 2) result in a higher pCR rate in the population if used to optimally assign/prioritize treatments; 3) differentiate between responders and non-responders over a wide range of treatments; and 4) be robust to platform and applicable across different drugs with the same mechanism of action and simple to implement clinically.

Of the 11+ potential mRNA expression-based response-predictive subtyping schemas we explored, we selected the treatment Response Predictive Subtype 5 (RPS-5) for prospective evaluation in I-SPY2. This schema was motivated by clinical considerations in TN and HER2+. Both immunotherapy and platinum-based therapy arms graduated in the TN subset in I-SPY2. These results were subsequently validated in the large randomized trials BrighTNess (Loibl et al., 2018) and KEYNOTE-522 (Schmid et al., 2020). These drugs are now increasingly used in clinical practice individually or together. We classified TN patients by Immune and DRD markers to determine whether the same, or different, populations are responding to each class of therapy and whether this information could be used to spare patients the toxicity of combined platinum-based and immunotherapy if both are not needed to achieve pCR. We applied the same stratification to HR+HER2− patients based on the efficacy of Pembro, the many immune markers associated with response in that arm and other immunotherapy arms in I-SPY2, and previous work showing that responders to VC can be identified by DRD biomarkers such as PARPi7 combined with MP2 class (Wolf et al., 2017), and also by the BluePrint(BP)-Basal subtype (Krijgsman et al., 2012). We used BP-Basal classification as our measure to assess the DRD phenotype in HR+HER2− because the assay is performed in a CLIA setting and is ready for clinical implementation with a pending IDE application submission to the US FDA, even though the research assay based PARPi7-high/MP2 performed somewhat better in this dataset. HER2+ patients were re-classified by luminal signaling to better identify subsets likely to respond to dual-anti-HER2 therapy vs. those that may need a different approach.

The resulting, simplified RPS-5 has five subtypes: HER2−/Immune−/DRD−, HER2−/Immune+, HER2−/Immune−/DRD+, HER2+/BP-HER2 or Basal, and HER2+/BP-Luminal. Using this schema to maximize pCR rates, one would prioritize platinum-based therapy for HER2−/Immune−/DRD+, checkpoint inhibitor therapy for HER2−/Immune+, and dual-anti-HER2 therapy for HER2+ that are not luminal. HER2+/Luminal patients have very low response rates to dual-anti-HER2 therapy but may respond better to combination therapy including an AKT-inhibitor. HR-positivity, though very important in general for determining who should receive adjuvant endocrine therapy, is not used in this response-predictive schema, as further subdivisions based on HR-status would not impact agent prioritization. In our in silico experiment, treatment assignment based on matching HR/HER2 subsets to the most effective therapy improves trial level pCR from 19% to 51%; and assignment based on RPS-5 added a further 7% improvement to 58% pCR.

More generally, we showed that molecular subtyping categories incorporating biology outside HR/HER2 could be created and that these new categories can better inform treatment assignment to new emerging therapies for breast cancer for individual patients and increase efficacy (i.e. pCR rate) over the entire treatment population. However, when comparing the relative contributions of improved biomarkers vs improved agents to response rate over the entire trial population, we observe that most of the pCR benefit appears to derive from the ‘right’ treatments (+30%) and an additional sizable pCR benefit comes from improved biomarker schemas (<=10-15%). With current agents, the highest pCR rate over the I-SPY2 population appears capped at ˜65% in the best performing schemas incorporating Immune, Luminal and HER2-3state biomarkers. This limitation likely derives from a sizeable patient population with luminal biology who are Immune-negative and DRD-negative who did not respond to any of the treatments under study. Many of these patients are predicted endocrine responsive and may benefit from neoadjuvant endocrine therapy, an approach we are considering testing in the future.

We observe that different schemas have different sets of ‘best’ treatments, with some treatments (e.g., Pembro) chosen by all schemas, and others by a subset of schemas or not at all, although that is partially a consequence of the biological phenotypes included. As new agent classes that may help further improve response rate over the population become available, we will need to incorporate new biological phenotypes into existing subtyping schemas that only classify cancers optimally for existing agents. Using HER2low-targeted agents as an example (an agent in this class is currently in I-SPY2), we developed a new schema incorporating HER2 status as a 3-state variable (HER2−0, HER2-low, HER2+), and the resulting treatment Response Predictive Subtype 7 (RPS-7) classification further improved pCR rates in the overall population in our in silico experiments. This example also illustrates that the minimum efficacy required to demonstrate benefit (over best available agent) differs by biomarker subsets.

It is important to note that we make a distinction between predictive biological phenotypes like ‘Immune+’ and their implementation. For instance, in our study Immune+ is, based on a variety of different subtype-specific signatures (e.g. B cell signature in HR+, STAT1/chemokine signature in TN). The implementation we selected in this study will be translated to a single-sample predictor for implementation in a clinical setting. CLIA compliant, clinically actionable versions of some of our selected biomarkers have been developed and an IDE submission is underway to enable prospective testing in the next-generation ‘I-SPY2.2’ trial. However, the idea is that as new, improved biomarkers are developed, the best available can be ‘swapped in’ to implement the phenotype in the clinic.

The ISPY2-990 Data Resource, and our analyses, have limitations. Each arm is relatively small (44-120 patients); further dividing these groups by receptor subtype or by one of the new response-predictive subtyping schemas, the numbers become even smaller, and the cohort sizes are unequal. This limits the power of analysis. In addition, I-SPY2 uses adaptive randomization within HR/HER2/MP defined subtypes to enable efficient matching of treatment regimens with their most responsive traditional clinical subtypes. This may result in the unbalanced prevalence of biomarker-positive subsets in experimental and control arms if a biomarker subset is correlated with a HR/HER2/MP subset that is preferentially enriched or depleted in an experimental arm by the randomization engine. For combination therapies (e.g. VC and TDM1/P) it is impossible to tease out relative contributions of each agent to response or to assess whether a biomarker is predictive of response to the individual agents within the combination. Thus, the statistics described in these examples are descriptive.

Another limitation to our underlying biomarker data is that potential platform “batch” effects may not be possible to entirely eliminate or correct for algorithmically. Also, RPPA data is not available for all patients. The tissue assayed for RPPA analysis in this study is derived from LCM-enriched tumor epithelium, and therefore does not fully capture elements of the tumor microenvironment such as stromal immune infiltration. Moreover, while we utilized a multi-omic biomarker approach to generate multiplexed RNA-protein-phosphoprotein data as well as CLIA-based platforms, the study is limited to having only two biomarker platforms, and by the selection of the short list of continuous qualifying biomarkers as the focus. For instance, we cannot include some well-studied biomarkers, such as HRD and other DNA ‘scar’ assays for DNA repair deficiency, which requires DNA sequencing data, and we do not include exploratory whole-transcriptome or whole-RPPA array analyses.

In conclusion, we found biomarkers predictive of response to a variety of agents with different mechanisms of action and proposed a framework for identifying a response-predictive subtyping schema for prioritizing therapies. Within this framework, we provide a clinically relevant breast cancer classification schema incorporating immune, DRD, and luminal-like biological phenotypes and new approaches to defining HER2 status to improve agent prioritization for individual patients and increase pCR rates over the population.

Immune Biomarkers as Defined for Immune Therapy Response in Four Additional Arms.

We showed above that in the pembrolizumab (Pembro) arm of I-SPY2, pCR associates with high STAT1/chemokine/dendritic signatures in TN and with high B-cell/low mast cell in HR+. From these results, we defined a research-grade Immune classifier incorporated into the RPS (PMID: 35623341), a schema designed to increase pCR if used to prioritize treatment. A clinical-grade version of the Immune (ImPrint) and other RPS biomarkers are now used in I-SPY2. Here we evaluate immune markers in 5 IO arms (Pembro, Durvalumab/Olaparib (Durva), Pembro/SD101, Cemiplimab (Cemi), and Cemi/fianlimab(LAG3)).

Methods: 343 patients with HER2-negative BC with information on pCR and mRNA in 5 IO arms (Pembro: 69, Durva: 71, Pembro/SD101:72, Cemi: 60, Cemi/LAG3: 71) plus controls (Ctr: 343) were considered. 32 continuous markers including 30 immune (7 checkpoint genes, 14 immune cell, 3 T/B-cell prognostic, 1 TGFB and 5 tumor-immune) and ESR1/PGR and proliferation signatures, were assessed for association with pCR using logistic regression. p-values were adjusted using the Benjamini-Hochberg method (BH p<0.05). Correlations to multiplex immunofluorescence (mIF) data from Pembro (immune cell and spatial proximity markers) were calculated. Performance of ImPrint, developed with Agendia Inc, was characterized overall and within HR subsets. Describes different treatments controls figure with little red circles something with Denis now include figures with red and blue cirecles

Results: A larger number of the research-grade immune markers predict response to IO in HR+ than in TN, with the most for HR+ in combination-IO arms (27/32 Pembro/SD101 and 17/32 Cemi/LAG3).

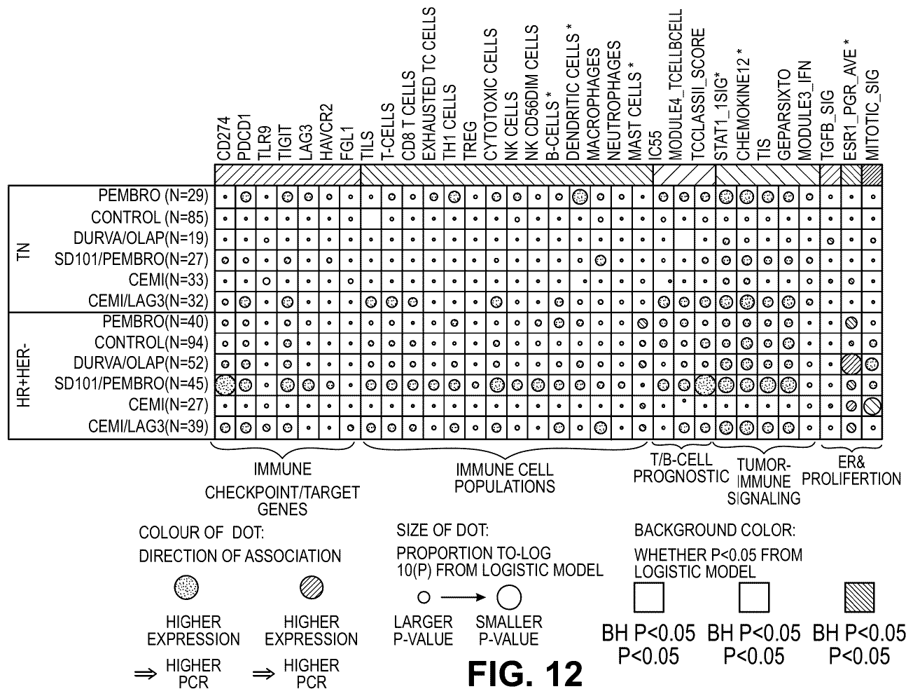

Tumor-immune signatures dominated by chemokines/cytokines were most consistently associated with pCR across IO arms and across receptor status (FIG. 12). Moreover, we found that these markers correlate to mIF spatial proximity measures reflecting high spatial co-localization of PD1+ immune and PDL1+ tumor cells, in TN especially (r=0.59; p=0.003).

The ImPrint classifier was evaluated in the IO arms. In HR+, 28% were ImPrint+; and pCR rates were 76% in ImPrint+vs. 16% in ImPrint-. In TN, 46% were ImPrint+; and pCR rates were 75% in ImPrint+ and 37% in ImPrint-.

Overall (HR+ and TN, in all IO arms), pCR rates were 75% in ImPrint+ and 23% in ImPrint-. Performance varied by arm, with the highest pCR rates for HR+/ImPrint+ in Durva and Cemi/LAG3 (>90%); and for TN/ImPrint+ in Cemi and Cemi/LAG3 (>81%). In contrast, pCR rates in the control arm were 34% for ImPrint+(HR+:33%; TN: 34%) and 13% for ImPrint-(HR+: 21%; TN:8%).

The analyses provided above demonstrate that tumor-immune signaling signatures predict IO response in both TN and HR+HER2−. The ImPrint single-sample classifier predicts response to a variety of IO regimens in both subsets and may inform prioritization of IO vs other treatments and best balance likely benefit vs risk of serious immune-related adverse events.

Experimental Model and Subject Details Defining RPS

I-SPY2 TRIAL Overview

Transcriptomic, protein/phospho-protein and clinical data used in this study will be available in NCBI's Gene Expression Omnibus (GEO) ([GEO IDs—record in progress]) and through the I-SPY2 Google Cloud repository for ispytrials.org/results/data).

I-SPY2 is an ongoing, open-label, adaptive, randomized phase II, multicenter trial of neoadjuvant therapy for early-stage breast cancer (NCT01042379; IND 105139). It is a platform trial evaluating multiple investigational arms in parallel against a common standard of care control arm. The primary endpoint is pCR (ypT0/is, ypN0), defined as the absence of invasive cancer in the breast and regional nodes at the time of surgery. As I-SPY2 is modified intent-to-treat, patients receiving any dose of study therapy are considered evaluable; those who switch to non-protocol therapy, progress, forgo surgery, or withdraw are deemed ‘non-pCR’. Secondary endpoints include residual cancer burden (RCB) and event-free and distant relapse-free survival (EFS and DRFS) (Symmans et al., 2007)

Trial Design

Assessments at screening establish eligibility and classify participants into subtypes defined by hormone receptor (HR) status, HER2, and 70-gene signature (MammaPrint®) status (Cardoso et al., 2016; Piccart et al., 2021). Adaptive randomization in I-SPY2 preferentially assigns patients to trial arms according to continuously updated Bayesian probabilities of pCR rates within each biomarker signature; 20% of patients are randomly assigned to the control arm (Berry, 2011). While accrual is ongoing, a statistical engine assesses the accumulating pathologic and MRI responses at weeks 3 and 12 and continuously re-estimates the probabilities of an experimental arm being superior to the control in each defined biomarker signature. An arm can be dropped for futility if the predicted probability of success in a future 300-patient, 1:1 randomized, phase 3 trial drops below 10%, or graduate for efficacy if the probability of success reaches 85% or greater in any biomarker signature. The clinical control arm for the efficacy analysis uses patients randomized throughout the entire trial. Experimental arms have variable sample sizes: highly effective therapies graduate with fewer patients in the experimental arm; arms that are equal to, or marginally better than, the control arm accrue slower and are stopped if they have not graduated, or terminated for lack of efficacy, before reaching a sample size of 75. During the design of each new experimental arm the investigators together with the pharmaceutical sponsor decide in which of the 10 a priori defined biomarker signatures the drug will be tested. Upon entry to the trial, participants are dichotomized into hormone receptor (HR) negative versus positive, HER2 positive versus negative, and MammaPrint High1 [MP1] versus High2 [MP2] status. From these 8 biomarker combinations (2×2×2) I-SPY has created 10 biomarker signatures that represent the disease subsets of interest (e.g. all patients, all HR+, all HER2+, HR+/HER2, etc., for complete list see reference Berry 2011) in which a drug can be tested for efficacy.

Efficacy is monitored in each of these 10 biomarker signatures separately and an arm could graduate in any or all biomarker signature of interest. When graduation occurs, accrual to the arm stops, final efficacy results are updated when all pathology results are complete. The final estimated pCR results therefore may differ from the predicted pCR rate at the time of graduation. Additional details on the study design have been published elsewhere. (Park et al., 2016; Rugo et al., 2016)

Eligibility

Participants eligible for I-SPY2 are women>18 years of age with stage II or III breast cancer with a minimum tumor size of >2·5 cm by clinical exam, or >2·0 cm by imaging, and Eastern Cooperative Oncology Group performance status of 0 or 1 (Oken et al., 1982). HR-positive/HER2-negative cancers assessed as low risk by the 70-gene MammaPrint test are ineligible as they receive little benefit from systemic chemotherapy.

Treatment

This correlative study involved 987 women with high-risk stage II and III early breast cancer who were enrolled in 10 arms of I-SPY2: the first 9 experimental arms that completed evaluation and the control arm as shown in the schema of FIG. 1A. During this same period (2010-2017), one arm was stopped due to toxicity with few patients enrolled and is not included in this evaluation. All patients received at least standard chemotherapy (paclitaxel alone followed by doxorubicin/cyclophosphamide (T→AC; or with trastuzumab (H) in HER2+, T+H→AC)) or in combination (taxane phase) with investigational agents: veliparib/carboplatin (VC; HER2− only: VC→AC); neratinib (N; All patients: T+N→AC); MK2206 (M; HER2−: T+M→AC; HER2+: T+H+M→AC); ganitumab (HER2− only: T+GM→AC); ganetespib (HER2− only: T+GS→AC); trebananib (HER2−: T+trebananib→AC; HER2+: T+H+AMG386→AC); TDM1/pertuzumab (P) (HER2+: TDM1/P→AC); pertuzumab (HER2+: T+pertuzumab→AC); and pembrolizumab (Pembro; HER2−: T+Pembro→AC). For HER2+ patients, N was administered instead of H, whereas M and trebananib were administered in addition to H. Dose reductions and toxicity management were specified in the protocol. Adverse events were collected according to the NCI Common Terminology Criteria for Adverse Events (CTCAE) version 4.0. After completion of AC, patients underwent lumpectomy or mastectomy and nodal sampling, with choice of surgery at the discretion of the treating surgeon. Detailed descriptions of the design, eligibility, and efficacy of these 9 experimental arms of the I-SPY2 trial have been reported previously (Chien et al., 2019; Clark et al., 2021; Nanda et al., 2020; Park et al., 2016; Pusztai et al., 2021; Rugo et al., 2016).

Trial Oversight

I-SPY2 is conducted in accordance with the guidelines for Good Clinical Practice and the Declaration of Helsinki, with approval for the study protocol and associated amendments obtained from independent ethics committees at each site. Written, informed consent was obtained from each participant prior to screening and again prior to treatment. The I-SPY2 Data Safety Monitoring Board meets monthly to review patient safety.

Method Details

Pretreatment Biopsy Processing and Molecular Profiling

Core needle biopsies of 16-gauge were taken from the primary breast tumor before treatment. Collected tissue samples are immediately frozen in Tissue-Tek® O.C.T.™ embedding media and then stored in −80° C. until further processing. An 8 μM section is stained with hematoxylin and eosin (H&E) and pathologic evaluation performed to confirm the tissue contains at least 30% tumor. A tissue sample meeting the 30% tumor requirement is further cryosectioned at 30 μM. Twenty to thirty sections are collected and emulsified in 0.5 ml Qiazol solution and the tubes are sent on dry ice to Agendia, Inc., for RNA extraction and gene expression profiling on Agilent 44K (GPL16233; n=333) or 32K (GPL20078; n=654) expression arrays. For each array, the green channel mean signal was log 2—tranformed and centered within array to its 75^{th} quantile as per the manufacturer's data processing recommendations. All values indicated for non-conformity are NA'd out; and a fixed value of 9.5 was added to avoid negative values. Probeset level data per array were mean-collapsed to the gene level, and genes common to the two platforms identified. Expression data from the first ˜900 I-SPY2 patients distributed over the two platforms GPL16233 (n=333) and GPL20078 (n=545) were combined into a single gene-level dataset after batch-adjusting using ComBat (Johnson et al., 2007). Linear adjustment factors were derived from the larger ComBat operation, per platform, which can be used to batch correct raw files. The subsequent ˜90 samples, assayed on GPL20078, were batch corrected using these factors and added to the original set, yielding a normalized expression dataset comprising 987 patients x 19,134 (common) genes. These transcriptomic data and the associated batch correction model coefficients are available in NCBI's Gene Expression Omnibus (GEO) [GEOID pending] and through the I-SPY2 Google Cloud repository (see, www site ispytrials.org/results/data).

In addition, laser capture microdissection (LCM) was performed on pre-treatment biopsy specimens to isolate tumor epithelium for signaling protein and phospho-protein profiling by reverse phase protein arrays (RPPA) in the Petricoin Lab at George Mason University, as previously published [ref]. Approximately 10,000 cells are captured per sample. RPPA samples were assayed on three arrays, each containing hundreds of samples from different arms of the trial quantifying up to 140 protein/phospho-protein endpoints (GPL28470). To remove batch effects we standardized each array prior to combining, by (1) sampling 5000 times, maintaining a receptor subtype balance equal to that of the first ˜1000 patients (HR+HER2−: 0.384, TN:0.368, HR+HER2+:0.158, HR-HER2+:0.09); (2) calculating the mean(mean) and mean(sd) for each RPPA endpoint; (3) z-scoring each endpoint using the calculated mean/sd from (2). The consort diagram with the number of evaluable patients for each molecular profiling analysis is shown in FIG. 1B. Details of the RPPA sample preparation and data processing are as previously described (Wulfkuhle et al., 2018). These RPPA data for 736 patients (all arms except ganitumab and ganetespib) are available in NCBI's Gene Expression Omnibus (GEO) [GEOID pending] and through the I-SPY2 Google Cloud repository (available at website ispytrials.org/results/data).

Continuous Gene Expression Biomarkers Assessed

Twenty-six prospectively defined, mechanism-of-action and pathway-based expression and protein/phospho-protein continuous signatures assayed from pre-treatment biopsies were previously found to be predictive in a particular agent/arm in pre-specified QBE analysis. We also include an exploratory VC-response signature for the TN subset reflecting both DNA repair deficiency and Immune expression that validated in BrighTNess and therefore achieved qualifying status, for a total of 27 continuous biomarkers considered in our analysis (see Table 1 for genes/proteins included per signature and scoring method).

VCpred_TN derivation: VCpred_TN is a continuous gene expression signature that associates with response to VC in the TN subset. It differs from the other biomarkers in this study in that it was originally developed on I-SPY2 data, rather than previously published and in pre-specified analysis validated (qualified) in I-SPY2. We developed this signature in 2018, when the decision was made to switch I-SPY2 tumor biopsy tissue collection from fresh frozen (FF) as assayed for the I-SPY2-990 data compendium, to FFPE, and after performing expression studies of 72 matched FF:FFPE pairs from I-SPY2 that suggested that the previous DRD biomarker implementation frontrunner, PARPi7, may not translate well. In a quest to develop a more robust DRD biomarker that might better translate from FF to FFPE and between Agilent 44K platforms (GPL16233 and GPL20078) we developed VCpred_TN by: 1) collecting a large set of DNA repair related genes (Knijnenburg et al., 2018) including those in the PARPi7, and adding to them a subset of immune genes from module4 (Wolf et al., 2014) and IR7 (Teschendorff and Caldas, 2008), ESR1, and PGR, for a total of 162 genes; 2) filtering those 162 genes for presence on both Agilent 44K array types used in this study and for correlation between FF and FFPE samples using our 72-paired sample set (pearson correlation>0.4), which yielded an 84 gene starting set for signature development; and 3) assessing association between expression levels of each of the 84 genes and pCR in the VC arm, in the TN subset using logistic modeling, after mean-centering the expression data. The resulting signature is the sum of −sign(coeff)*log(p) for the top 25 most correlated genes in the starting set, where sign(coeff) the sign of association between a gene and pCR (positive if higher levels associate with pCR, negative if higher levels associate with non-pCR), and p=the likelihood ratio p-value. As also appears in the above Table 1, VCpred_TN=13.60*CXCL13−6.48*BRCA1+6.41*APEX1+5.32*FEN1+4.85*CD8A−4.84*SEM1+4.78*APEX2−4.60*RNMT+4.51*CCR7+3.99*H2AFX+3.88*POLD3−3.49*PRKDC+3.48*C1QA+3.33*CLIC5−3.24*RAD51+3.10*DDB2−2.83*SPP1−2.80*POLD2−2.80*POLB+2.72*LIGT−2.67*GTF2H5−2.63*PMS2+2.60*LY9−2.34*SHPRH+6.27*ARAF; where the expression data is mean-centered by gene over all samples prior to evaluating this weighted sum, and the final signature is z-scored to have mean=0 and sd=1.

Biological Response-Predictive Phenotypes: Overview and Implementation

Here we introduce the concept of and response-predictive biological phenotype, defined by considering promising treatments (e.g. Immunotherapy, dual-HER2, and platinum-based) and basic cancer biology (e.g. proliferation). Patients are considered Immune-positive (Immune+) if their immune-tumor state is such that they are likely to respond to immunotherapy, and DNA repair deficient/platinum-responsive (DRD+) if response to a platinum agent with or without PARP-inhibition is likely. As biomarkers representing the same biology are correlated and can be subtype-specific (FIG. 2), multiple immune and DRD markers can be used to implement these biological phenotypes and perform similarly. Moreover, though we need to select example implementations for response predictive phenotypes like Immune, HER2ness, Luminal, DRD, and proliferation, we do so with the expectation that as alternative biomarkers come available, they can be ‘swapped in’.

In general, we prefer to use categorical biomarkers, so as to not have to select thresholds using I-SPY2 trial data. Here we use BluePrint subtype (Agendia; BP-Luminal, BP-Her2, BP-Basal) to implement Her2ness, Luminal and Basal biological phenotypes, and MP2 class as a proliferation biomarker based on high levels of correlation to cell cycle/proliferation signatures.

Where necessary, we also dichotomize continuous biomarkers using a subtype-specific cross-validation procedure to optimize performance as follows:
Biomarker dichotomization: To identify optimal (exploratory) dichotomizing thresholds for select biomarkers in a particular patient subset, a cross-validation procedure was applied to selected endpoints associated with pCR in a selected treatment arm of the trial to identify potential cut points for biomarker positivity. Two-fold cross-validation was repeated 1000 times, with test and training sets balanced over pCR, using logistic models to assess association with response. A cutpoint was selected as ‘optimal’ if: (1) it was selected as optimal>100 times in the training set; (2) p<E-15 in the test sets (combined using the logit method (Dewey, 2018)); and (3) the prevalence is reasonably balanced.

Immune phenotype: example implementation: Patients are considered Immune− positive (Immune+) if their immune-tumor state is such that they are likely to respond to immunotherapy. In general, immune signatures are correlated, therefore there are many possible implementations that may perform similarly. In this study we use a subtype-specific implementation. Based on our qualifying biomarker analysis, for TN patients we used the average of the dendritic cell and STAT1 signatures (Danaher et al., 2017; Rody et al., 2009; Yau et al., 2019). These biomarkers were the top two most predictive of TN response to pembrolizumab in this study (FIG. 3) and the STAT1 signature has been further validated in the previously published durvalumab/olaparib arm of I-SPY2 (Pusztai et al., 2021) and in an independent Phase II trial (NCT02489448) (Blenman et al., 2020; Foldi et al., 2021; Pusztai et al., 2021). Specifically, we (1) z-scored their average ((STAT1_sig+Dendritic_sig)/2, denoted STAT1_Dendritic_ave), and (2) optimally dichotomized the averaged signatures per above using pCR data from the Pembro arm, yielding a cutpoint of 0 (TN/Immune-high: STAT1_Dendritic_ave>=0; and TN/Immune-low: STAT1_Dendritic_ave<0).

In the HR+HER2-subset, high B-cell and low mast-cell immune gene signatures were strong predictors of pCR to immunotherapy (FIG. 3) and we use them in dichotomized form as an example implementation for our Immune+ phenotype in this subset. This choice was based on the observation that to achieve high predictive accuracy in the HR+HER2− subset, it is necessary to combine a ‘sensitivity’ immune biomarker (e.g. Bcell) with a second ‘resistance’ biomarker where high levels predict non-pCR (either Mast-cell or ESR1/PGR averaged). Applying the above dichotomization procedure yielded cutpoints 0.1495 for Bcell_score and 1.17 for MastCell_score (HR+HER2−/Immune-high: (B_cells>=0.1495) AND (Mast_cells<1.17); HR+HER2−/Immune-low: (B_cells<0.1495) OR (Mast_cells>=1.17)).

For HER2+ patients, we optimally dichotomized the B_cells signature in the combined MK2206, control and neratinib arms where immune signals associate with response, yielding a cutpoint of 0.58 (HER2+/Immune-high: B_cells>=0.58; HER2+/Immune-low: B_cells<0.58).

DRD phenotype: example implementation: Our implementation of the DRD response-predictive phenotype is also subtype-specific. In the TN subset, we had intended to use the previously described PARPi7 gene signature (FIG. 3; (Daemen et al., 2012; Wolf et al., 2017)) as an example implementation, but it did not validate in the BrighTNess trial (Filho et al., 2021; Loibl et al., 2018) (p>0.05). Instead we used the VCpred_TN signature developed in I-SPY2 (see above and Table 1), which validated in BrighTNess (p=5.08E-06) (FIG. 9C). We dichotomized the VCpred_TN using pCR data from the VC arm, using the above-described cross-validation optimization procedure and also taking into account our intention of using this biomarker in a multi-agent context with immunotherapy and an immune biomarker. Though the optimal cutpoint if only considering performance in VC is 0.35, this threshold results in a clinically important subset defined by Immune−/DRD+ that is too small (4%) to be clinically reasonable. Therefore we chose a ‘next best’ cutpoint of −0.31 (TN/DRD+: VCpred_TN>(−0.31); TN/DRD−: VCpred_TN<(−0.31)). With this cutpoint, the Immune−/DRD+ subset is a more clinically actionable size at 110%.

We used BP-Basal classification as our measure to assess the DRD phenotype in HR+HER2− (HR+HER2−/DRD+: BP_Basal; HR+HER2−/DRD−: BP_Luminal) because the assay is performed in a CLIA setting and is ready for clinical implementation with a pending IDE application submission to the US FDA, even though the research assay based PARPi7-high/MP2 performed somewhat better in this dataset (Daemen et al., 2012; Wolf et al., 2017).

Three-state clinical HER2 status: When considering a new HER2low-targeted agent, we used HER2 IHC levels (3+, 2+, 1+, 0) and HER2 FISH to define a 3-class clinical HER2 biomarker HER2-3state (HER2=0: IHC 0 and FISH−; HER2low: IHC 2+/1+ and FISH−; and HER2+: IHC 3+ or FISH+ as currently defined in the trial).

Combining Response-Predictive Phenotypes and HR/HER2 Status into Response-Predictive Subtyping Schemas

Once multiple response-predictive phenotypes are added to HR and HER2 status, there is a combinatorial explosion in the number of possible states, and many ways to collapse them into a practical number of subtypes (<8 or 9). To sort through the options, we reasoned that an ideal response-predictive subtyping schema should: R1) differentiate between treatments, meaning that different classes should have different best treatments yielding the highest pCR probability; R2) result in a higher pCR rate in the population if used to optimally assign/prioritize treatments; R3) differentiate between responders and non-responders over a wide range of treatment classes; and R4) be robust to platform and within-class treatments, simple to implement, and FDA approved or performed in a CLIA environment. For (R1) we generalize the ‘Carnaugh Map’ method used in circuit design to simplify digital logic (Brown, 1990). For example, if HR+HER2−/Immune−/DRD+ and TN/Immune−/DRD+ classes both have VC as the treatment yielding the highest pCR rate, we collapse them into a single class HER2−/Immune−/DRD+ as seen in FIGS. 5A-5C.

Implementation of Previously Published PAM50 and TNBC-4Class and -7Class Subtyping Schemas

In addition to standard clinical variables like HR, HER2, MP, pCR and Arm, several biomarker heatmaps (e.g., FIG. 2) are annotated for PAM50 and two TNBC classification schemas as well, evaluated as previously described. PAM50 intrinsic subtyping was performed using Joel Parker's centroid-based 50-gene classifier program (Parker et al., 2009) on a total of 1151 samples including 165 in the I-SPY low-risk registry (open to those who screen out of I-SPY2 due to assessment of low molecular risk by the 70-gene MammaPrint test). We included the low-risk registry patients in the dataset (mostly HR+HER2− Luminal A) prior to subtyping because I-SPY2 HR+HER2− patients are all MP high risk (mostly Luminal B) and we wanted the population to be more representative of the general breast cancer patient population as is required for sensible results. We also centered the genes on the mean value of repeated subsampling (500 times) of 1:1 ER+:ER− prior to running the code, as previously advised by Katie Hoadley (private communication) to obtain classifications most consistent with their original paper. Finally, we set to NA any call with a confidence level<0.08, of which there were 14. TNBCtype classifications (7 classes: MSL, M, LAR, IM, BL2, BL1) were identified as published (Chen et al., 2012; Lehmann et al., 2011) by uploading (non-median centered) expression data from the TN subset (n=363) to the online calculator (https site cbc.app.vumc.org/tnbc/). The Burstein/Brown TN classifications (LAR, MES, BLIS, BLIA) were identified as published (Burstein et al., 2015), by: (1) quantile transforming over their predictor genes; (2) calculating Euclidean distance to the 4 published centroids; and (3) assigning class based on the closest (minimal distance) centroid.

Methodology—Quantification and Statistical Analysis

Statistical Analysis of Continuous Gene Expression Biomarkers

We assessed association between each continuous biomarker and response in the population as a whole and within each arm and HR/HER2 subtype using a logistic model. In whole-population analyses, models are adjusted for HR, HER2, and treatment arm (pCR-biomarker+HR+HER2+Tx). Within treatment arms, models are adjusted for HR and HER2 as appropriate. Markers are analyzed individually; likelihood ratio (LR) test p-values are descriptive.

We also performed exploratory whole transcriptome and whole RPPA dataset analysis, per above, employing Benjamini-Hochberg multiple testing correction (Huang et al., 2009), with a significance threshold of BH p<0.05. Analyses and visualizations were performed in the computing environment R (v.3.6.3) using R Packages ‘stats’ (v.3.6.3), ‘lmtest’ (v.0.9-37), ‘rjags’ (v.4-10) and ‘GoogleVis (v.0.6.4). Scripts are available upon request.

Response-Predictive Subtyping Schema Characterization

For each subtype/class in each schema, we calculated pCR rates in each arm with sufficient patients and displayed the results (100*(number of patients with pCR)/total) in bar plots. A major goal of a response-predictive schema is to increase the pCR rate in the population and to maximize the probability of pCR for an individual patient (R2). To characterize the potential impact of the new classification, we calculated the overall pCR rate in the I-SPY2 population had treatments been optimally assigned according to the new subtypes using the same 10 drugs. To this end, we: (1) calculated the prevalence of each subtype in the schema (prev_STi=(number of patients in STi)/(total number of patients), i=1:n, n=number of subtypes); (2) collected highest-pCR rates observed in an I-SPY2 arm for each subtype (pCR_max_STi); and (3) calculated a simple estimate of the pCR rate over the population as the weighted sum pCR_max total=prev_ST1*pCR_max_ST1+prev_ST2*pCR_max_ST2+ . . . prev_STn*pCR_max_STn. This calculation results in both an estimate of pCR over the population using the new schema, and identification of agents/combinations maximizing pCR for each subtype.

To characterize the pCR-predictive power of a subtyping schema within an arm (R3), we use bias corrected mutual information (BCMI; R package mpmi http://r-forge.r-project.org/projects/mpmi/), which quantifies the amount of uncertainty reduced about pCR by knowing subtype. These values are then visualized across arms in a scatter plot with BCMI and pCR-association p-values (LR p) on the axis, for both receptor subtype and a response-predictive subtyping schema to visualize differences. In addition, we used Fisher's exact test for associations with response, and Cox proportional hazards modeling to estimate hazard ratios for pCR within each RPS-5 subtype using the coxph and Surv functions within the R package survival.

RESOURCES TABLE

| REAGENT or | SOURCE | IDENTIFIER |
|---|---|---|
| Biological samples | | |
| Tumor biopsy before | I-SPY2 TRIAL | website |
| treatment | | clinicaltrials.gov/ct2/show/NCT01042379 |

Critical commercial assays

| | | |
|---|---|---|
| Custom Agilent 44K | Agendia, Inc | Website |
| expression arrays | | ncbi.nlm.nih.gov/geo/query/acc.cgi?acc=GP |
| | | L20078; |
| | | Website |
| | | ncbi.nlm.nih.gov/geo/query/acc.cgi?acc=GP |
| | | L16233 |
| MammaPrint | Agendia, Inc | agendia.com mammaprint |
| BluePrint | Agendia, Inc | Agendia.com blueprint |
| Reverse phase protein | Petricoin Lab, George | website |
| array (RPPA) | Mason University | ncbi.nlm.nih.gov/geo/query/acc.cgi?acc=GP |
| | | L28470 |

Deposited data

| | | |
|---|---|---|
| Raw and processed | This study | website/console.cloud.google.com/storage/ |
| transcriptomic data | | browser/wolfet_al_2021_ispy2_subtypes_990 |
| | | GEO ID Pending |
| Raw and processed | This study | Website |
| RPPA data | | console.cloud.google.com/storage/ |
| | | browser/wolf_et_al_2021_ispy2_subtypes_990 |
| | | GEO ID Pending |
| Patient-level | This study | Website |
| expression signature | | console.cloud.google.com/storage/ |
| and clinical data | | browser/wolfet_al_2021_ispy2_subtypes_990 |
| | | GEO ID pending |

Software and algorithms

| | | |
|---|---|---|
| stats R package | R Core Team (2020) | Website stat.ethz.ch/R-manual/R-devel/ |
| (v.3.6.3) | | library/stats/html/stats-package.html |
| lmtest R package | Zeileis A, Hothorn T | Website CRAN.R-project.org/package=lmtest |
| (v.0.9-37) | (2002). “Diagnostic | |
| | Checking in Regression | |
| | Relationships.” R News, | |
| | 2(3), 7-10. | |
| rjags R package | Martyn Plummer (2019). | Website CRAN.R-project.org/package=rjags |
| (v.4-10) | rjags: Bayesian | |
| | Graphical Models using | |
| | MCMC. R package v4-10. | |
| googleVis R package | Gesmann M, de Castillo | Website CRAN.R-project.org/package=googleVis |
| (v.0.6.4) | D (2011). “googleVis: | |
| | Interface between R and | |
| | the Google Visualisation | |
| | API.” The R Journal, | |
| | 3(2), 40-44 | |
| survival R package | Terry M. Therneau, Patricia | Website CRAN.R-project.org/package=survival |
| (v.3.1-12) | M. Grambsch (2000). | |
| | Modeling Survival Data: | |
| | Extending the Cox Model. | |
| | Springer, New York. ISBN | |
| | 0-387-98784-3. | |

It is understood that the examples and embodiments described in the present application are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference for the subject matter for which they are cited.

### References Cited in Application by Author and Year

Bergin, A. R. T., and Loi, S. (2019). Triple-negative breast cancer: recent treatment advances. F1000research 8, F1000 Faculty Rev-1342.
Berry, D. A. (2011). Adaptive clinical trials in oncology. Nature Reviews Clinical Oncology 9, 199-207.
Blenman, K. R. M., Li, X., Marczyk, M., O'Meara, T., Yaghoobi, V., Gunasekharan, V., Park, T., Rimm, D., and Pusztai, L. (2020). Abstract P3-09-05: Predictive markers of response to durvalumab concurrent with nab-paclitaxel and dose dense doxorubicin cyclophosphamide (ddAC) neoadjuvant therapy for triple negative breast cancer (TNBC). P3-09-05-P3-09-05.
Brown, F. M. (1990). Boolean reasoning: the logic of Boolean equations.
Burstein, M. D., Tsimelzon, A., Poage, G. M., Covington, K. R., Contreras, A., Fuqua, S. A., Savage, M. I., Osborne, C. K., Hilsenbeck, S. G., Chang, J. C., et al. (2015). Comprehensive genomic analysis identifies novel subtypes and targets of triple-negative breast cancer. Clin Cancer Res 21, 1688-1698.
Cardoso, F., Veer, L. J. van′t, Bogaerts, J., Slaets, L., Viale, G., Delaloge, S., Pierga, J. Y., Brain, E., Causeret, S., DeLorenzi, M., et al. (2016). 70-Gene Signature as an Aid to Treatment Decisions in Early-Stage Breast Cancer. New England Journal of Medicine 375, 717-729.
Chen, X., Li, J., Gray, W. H., Lehmann, B. D., Bauer, J. A., Shyr, Y., and Pietenpol, J. A. (2012). TNBCtype: A Subtyping Tool for Triple-Negative Breast Cancer. Cancer Informatics 11, 147-156.
Chien, A. J., Tripathy, D., Albain, K. S., Symmans, W. F., Rugo, H. S., Melisko, M. E., Wallace, A. M., Schwab, R., Helsten, T., Forero-Torres, A., et al. (2019). MK-2206 and Standard Neoadjuvant Chemotherapy Improves Response in Patients With Human Epidermal Growth Factor Receptor 2-Positive and/or Hormone Receptor-Negative Breast Cancers in the I-SPY 2 Trial. J Clin Oncol 38, 1059-1069.
Clark, A. S., Yau, C., Wolf, D. M., Petricoin, E. F., Veer, L. J. van't, Yee, D., Moulder, S. L., Wallace, A. M., Chien, A. J., Isaacs, C., et al. (2021). Neoadjuvant T-DM1/pertuzumab and paclitaxel/trastuzumab/pertuzumab for HER2+ breast cancer in the adaptively randomized I-SPY2 trial. Nat Commun 12, 6428.
Daemen, A., Wolf, D. M., Korkola, J. E., Griffith, O. L., Frankum, J. R., Brough, R., Jakkula, L. R., Wang, N.J., Natrajan, R., Reis-Filho, J. S., et al. (2012). Cross-platform pathway-based analysis identifies markers of response to the PARP inhibitor olaparib. Breast Cancer Res Tr 135, 505-517.
Danaher, P., Warren, S., Dennis, L., D'Amico, L., White, A., Disis, M. L., Geller, M. A., Odunsi, K., Beechem, J., and Fling, S. P. (2017). Gene expression markers of Tumor Infiltrating Leukocytes. J Immunother Cancer 5, 18.
DeSantis, C. E., Bray, F., Ferlay, J., Lortet-Tieulent, J., Anderson, B. O., and Jemal, A. (2015). International Variation in Female Breast Cancer Incidence and Mortality Rates. Cancer Epidemiol Biomarkers Prev 24, 1495-1506.
Dewey, M. (2018). metap: meta-analysis of significance values. R Package Version 1.0.
Filho, O. M., Stover, D. G., Asad, S., Ansell, P. J., Watson, M., Loibl, S., E., Jr. G., C., Bae, J., Collier, K., Cherian, M., et al. (2021). Association of Immunophenotype With Pathologic Complete Response to Neoadjuvant Chemotherapy for Triple-Negative Breast Cancer: A Secondary Analysis of the BrighTNess Phase 3 Randomized Clinical Trial. JAMA Oncol 7, 603-608.
Foldi, J., Silber, A., Reisenbichler, E., Singh, K., Fischbach, N., Persico, J., Adelson, K., Katoch, A., Horowitz, N., Lannin, D., et al. (2021). Neoadjuvant durvalumab plus weekly nab-paclitaxel and dose-dense doxorubicin/cyclophosphamide in triple-negative breast cancer. Npj Breast Cancer 7, 9.
Gonzalez-Ericsson, P. I., Wulfkhule, J. D., Gallagher, R. I., Sun, X., Axelrod, M. L., Sheng, Q., Luo, N., Gomez, H., Sanchez, V., Sanders, M., et al. (2021). Tumor-Specific Major Histocompatibility-II Expression Predicts Benefit to Anti-PD-1/L1 Therapy in Patients With HER2-Negative Primary Breast Cancer. Clin Cancer Res 27, 5299-5306.
Huang, D. W., Sherman, B. T., and Lempicki, R. A. (2009). Systematic and integrative analysis of large gene lists using DAVID bioinformatics resources. Nat Protoc 4, 44-57.
Johnson, W. E., Li, C., and Rabinovic, A. (2007). Adjusting batch effects in microarray expression data using empirical Bayes methods. Biostatistics 8, 118-127.
Kim, M., Park, J., Bouhaddou, M., Kim, K., Rojc, A., Modak, M., Soucheray, M., McGregor, M. J., O'Leary, P., Wolf, D., et al. (2021). A protein interaction landscape of breast cancer. Science 374, eabf3066.
Knijnenburg, T. A., Wang, L., Zimmermann, M. T., Chambwe, N., Gao, G. F., Cherniack, A. D., Fan, H., Shen, H., Way, G. P., Greene, C. S., et al. (2018). Genomic and Molecular Landscape of DNA Damage Repair Deficiency across The Cancer Genome Atlas. Cell Rep 23, 239-254 e6.
Krijgsman, O., Roepman, P., Zwart, W., Carroll, J. S., Tian, S., Snoo, F. A. de, Bender, R. A., Bernards, R., and Glas, A. M. (2012). A diagnostic gene profile for molecular subtyping of breast cancer associated with treatment response. Breast Cancer Res Tr 133, 37-47.
Lee, P., Zhu, Z., Wolf, D., Yau, C., Audeh, W., Glas, A., Swigart, L., Hirst, G., DeMichele, A., Investigators, I. S., et al. (2018). Abstract 2612: BluePrint Luminal subtype predicts non-response to HER2-targeted therapies in HR+/HER2+I-SPY 2 breast cancer patients. Cancer Research 78, 2612-2612.
Lehmann, B. D., Bauer, J. A., Chen, X., Sanders, M. E., Chakravarthy, A. B., Shyr, Y., and Pietenpol, J. A. (2011). Identification of human triple-negative breast cancer subtypes and preclinical models for selection of targeted therapies. J Clin Invest 121, 2750-2767.
Loibl, S., O'Shaughnessy, J., Untch, M., Sikov, W. M., Rugo, H. S., McKee, M. D., Huober, J., Golshan, M., Minckwitz, G. von, Maag, D., et al. (2018). Addition of the PARP inhibitor veliparib plus carboplatin or carboplatin alone to standard neoadjuvant chemotherapy in triple-negative breast cancer (BrighTNess): a randomised, phase 3 trial. Lancet Oncol 19, 497-509.
McAndrew, N. P., and Finn, R. S. (2020). Management of ER positive metastatic breast cancer. Semin Oncol 47, 270-277.
Nanda, R., Liu, M. C., Yau, C., Shatsky, R., Pusztai, L., Wallace, A., Chien, A. J., Forero-Torres, A., Ellis, E., Han, H., et al. (2020). Effect of Pembrolizumab Plus Neoadjuvant Chemotherapy on Pathologic Complete Response in Women With Early-Stage Breast Cancer. Jama Oncol 6, 676-684.
Oken, M. M., Creech, R. H., Tormey, D. C., Horton, J., Davis, T. E., McFadden, E. T., and Carbone, P. P. (1982). Toxicity and Response Criteria of the Eastern-Cooperative-Oncology-Group. American Journal of Clinical Oncology-Cancer Clinical Trials 5, 649-655.
Park, J. W., Liu, M. C., Yee, D., Yau, C., Veer, L. J. van t, Symmans, W. F., Paoloni, M., Perlmutter, J., Hylton, N. M., Hogarth, M., et al. (2016). Adaptive Randomization of Neratinib in Early Breast Cancer. New England Journal of Medicine 375, 11-22.
Parker, J. S., Mullins, M., Cheang, M. C. U., Leung, S., Voduc, D., Vickery, T., Davies, S., Fauron, C., He, X., Hu, Z., et al. (2009). Supervised Risk Predictor of Breast Cancer Based on Intrinsic Subtypes. J Clin Oncol 27, 1160-1167.
Piccart, M., Veer, L. J. van't, Poncet, C., Cardozo, J. M. N. L., Delaloge, S., Pierga, J. Y., Vuylsteke, P., Brain, E., Vrijaldenhoven, S., Neijenhuis, P. A., et al. (2021). 70-gene signature as an aid for treatment decisions in early breast cancer: updated results of the phase 3 randomised MINDACT trial with an exploratory analysis by age. Lancet Oncol.
Pusztai, L., Yau, C., Wolf, D. M., Han, H. S., Du, L., Wallace, A. M., String-Reasor, E., Boughey, J. C., Chien, A. J., Elias, A. D., et al. (2021). Durvalumab with olaparib and paclitaxel for high-risk HER2− negative stage II/III breast cancer: Results from the adaptively randomized I-SPY2 trial. Cancer Cell 39, 989-998.e5.
Rody, A., Holtrich, U., Pusztai, L., Liedtke, C., Gaetje, R., Ruckhaeberle, E., Solbach, C., Hanker, L., Ahr, A., Metzler, D., et al. (2009). T-cell metagene predicts a favorable prognosis in estrogen receptor-negative and HER2-positive breast cancers. Breast Cancer Res Bcr 11, R15.
Rugo, H. S., Olopade, 0.1., DeMichele, A., Yau, C., Veer, L. J. van t, Buxton, M. B., Hogarth, M., Hylton, N. M., Paoloni, M., Perlmutter, J., et al. (2016). Adaptive Randomization of Veliparib-Carboplatin Treatment in Breast Cancer. New England Journal of Medicine 375, 23-34.
Sayaman, R. W., Wolf, D. M., Yau, C., Wulfkuhle, J., Petricoin, E., Brown-Swigart, L., Asare, S. M., Hirst, G. L., Sit, L., O'Grady, N., et al. (2020). Abstract P1-21-08: Application of machine learning to elucidate the biology predicting response in the I-SPY 2 neoadjuvant breast cancer trial. Cancer Res 80, P1-21-08-P1-21-08.
Schmid, P., Cortes, J., Pusztai, L., McArthur, H., Kummel, S., Bergh, J., Denkert, C., Park, Y. H., Hui, R., Harbeck, N., et al. (2020). Pembrolizumab for Early Triple-Negative Breast Cancer. N Engl J Med 382, 810-821.
Spring, L. M., Fell, G., Arfe, A., Sharma, C., Greenup, R., Reynolds, K. L., Smith, B. L., Alexander, B., Moy, B., Isakoff, S. J., et al. (2020). Pathologic Complete Response after Neoadjuvant Chemotherapy and Impact on Breast Cancer Recurrence and Survival: A Comprehensive Meta-analysis. Clin Cancer Res 26, 2838-2848.
Symmans, W. F., Peintinger, F., Hatzis, C., Rajan, R., Kuerer, H., Valero, V., Assad, L., Poniecka, A., Hennessy, B., Green, M., et al. (2007). Measurement of Residual Breast Cancer Burden to Predict Survival After Neoadjuvant Chemotherapy. J Clin Oncol 25, 4414-4422.
Teschendorff, A. E., and Caldas, C. (2008). A robust classifier of high predictive value to identify good prognosis patients in ER-negative breast cancer. Breast Cancer Research 10, R73.
Waks, A. G., and Winer, E. P. (2019). Breast Cancer Treatment: A Review. JAMA 321, 288-300.
Wolf, D., Yau, C., Swigart, L., Hirst, G., Investigators, I. S., Asare, S., Schwab, R., Berry, D., Esserman, L., Albain, K., et al. (2018). Abstract 2611: Evaluation of ANG/TIE/hypoxia pathway genes and signatures as predictors of response to trebananib (AMG 386) in the neoadjuvant I-SPY 2 TRIAL for Stage II-III high-risk breast cancer. Cancer Research 78, 2611-2611.
Wolf, D. M., Lenburg, M. E., Yau, C., Boudreau, A., and Veer, L. J. van't (2014). Gene co-expression modules as clinically relevant hallmarks of breast cancer diversity. PLoS One 9, e88309.
Wolf, D. M., Yau, C., Sanil, A., Glas, A., Petricoin, E., Wulfkuhle, J., Severson, T. M., Linn, S., Brown-Swigart, L., Hirst, G., et al. (2017). DNA repair deficiency biomarkers and the 70-gene ultra-high risk signature as predictors of veliparib/carboplatin response in the I-SPY 2 breast cancer trial. Npj Breast Cancer 3, 31.
Wolf, D. M., Yau, C., Wulfkuhle, J., Brown-Swigart, L., Gallagher, R. I., Magbanua, M. J. M., O'Grady, N., Hirst, G., I-SPY2 Trial Investigators, Asare, S., et al. (2020a). Mechanism of action biomarkers predicting response to AKT inhibition in the I-SPY 2 breast cancer trial. Npj Breast Cancer 6, 48.
Wolf, D. M., Yau, C., Wulfkuhle, J., Brown-Swigart, L., Asare, S. M., Hirst, G. L., Sit, L., Perlmutter, J., Consortium, I. S. 2 T., Liu, M., et al. (2020b). Abstract P4-10-02: HER2 signaling, ER, and proliferation biomarkers predict response to multiple HER2-targeted agents/combinations plus standard neoadjuvant therapy in the I-SPY 2 trial. Cancer Res 80, P4-10-02-P4-10-02.
Wuerstlein, R., and Harbeck, N. (2017). Neoadjuvant Therapy for HER2-positive Breast Cancer. Rev Recent Clin Trials 12, 81-92.
Wulfkuhle, J. D., Yau, C., Wolf, D. M., Vis, D. J., Gallagher, R. I., Brown-Swigart, L., Hirst, G., Voest, E. E., DeMichele, A., Hylton, N., et al. (2018). Evaluation of the HER/PI3K/AKT Family Signaling Network as a Predictive Biomarker of Pathologic Complete Response for Patients With Breast Cancer Treated With Neratinib in the I-SPY 2 TRIAL. Jco Precis Oncol 2, 1-20.
Yau, C., Wolf, D., Campbell, M., Savas, P., Lin, S., Swigart, L., Hirst, G., Asare, S., Zhu, Z., Loi, S., et al. (2019). Abstract P3-10-06: Expression-based immune signatures as predictors of neoadjuvant targeted-/chemo-therapy response: Experience from the I-SPY 2 TRIAL of ^{˜}1000 patients across 10 therapies. Cancer Research 79, P3-10.
Yee, D., DeMichele, A. M., Yau, C., Isaacs, C., Symmans, W. F., Albain, K. S., Chen, Y. Y., Krings, G., Wei, S., Harada, S., et al. (2020). Association of Event-Free and Distant Recurrence-Free Survival With Individual-Level Pathologic Complete Response in Neoadjuvant Treatment of Stages 2 and 3 Breast Cancer: Three-Year Follow-up Analysis for the I-SPY2 Adaptively Randomized Clinical Trial. JAMA Oncol 6, 1355-1362.

TABLE 1

| | | | | Scoring | |
|---|---|---|---|---|---|
| | | | | method* | |
| | | | | *starting with | |
| | | | | normalized and | |
| | | | | combined | |
| Continuous | | | | transcriptome and | |
| biomarker | Pathway | Type | Genes/proteins | RPPA data | Publication |
| Module5_TcellBcell | Immune | mRNA | IGSF6, LILRB2, BTN3A3, UBD, | 1) Mean center, | PMID: 24516633 |
| | | | CXCL13, GNLY, CXCR6, CTSC, | 2) take modified | |
| | | | HCP5, PIM2, SP140, CCR7, | inner product | |
| | | | CTSS, CYBB, FCN1, TFEC, | with centroid as | |
| | | | SEL1L3, FYB, GBP1, LAMP3, | published and | |
| | | | ADAMDEC1, GPR18, ICOS, | described below | |
| | | | GPR171, GZMH, GZMB, GZMK, | (though | |
| | | | BIRC3, IFNG, IL2RG, IL15, | averaging would | |
| | | | IDO1, CXCL10, IRF1, ISG20, | yield similar | |
| | | | ITK, LAG3, LCK, LYN, CXCL9, | results), | |
| | | | NKG7, TRAT1, MGC29506, | 3) Z-score | |
| | | | PLAC8, POU2AF1, CRTAM, | | |
| | | | SLAMF8, PSMB9, PTPN7, | | |
| | | | SLAMF7, BCL2A1, TNFRSF17, | | |
| | | | CCL5, CCL8, CCL13, CCL18, | | |
| | | | CCL19, CXCL11, SELL, SAMSN1, | | |
| | | | RTP4, CLEC7A, TAP1, WARS, | | |
| | | | PLA2G7, ZBED2, NPL, RUNX3, | | |
| | | | VNN2, CD3G, IL32, CD8B, | | |
| | | | CD19, CD86, AIM2, CD38, | | |
| | | | CYTIP, LOC96610, CD69, | | |
| | | | CD79A | | |
| ICS5 | Immune | mRNA | CXCL13, CLIC5, HLA-F, | 1) Mean center, | PMID: 24172169 |
| | | | TNFRSF17, XCL2 | 2) average over genes, | |
| | | | | 3) Z-score | |
| B_cells | Immune | mRNA | BLK, CD19, FCRL2, KIAA0125, | 1) Average over | PMID: 28239471 |
| | | | MS4A1, PNOC, SPIB, TCL1A, | genes, 2) mean | |
| | | | TNFRSF17 | center, 3) Z-score | |
| Dendritic_cells | Immune | mRNA | CCL13, CD209, HSD11B1 | 1) Average over | PMID: 28239471 |
| | | | | genes, 2) mean | |
| | | | | center, 3) Z-score | |
| Mast_cells | Immune | mRNA | CPA3, HDC, MS4A2, TPSAB1, | 1) Average over | PMID: 28239471 |
| | | | TPSB2 | genes, 2) mean | |
| | | | | center, 3) Z-score | |
| STAT1_sig | Immune | mRNA | TAP1, GBP1, IFIH1, PSMB9, | 1) Mean center, | PMID: 19272155 |
| | | | CXCL9, IRF1, CXCL11, CXCL10, | 2) average over | |
| | | | IDO1, STAT1 | genes, 3) Z-score | |
| Chemokine12 | Immune | mRNA | CCL2, CCL3, CCL4, CCL5, CCL8, | 1) Mean center, | PMID: 21703392 |
| | | | CCL18, CCL19, CCL21, CXCL9, | 2) average over | |
| | | | CXCL10, CXCL11, CXCL13 | genes, 3) Z-score | |
| Module3_IFN | Immune | mRNA | IFI44, IFI44L, DDX58, IFI6, | 1) Mean center, | PMID: 24516633 |
| | | | IFI27, IFIT2, IFIT1, IFIT3, | 2) take modified | |
| | | | CXCL10, MX1, OAS1, OAS2, | inner product | |
| | | | OAS3, HERC5, SAMD9, HERC6, | with centroid as | |
| | | | DDX60, RTP4, IFIH1, STAT1, | published and | |
| | | | TAP1, OASL, RSAD2, ISG15 | described below | |
| | | | | (though | |
| | | | | averaging would | |
| | | | | yield similar | |
| | | | | results), | |
| | | | | 3) Z-score | |
| Module11_Prolif | Proliferation | mRNA | CDKN3, NDC80, RNASEH2A, | 1) Mean center, | PMID: 24516633 |
| | | | CENPA, SMC2, CENPE, | 2) take modified | |
| | | | RAD51AP1, PLK4, NMU, KIF2C, | inner product | |
| | | | TMSB15A, UBE2C, CHEK1, | with centroid as | |
| | | | ZWINT, OIP5, CRABP1, ECT2, | published and | |
| | | | EIF4EBP1, EZH2, FEN1, | described below | |
| | | | HSPA4L, TPX2, FOXM1, | (though | |
| | | | NCAPH, PRAME, PDSS1, KIF4A, | averaging would | |
| | | | RAD54B, ASPM, FBXO5, | yield similar | |
| | | | ATAD2, RACGAP1, GPSM2, | results), | |
| | | | DONSON, HMMR, BIRC5, | 3) Z-score | |
| | | | KIF11, LMNB1, MAD2L1, | | |
| | | | MCM4, MCM5, MKI67, | | |
| | | | MMP1, MYBL1, MYBL2, NEK2, | | |
| | | | NUSAP1, GTSE1, GINS2, PLK1, | | |
| | | | FAM64A, ERCC6L, NCAPG2, | | |
| | | | CEP55, FANCI, HJURP, | | |
| | | | MCM10, DEPDC1, C1orf112, | | |
| | | | CENPN, PBK, KIF15, CIAPIN1, | | |
| | | | ACTR3B, GPR126, SPC25, | | |
| | | | RAD21, RFC3, RFC4, RRM2, | | |
| | | | NCAPG, STIL, SKP2, SOX11, | | |
| | | | SQLE, AURKA, TAF2, TARS, | | |
| | | | BUB1B, TK1, TMPO, TOP2A, | | |
| | | | PHLDA2, TTK, LRP8, DSCC1, | | |
| | | | MLF1IP, E2F8, SHCBP1, | | |
| | | | SLC7A5, ANP32E, KIF18A, | | |
| | | | CDC7, CDC45, RAD54L, TTF2, | | |
| | | | PIR, ACTL6A, GGH, CCNA2, | | |
| | | | CCNB1, PRC1, CCNB2, CCNE2, | | |
| | | | EXO1, AURKB, PTTG1, TRIP13, | | |
| | | | KIF23, APOBEC3B, MTFR1, | | |
| | | | ESPL1, DLGAP5, CDK1, MELK, | | |
| | | | GINS1, CDC6, CDC20, NCAPD2, | | |
| | | | KIF14 | | |
| MP_index_adj*(−1) | Proliferation | mRNA | AA834945, AI224578, | 1) MP index | PMID: 11823860 |
| | | | AI283268, ALDH4A1, AP2B1, | I(MPI) from | |
| | | | AW014921, AYTL2, BBC3, | Agendia | |
| | | | C16orf61, C20orf46, C9orf30, | (proprietary but | |
| | | | CDC42BPA, CDCA7, CENPA, | based on | |
| | | | COL4A2, DCK, DIAPH3, | publication), 2) | |
| | | | DIAPH3, DIAPH3, | adjust by | |
| | | | DKFZP686P18101, DTL, ECT2, | platform by | |
| | | | EGLN1, ESM1, EXT1, FBXO31, | adding 0.154 to | |
| | | | FGF18, FLT1, GMPS, GNAZ, | MPI from | |
| | | | GPR126, GPR180, GSTM3, | samples assayed | |
| | | | HRASLS, IGFBP5, IGFBP5, | on Agilent 44K | |
| | | | KNTC2, LGP2, LOC286052, | (GPL16233; | |
| | | | LOC643008, MCM6, MELK, | n = 333) and 0.336 | |
| | | | MMP9, MS4A7, MTDH, NMU, | to samples | |
| | | | NM_004702, NUSAP1, ORC6L, | assayed on | |
| | | | OXCT1, PALM2-AKAP2, PECI, | Agilent 32K | |
| | | | PECI, PITRM1, PQLC2, PRC1, | (GPL20078; | |
| | | | QSCN6L1, RAB6A, RFC4, | n = 654), 3) | |
| | | | RP5-860F19.3, RTN4RL1, | multiply by (−1) | |
| | | | RUNDC1, SCUBE2, | so high values | |
| | | | SLC2A14, STK32B, | indicate higher | |
| | | | TGFB3, TSPYL5, UCHL5, | risk/proliferation. | |
| | | | WISP1, ZNF533 | | |
| Basal_Index | Proliferation | mRNA | ABCC11, ACADSB, AFF3, AGF2, | Z-score | PMID: 21814749 |
| (Basal-type) | | | AR, CA12, CAPN13, CDCA7, | Basalindex values | |
| | | | CHAD, DHRS2, ESR1, FOXA1, | from BluePrint | |
| | | | FOXC1, GATA3, GREB1, | (Agendia). | |
| | | | KIAA1370, MAGED2, MLPH, | Scoring algorithm | |
| | | | MSN, MYO5C, PERLD1, PRR15, | proprietary but | |
| | | | REEP6, RTN4L1, SLC16A6, | based on nearest | |
| | | | SPEF1, TBC1D9, THSD4 | centroid method | |
| | | | | in publication | |
| ESR1_PGR_ave | ER | mRNA | ESR1, PGR | 1) Mean center, | (average of 2 |
| | | | | 2) average over | genes - |
| | | | | genes, 3) Z-score | canonical ER) |
| Luminal_Index | ER | mRNA | ABAT, ACADSB, ACBD4, ADM, | Z-score Luminal | PMID: 21814749 |
| (Luminal-type) | | | AFF3, BCL2, BECN1, BTD, | index values from | |
| | | | BTRC, CA12, CCDC74B, | BluePrint | |
| | | | CDC25B, CELSR1, CELSR2, | (Agendia). | |
| | | | CHAD, COQ7, DNALI1, ELOVL5, | Scoring algorithm | |
| | | | ESR1, GATA3, GOLSYN, GREB1, | proprietary but | |
| | | | HDAC11, HK3, HMGCL, IL6ST, | based on nearest | |
| | | | IRS1, KIAA1737, KIF20A, | centroid method | |
| | | | LILRB3, LRIG1, MYB, NAT1, | in publication | |
| | | | NPY1R, NUDT6, OCIAD1, | | |
| | | | PARD6B, PGR, PPAPDC2, | | |
| | | | PREX1, RERG, RUNDC1, | | |
| | | | S100A8, SCUBE2, SOX11, | | |
| | | | SUSD3, TAPT1, TBC1D9, | | |
| | | | TCTN1, THSD4, TMC4, | | |
| | | | TMEM101, TMSB10, TPRG1, | | |
| | | | UBXD3, DBNDD2, VAV3, XBP1 | | |
| PARPi7 | DRD | mRNA | Prediction genes: BRCA1, | 1) divide each | PMID: 22875744 |
| | | | CHEK2, MAPKAPK2, MRE11A, | PARPi-7 predictor | PMID: 28948212 |
| | | | NBN, TDG, XPA; Normalization | gene level (not | |
| | | | genes: RPL24, ABI2, GGA1, | centered) by the | |
| | | | E2F4, IPO8, CXXC1, RPS10 | geometric mean | |
| | | | | of the | |
| | | | | normalization | |
| | | | | genes, 2) log2- | |
| | | | | transform each | |
| | | | | ratio and median | |
| | | | | center, 3) | |
| | | | | calculate score as | |
| | | | | Weights*(Genes − | |
| | | | | Boundaries), using | |
| | | | | Weights = (−0.5320, 0.5806, | |
| | | | | 0.0713, −0.1396, −0.1976, | |
| | | | | −0.3937, −0.2335) | |
| | | | | and Boundaries = | |
| | | | | (−0.0153, −0.006, | |
| | | | | 0.0031, −0.0044, | |
| | | | | 0.0014, −0.0165, −0.0126), 4) | |
| | | | | standardize to | |
| | | | | sd = 1 | |
| PARPi7_plus_MP2 | DRD | mRNA | Genes in PARPi7 + Genes in | 1) PARPi7 + | PMID: 28948212 |
| | | | MP_index | MP_index_adj*(−1), | |
| | | | | 2) Z-score | |
| VCpred_TN | DRD/Immune | mRNA | CXCL13, BRCA1, APEX1, FEN1, | 1) mean center, | Exploratory - |
| | | | CD8A, SEM1, APEX2, RNMT, | 2) calculate | developed |
| | | | CCR7, H2AFX, POLD3, PRKDC, | weighted average = | from I-SPY 2 |
| | | | C1QA, CLIC5, RAD51, DDB2, | (13.60*CXCL13 − | data (VC arm) |
| | | | SPP1, OLD2 POLB, LIG1, | 6.48*BRCA1 + | as described |
| | | | GTF2H5, PMS2, LY9, SHPRH | 6.41*APEX1 + | below, and |
| | | | | 5.32*FEN1 + | validated in |
| | | | | 4.85*CD8A − | BrighTNess |
| | | | | 4.84*SEM1 + | |
| | | | | 4.78*APEX2 − | |
| | | | | 4.60*RNMT + | |
| | | | | 4.51*CCR7 + | |
| | | | | 3.99*H2AFX + | |
| | | | | 3.88*POLD3 − | |
| | | | | 3.49*PRKDC + | |
| | | | | 3.48*C1QA + | |
| | | | | 3.33*CLIC5 − | |
| | | | | 3.24*RAD51 + | |
| | | | | 3.10 *DDB2 − | |
| | | | | 2.83*SPP1 − 2.80 | |
| | | | | *POLD2 − | |
| | | | | 2.80*POLB + | |
| | | | | 2.72*LIG1 − | |
| | | | | 2.67*GTF2H5 − | |
| | | | | 2.63*PMS2 + | |
| | | | | 2.60*LY9 − | |
| | | | | 2.34*SHPRH + | |
| | | | | 6.27*ARAF), 3) Z-score | |
| HER2_Index | ERBB2 | mRNA | ERBB2, GRB7, PERLD1, SYCPB | Z-score HER2 | PMID: 21814749 |
| (HER2_type) | | | | index values from | |
| | | | | BluePrint | |
| | | | | (Agendia). | |
| | | | | Scoring algorithm | |
| | | | | proprietary but | |
| | | | | based on nearest | |
| | | | | centroid method | |
| | | | | in publication | |
| Module7_ERBB2 | ERBB2 | mRNA | ERBB2, GRB7, STARD3, PGAP3 | 1) Mean center, | PMID: 24516633 |
| | | | | 2) take modified | |
| | | | | inner product | |
| | | | | with centroid as | |
| | | | | published and | |
| | | | | described below, | |
| | | | | 3) Z-score | |
| ERBB2 Y1248 | ERBB2 | RPPA | phospho-protein ERBB2 Y1248 | Z-score values | PMID: 32914002 |
| EGFR Y1173 | ERBB2 | RPPA | phospho-protein EGFR Y1173 | Z-score values | PMID: 32914002 |
| mTOR S2448 | AKT/mTOR | RPPA | phospho-protein mTOR S2448 | Z-score values | PMID: 33083527 |
| IGF1R | AKT/mTOR | mRNA | IGF1R | Z-score values | PMID: 33083527 |
| STMN1 | AKT/mTOR | mRNA | STMN1 | Z-score values | PMID: 32914002 |
| TIE2 Y992 | Other | RPPA | phospho-protein TIE2 Y992 | Z-score values | DOI: 10.1200/ |
| | (ANG/TIE) | | | | JCO.2018.36.15_suppl.12103 |
| | | | | | DOI: 10.1158/ |
| | | | | | 1538-7445.AM2018-2611 |
| Module10_ECM | Other (ECM) | mRNA | CDH11, CDH13, LRRC17, | 1) Mean center, | PMID: 24516633 |
| | | | SPON1, POSTN, COL1A1, | 2) take modified | |
| | | | COL1A2, COL3A1, COL5A1, | inner product | |
| | | | COL5A2, COL6A1, COL6A2, | with centroid as | |
| | | | COL6A3, LRRC15, VCAN, | published and | |
| | | | PRUNE2, DPYSL3, EDNRA, FAP, | described below | |
| | | | FBN1, FGF5, NID2, FBXL7, FN1, | (though | |
| | | | ZFPM2, ANGPTL2, OLFML2B, | averaging would | |
| | | | GPR124, GAS1, DKK3, SRPX2, | yield similar | |
| | | | ITGA4, LOX, LUM, MMP2, | results), | |
| | | | MN1, NAP1L3, NID1, DDR2, | 3) Z-score | |
| | | | OMD, NOX4, PCOLCE, DACT1, | | |
| | | | PDE1C, PDGFRA, PRRX1, ASPN, | | |
| | | | RCN3, SLIT3, SPARC, SPOCK1, | | |
| | | | ZEB1, TNFAIP6, SCG2, | | |
| | | | ADAM12, JAM3, MSC, ITGBL1 | | |
| RPL24 | Other | mRNA | RPL24 | Z-score values | PMID: 24970821 |
| LYMPHS_PCA | Other | mRNA | UQCRB, SESTD1, QTRT1, TIPIN, | 1) Mean center, | PMID: 16704732 |
| | | | REL, STXBP2, HSBP1, COX6C, | 2) calculate PCA | |
| | | | RPL11, MECOM, ANKRD28, | (d.pca <− | |
| | | | JUN, ZC3H15, RPL23, | prcomp(~., data = | |
| | | | RPS6KA2, EEF2, TMA7, RPS6, | data.frame(dat), | |
| | | | RPL27, RPS21, COX7B, | center = F, scale = F, | |
| | | | PRRC2B, CYP17A1, NSUN4, | na.action = | |
| | | | TOMM34, MINOS1, | na.omit)$rotation | |
| | | | STAMBPL1, FGF9, ATF4, | [,1]), 2) Z-score, | |
| | | | RPL35, RPL31, RPS24, | 3) multiply by (−1) | |
| | | | DCLRE1C, C5orf49, FAM162A, | if cor(d.pca, | |
| | | | ITGB2, SLC19A1, RPL32, TPP2, | mean(dat)) <− | |
| | | | MALAT1, LSM3, TSSC1, | 0.25 | |
| | | | ATXN2L, SERPINB6, TPI1 | | |

TABLE 2
Columns A-I

| | All.adj.HRHE | | All.adj.HRHE | Ctr_All.adj. |
|---|---|---|---|---|
| | R2Arm: | All.adj.HRHE | R2Arm: BH | HRHER2: |
| | OR/1SD | R2Arm: LR p | LR p | OR/1SD |
| ICSS_score | 1.85 | 4.02E−15 | 1.52E−12 | 1.82 |
| Chemokine12_score | 1.93 | 5.13E−18 | 2.91E−13 | 2.02 |
| Module5_TcellBcell_score | 1.81 | 5.71E−14 | 1.30E−11 | 1.67 |
| STAT1_sig | 1.78 | 5.39E−13 | 1.02E−10 | 1.7 |
| Module3_IFN_score | 1.2 | 0.013 | 0.0699 | 1.09 |
| Dendritic_cells | 1.59 | 1.69E−09 | 1.37E−07 | 1.2 |
| B_cells | 1.58 | 1.10E−09 | 1.13E−07 | 1.31 |
| Mast_cells | 0.721 | 0.000212 | 0.00311 | 0.8 |
| Module11_Prolif_score | 1.43 | 2.62E−05 | 0.000632 | 1.53 |
| MP_ index_adj*(−1) | 1.91 | 2.18E−10 | 3.53E−08 | 1.59 |
| Basal_Index | 1.6 | 4.55E−05 | 0.00101 | 1.1 |
| PARPi7_score | 1.23 | 0.00795 | 0.0495 | 1.09 |
| PARPi7_plus_MP2 | 1.38 | 0.000123 | 0.00225 | 1.16 |
| VCpred_TN | 1.91 | 1.57E−16 | 1.78E−13 | 1.95 |
| STMN1_dat | 1.45 | 9.43E−06 | 0.000297 | 1.14 |
| HER2_Index | 1.73 | 2.14E−05 | 0.000539 | 1.14 |
| Mod7_ERBB2 | 1.72 | 3.01E−05 | 0.000697 | 1.12 |
| ERBB2.Y1248 | 1.68 | 3.79E−08 | 0.000142 | 1.7 |
| EGFR.Y1173 | 1.64 | 1.90E−06 | 8.29E−05 | 2.04 |
| mTOR.S2448 | 1.09 | 0.335 | 0.57 | 1.05 |
| IGF1R_dat | 0.673 | 1.71E−05 | 0.000462 | 0.505 |
| TIE2.Y992 | 1.08 | 0.431 | 0.655 | 1.17 |
| Mod10_ECM | 0.884 | 0.104 | 0.286 | 0.946 |
| RPL24_dat | 0.986 | 0.846 | 0.94 | 1.14 |
| LYMPHS_PCA_16704732 | 0.791 | 0.00327 | 0.0254 | 1.03 |
| Luminal_Index | 0.417 | 1.05E−14 | 2.98E−12 | 0.463 |
| ER_PGR_avg | 0.506 | 8.41E−10 | 1.06E−07 | 0.592 |
| | | | N_All.adj.HR | |
| | Ctr_All.adj. | Ctr_Allad.HR | HER2: | N_All.adj.HRH |
| | HRHER2: LR p | HER2: BH LR p | OR/1SD | ER2: LR p |
| ICSS_score | 0.00142 | 0.014 | 1.43 | 0.0802 |
| Chemokine12_score | 0.000304 | 0.00406 | 1.73 | 0.0102 |
| Module5_TcellBcell_score | 0.00653 | 0.0431 | 1.59 | 0.0227 |
| STAT1_sig | 0.00449 | 0.0328 | 1.54 | 0.0402 |
| Module3_IFN_score | 0.64 | 0.813 | 1.05 | 0.787 |
| Dendritic_cells | 0.327 | 0.565 | 1.84 | 0.0098 |
| B_cells | 0.128 | 0.329 | 1.59 | 0.0274 |
| Mast_cells | 0.273 | 0.505 | 1.01 | 0.96 |
| Module11_Prolif_score | 0.0407 | 0.146 | 1.45 | 0.159 |
| MP_ index_adj*(−1) | 0.0495 | 0.171 | 2.44 | 0.00386 |
| Basal_Index | 0.728 | 0.867 | 2.05 | 0.0374 |
| PARPi7_score | 0.61 | 0.793 | 1.21 | 0.425 |
| PARPi7_plus_MP2 | 0.409 | 0.636 | 1.49 | 0.137 |
| VCpred_TN | 0.000217 | 0.00311 | 1.41 | 0.0771 |
| STMN1_dat | 0.529 | 0.732 | 1.65 | 0.0554 |
| HER2_Index | 0.678 | 0.841 | 2.07 | 0.0227 |
| Mod7_ERBB2 | 0.735 | 0.867 | 2.41 | 0.00406 |
| ERBB2.Y1248 | 0.111 | 0.296 | 1.73 | 0.00484 |
| EGFR.Y1173 | 0.0537 | 0.18 | 1.58 | 0.0119 |
| mTOR.S2448 | 0.764 | 0.885 | 1.24 | 0.337 |
| IGF1R_dat | 0.00249 | 0.0206 | 0.751 | 0.338 |
| TIE2.Y992 | 0.526 | 0.73 | 0.888 | 0.658 |
| Mod10_ECM | 0.777 | 0.896 | 0.838 | 0.393 |
| RPL24_dat | 0.42 | 0.646 | 1.07 | 0.751 |
| LYMPHS_PCA_16704732 | 0.889 | 0.967 | 0.639 | 0.1 |
| Luminal_Index | 0.00243 | 0.0204 | 0.273 | 0.000792 |
| ER_PGR_avg | 0.0265 | 0.11 | 0.434 | 0.0205 |

Columns J-S

| | N_All.adj.HR | MK2206_All. | MK2206_All. | MK2206_All. | AMG386_All. |
|---|---|---|---|---|---|
| | HER2: BH | adj.HRHER2: | adj.HRHER2: | adj.HRHER2: | adj.HRHER2: |
| | LR p | OR/1SD | LR p | BH LR p | OR/1SD |
| ICSS_score | 0.24 | 1.76 | 0.0194 | 0.0902 | 2.36 |
| Chemokine12_score | 0.0593 | 1.6 | 0.0717 | 0.223 | 2.56 |
| Module5_TcellBcell_score | 0.101 | 1.55 | 0.0782 | 0.236 | 2.44 |
| STAT1_sig | 0.146 | 1.29 | 0.327 | 0.565 | 2.44 |
| Module3_IFN_score | 0.902 | 1.03 | 0.924 | 0.992 | 1.23 |
| Dendritic_cells | 0.0579 | 1.28 | 0.297 | 0.532 | 2.2 |
| B_cells | 0.113 | 1.73 | 0.0191 | 0.0895 | 1.64 |
| Mast_cells | 1 | 0.862 | 0.566 | 0.764 | 0.743 |
| Module11_Prolif_score | 0.374 | 1.14 | 0.58 | 0.777 | 1.08 |
| MP_ index_adj*(−1) | 0.0292 | 1.19 | 0.549 | 0.752 | 1.48 |
| Basal_Index | 0.14 | 0.942 | 0.878 | 0.96 | 1.73 |
| PARPi7_score | 0.65 | 0.809 | 0.394 | 0.622 | 1.63 |
| PARPi7_plus_MP2 | 0.343 | 0.843 | 0.511 | 0.718 | 1.75 |
| VCpred_TN | 0.235 | 1.52 | 0.0919 | 0.262 | 2.63 |
| STMN1_dat | 0.184 | 1.3 | 0.221 | 0.446 | 1.23 |
| HER2_Index | 0.101 | 0.773 | 0.565 | 0.764 | 1.44 |
| Mod7_ERBB2 | 0.0303 | 1.42 | 0.443 | 0.661 | 0.899 |
| ERBB2.Y1248 | 0.0347 | 1.46 | 0.186 | 0.402 | 1.04 |
| EGFR.Y1173 | 0.0652 | 1.57 | 0.0651 | 0.208 | 0.787 |
| mTOR.S2448 | 0.57 | 1.29 | 0.288 | 0.519 | 0.896 |
| IGF1R_dat | 0.57 | 0.89 | 0.705 | 0.858 | 0.506 |
| TIE2.Y992 | 0.825 | 0.974 | 0.934 | 0.995 | 1.13 |
| Mod10_ECM | 0.622 | 0.771 | 0.271 | 0.504 | 1.19 |
| RPL24_dat | 0.879 | 1.75 | 0.0494 | 0.171 | 0.998 |
| LYMPHS_PCA_16704732 | 0.278 | 1.8 | 0.0316 | 0.124 | 0.703 |
| Luminal_Index | 0.00895 | 1.1 | 0.808 | 0.915 | 0.399 |
| ER_PGR_avg | 0.0926 | 0.994 | 0.986 | 1 | 0.355 |
| | AMG386_All. | AMG386_All. | VC_All.adj.HR | | |
| | adj.HRHER2: | adj.HRHER2: | HER2: | VC_All.adj.HR | VC_All.adj.HR |
| | LR p | BH LR p | OR/1SD | HER2: LR p | HER2: BH LR p |
| ICSS_score | 0.000142 | 0.00237 | 1.89 | 0.0374 | 0.14 |
| Chemokine12_score | 0.000141 | 0.00237 | 1.99 | 0.0257 | 0.108 |
| Module5_TcellBcell_score | 0.000103 | 0.00195 | 1.96 | 0.0254 | 0.107 |
| STAT1_sig | 0.000265 | 0.00366 | 2.04 | 0.0126 | 0.0684 |
| Module3_IFN_score | 0.321 | 0.56 | 1.47 | 0.201 | 0.417 |
| Dendritic_cells | 0.00014 | 0.00237 | 2.2 | 0.0103 | 0.0596 |
| B_cells | 0.0133 | 0.0707 | 1.56 | 0.141 | 0.349 |
| Mast_cells | 0.193 | 0.41 | 0.914 | 0.763 | 0.885 |
| Module11_Prolif_score | 0.745 | 0.876 | 2.8 | 0.0147 | 0.0758 |
| MP_ index_adj*(−1) | 0.154 | 0.369 | 4.46 | 0.00316 | 0.0251 |
| Basal_Index | 0.0778 | 0.236 | 5.67 | 0.000471 | 0.00593 |
| PARPi7_score | 0.0312 | 0.124 | 4.07 | 0.000156 | 0.00251 |
| PARPi7_plus_MP2 | 0.0197 | 0.0908 | 5.63 | 2.72E−05 | 0.000643 |
| VCpred_TN | 7.79E−05 | 0.00161 | 4.38 | 1.43E−05 | 0.000405 |
| STMN1_dat | 0.363 | 0.591 | 2.5 | 0.00955 | 0.0568 |
| HER2_Index | 0.437 | 0.659 | 0.584 | 0.48 | 0.694 |
| Mod7_ERBB2 | 0.792 | 0.904 | 0.666 | 0.709 | 0.859 |
| ERBB2.Y1248 | 0.929 | 0.994 | 0.521 | 0.518 | 0.723 |
| EGFR.Y1173 | 0.659 | 0.825 | 0.486 | 0.478 | 0.693 |
| mTOR.S2448 | 0.62 | 0.8 | 1.07 | 0.832 | 0.927 |
| IGF1R_dat | 0.00783 | 0.0491 | 0.703 | 0.34 | 0.571 |
| TIE2.Y992 | 0.499 | 0.708 | 1.12 | 0.599 | 0.788 |
| Mod10_ECM | 0.364 | 0.591 | 1.31 | 0.435 | 0.657 |
| RPL24_dat | 0.992 | 1 | 0.465 | 0.0133 | 0.0707 |
| LYMPHS_PCA_16704732 | 0.162 | 0.376 | 0.0764 | 3.11E−07 | 1.60E−05 |
| Luminal_Index | 0.00333 | 0.0257 | 0.105 | 0.000102 | 0.00195 |
| ER_PGR_avg | 0.000654 | 0.00789 | 0.403 | 0.0406 | 0.146 |

Columns T-AC

| | Pembro_All. | Pembro_All. | Pembro_All. | Ganitumab_All. | Ganitumab_All. |
|---|---|---|---|---|---|
| | adj.HRHER2: | adj.HRHER2: | adj.HRHER2: | adj.HRHER2: | adj.HRHER2: |
| | OR/1SD | LR p | BH LR p | OR/1SD | LR p |
| ICSS_score | 2.55 | 0.000536 | 0.00668 | 2.24 | 0.00141 |
| Chemokine12_score | 3.42 | 0.000117 | 0.00218 | 1.71 | 0.0245 |
| Module5_TcellBcell_score | 3.22 | 0.000177 | 0.00271 | 1.93 | 0.00632 |
| STAT1_sig | 3.78 | 9.05E−05 | 0.0018 | 1.74 | 0.0161 |
| Module3_IFN_score | 1.63 | 0.075 | 0.23 | 1.32 | 0.259 |
| Dendritic_cells | 3.58 | 8.71E−05 | 0.00176 | 1.59 | 0.0517 |
| B_cells | 2.25 | 0.00132 | 0.0135 | 2.26 | 0.00206 |
| Mast_cells | 0.459 | 0.0105 | 0.0601 | 0.598 | 0.116 |
| Module11_Prolif_score | 1.42 | 0.192 | 0.409 | 1.75 | 0.0347 |
| MP_ index_adj*(−1) | 2.06 | 0.0315 | 0.124 | 2.16 | 0.0197 |
| Basal_Index | 3.01 | 0.00264 | 0.0214 | 1.74 | 0.18 |
| PARPi7_score | 1.29 | 0.332 | 0.569 | 1.36 | 0.196 |
| PARPi7_plus_MP2 | 1.46 | 0.178 | 0.396 | 1.53 | 0.0929 |
| VCpred_TN | 2.32 | 0.00189 | 0.017 | 2.16 | 0.00127 |
| STMN1_dat | 1.8 | 0.0651 | 0.208 | 1.73 | 0.0328 |
| HER2_Index | 0.0654 | 0.274 | 0.505 | 1.04 | 0.968 |
| Mod7_ERBB2 | 0.682 | 0.6 | 0.788 | 0.585 | 0.445 |
| ERBB2.Y1248 | 0.455 | 0.777 | 0.896 | NA | NA |
| EGFR.Y1173 | 0.83 | 0.943 | 0.999 | NA | NA |
| mTOR.S2448 | 0.756 | 0.382 | 0.614 | NA | NA |
| IGF1R_dat | 0.556 | 0.0681 | 0.215 | 0.981 | 0.948 |
| TIE2.Y992 | NA | NA | NA | NA | NA |
| Mod10_ECM | 0.614 | 0.0623 | 0.202 | 0.575 | 0.0238 |
| RPL24_dat | 0.769 | 0.262 | 0.492 | 0.911 | 0.74 |
| LYMPHS_PCA_16704732 | 0.733 | 0.112 | 0.297 | 0.738 | 0.286 |
| Luminal_Index | 0.376 | 0.01 | 0.0588 | 0.554 | 0.0921 |
| ER_PGR_avg | 0.311 | 0.0024 | 0.0203 | 0.793 | 0.441 |
| | Ganitumab_All. | Ganetespib_ | Ganetespib_ | Ganetespib_ | Pertuzumab_ |
| | adj.HRHER2: | All.adj.HRHE | All.adj.HRHE | All.adj.HRHE | All.adj.HRH |
| | BH LR p | R2: OR/1SD | R2: LR p | R2: BH LR p | ER2: OR/1SD |
| ICSS_score | 0.014 | 1.65 | 0.0664 | 0.211 | 1.9 |
| Chemokine12_score | 0.105 | 1.56 | 0.0869 | 0.252 | 2.66 |
| Module5_TcellBcell_score | 0.0419 | 1.5 | 0.128 | 0.329 | 1.57 |
| STAT1_sig | 0.0815 | 1.57 | 0.0703 | 0.221 | 1.56 |
| Module3_IFN_score | 0.489 | 1.28 | 0.258 | 0.488 | 1.02 |
| Dendritic_cells | 0.176 | 1.06 | 0.82 | 0.923 | 1.43 |
| B_cells | 0.0181 | 1.2 | 0.498 | 0.708 | 1.78 |
| Mast_cells | 0.306 | 0.481 | 0.0402 | 0.146 | 0.548 |
| Module11_Prolif_score | 0.132 | 0.989 | 0.969 | 1 | 2.84 |
| MP_ index_adj*(−1) | 0.0908 | 1.65 | 0.185 | 0.402 | 6.38 |
| Basal_Index | 0.397 | 1.31 | 0.531 | 0.718 | 2.47 |
| PARPi7_score | 0.411 | 1.02 | 0.947 | 1 | 0.617 |
| PARPi7_plus_MP2 | 0.263 | 1.09 | 0.761 | 0.884 | 1.01 |
| VCpred_TN | 0.0132 | 1.78 | 0.0342 | 0.131 | 1.67 |
| STMN1_dat | 0.128 | 1.35 | 0.316 | 0.555 | 2.24 |
| HER2_Index | 1 | 8.76 | 0.237 | 0.463 | 2.02 |
| Mod7_ERBB2 | 0.661 | 1.23 | 0.781 | 0.898 | 2.3 |
| ERBB2.Y1248 | NA | NA | NA | NA | 2.75 |
| EGFR.Y1173 | NA | NA | NA | NA | 1.85 |
| mTOR.S2448 | NA | NA | NA | NA | 1.97 |
| IGF1R_dat | 1 | 1.19 | 0.494 | 0.705 | 0.441 |
| TIE2.Y992 | NA | NA | NA | NA | NA |
| Mod10_ECM | 0.104 | 1.02 | 0.926 | 0.993 | 0.946 |
| RPL24_dat | 0.873 | 0.68 | 0.092 | 0.262 | 1.2 |
| LYMPHS_PCA_16704732 | 0.518 | 0.538 | 0.0201 | 0.0919 | 1.17 |
| Luminal_Index | 0.262 | 0.551 | 0.132 | 0.334 | 0.246 |
| ER_PGR_avg | 0.66 | 0.537 | 0.159 | 0.374 | 0.124 |

Columns AD-AM

| | Pertuzumab_ | Pertuzumab_ | TDM1/P_All. | TDM1/P_All. | TDM1/P_All. |
|---|---|---|---|---|---|
| | All.adj.HRH | All.adj.HRH | adj.HRHER2: | adj.HRHER2: | adj.HRHER2: |
| | ER2: LR p | ER2: BH LR p | OR/1SD | LR p | BH LR p |
| ICSS_score | 0.0755 | 0.231 | 1.59 | 0.159 | 0.374 |
| Chemokine12_score | 0.131 | 0.332 | 2.15 | 0.0388 | 0.143 |
| Module5_TcellBcell_score | 0.168 | 0.387 | 1.64 | 0.16 | 0.374 |
| STAT1_sig | 0.175 | 0.396 | 1.82 | 0.175 | 0.396 |
| Module3_IFN_score | 0.955 | 1 | 1.22 | 0.538 | 0.741 |
| Dendritic_cells | 0.351 | 0.582 | 1.52 | 0.186 | 0.402 |
| B_cells | 0.122 | 0.318 | 1.5 | 0.175 | 0.396 |
| Mast_cells | 0.176 | 0.396 | 0.562 | 0.137 | 0.343 |
| Module11_Prolif_score | 0.0254 | 0.107 | 2.42 | 0.0293 | 0.119 |
| MP_ index_adj*(−1) | 7.00E−04 | 0.00834 | 3.39 | 0.0048 | 0.0347 |
| Basal_Index | 0.198 | 0.414 | 0.611 | 0.387 | 0.616 |
| PARPi7_score | 0.338 | 0.57 | 1.6 | 0.277 | 0.508 |
| PARPi7_plus_MP2 | 0.981 | 3. | 2.29 | 0.0715 | 0.223 |
| VCpred_TN | 0.17 | 0.389 | 1.27 | 0.482 | 0.696 |
| STMN1_dat | 0.0584 | 0.192 | 1.32 | 0.466 | 0.679 |
| HER2_Index | 0.0205 | 0.0926 | 3.9 | 2.92E−06 | 0.000123 |
| Mod7_ERBB2 | 0.0111 | 0.0617 | 5.07 | 5.71E−06 | 0.00019 |
| ERBB2.Y1248 | 0.0212 | 0.0954 | 6.07 | 0.00016 | 0.00252 |
| EGFR.Y1173 | 0.0672 | 0.213 | 24.3 | 3.67E−06 | 0.000142 |
| mTOR.S2448 | 0.212 | 0.432 | 1.34 | 0.401 | 0.628 |
| IGF1R_dat | 0.0432 | 0.153 | 0.339 | 0.0164 | 0.0819 |
| TIE2.Y992 | NA | NA | NA | NA | NA |
| Mod10_ECM | 0.876 | 0.96 | 0.808 | 0.523 | 0.727 |
| RPL24_dat | 0.692 | 0.849 | 1.69 | 0.194 | 0.41 |
| LYMPHS_PCA_16704732 | 0.73 | 0.867 | 0.73 | 0.438 | 0.659 |
| Luminal_Index | 0.00123 | 0.0129 | 0.123 | 0.000157 | 0.00251 |
| ER_PGR_avg | 0.000939 | 0.0101 | 0.208 | 0.0109 | 0.0612 |
| | HR+HER2−. | | HR+HER2−. | | |
| | adj.Tx: | HR+HER2−. | adj.Tx: BH | Ctr_HR+HER2−: | Ctr_HR+HER2−: |
| | OR/1SD | adj.Tx: LR p | LR p | OR/1SD | LR p |
| ICSS_score | 2.43 | 1.27E−09 | 1.20E−07 | 1.92 | 0.026 |
| Chemokine12_score | 2.5 | 1.06E−09 | 1.13E−07 | 2.54 | 0.00154 |
| Module5_TcellBcell_score | 2.37 | 3.64E−09 | 2.75E−07 | 1.89 | 0.0268 |
| STAT1_sig | 2.4 | 1.04E−08 | 6.55E−07 | 2.2 | 0.00908 |
| Module3_IFN_score | 1.12 | 0.42 | 0.646 | 0.794 | 0.453 |
| Dendritic_cells | 1.7 | 0.000173 | 0.00269 | 1.33 | 0.313 |
| B_cells | 1.92 | 3.89E−06 | 0.000142 | 1.35 | 0.343 |
| Mast_cells | 0.5 | 4.00E−06 | 0.000142 | 0.539 | 0.0404 |
| Module11_Prolif_score | 1.76 | 0.000139 | 0.00237 | 1.93 | 0.0329 |
| MP_ index_adj*(−1) | 2.13 | 8.50E−07 | 4.02E−05 | 2.33 | 0.00931 |
| Basal_Index | 2.13 | 7.41E−07 | 3.65E−05 | 1.81 | 0.0523 |
| PARPi7_score | 1.73 | 0.000939 | 0.0101 | 1.28 | 0.431 |
| PARPi7_plus_MP2 | 2.03 | 1.96E−05 | 0.000505 | 1.51 | 0.198 |
| VCpred_TN | 2.47 | 4.78E−10 | 6.78E−08 | 2.16 | 0.00457 |
| STMN1_dat | 1.77 | 0.000136 | 0.00237 | 1.48 | 0.207 |
| HER2_Index | 0.516 | 0.273 | 0.505 | 0.00211 | 0.0217 |
| Mod7_ERBB2 | 0.304 | 0.00216 | 0.0186 | 0.648 | 0.523 |
| ERBB2.Y1248 | 0.482 | 0.469 | 0.683 | 17.1 | 0.088 |
| EGFR.Y1173 | 1 | 0.996 | 1 | 9.24 | 0.0816 |
| mTOR.S2448 | 0.997 | 0.982 | 1 | 0.867 | 0.565 |
| IGF1R_dat | 0.577 | 0.000217 | 0.00311 | 0.445 | 0.0119 |
| TIE2.Y992 | 1.32 | 0.235 | 0.462 | 2.07 | 0.117 |
| Mod10_ECM | 0.798 | 0.121 | 0.317 | 0.579 | 0.112 |
| RPL24_dat | 1.04 | 0.802 | 0.914 | 1.25 | 0.478 |
| LYMPHS_PCA_16704732 | 0.644 | 0.00165 | 0.0156 | 0.888 | 0.665 |
| Luminal_Index | 0.435 | 9.32E−09 | 6.22E−07 | 0.548 | 0.0279 |
| ER_PGR_avg | 0.426 | 1.91E−08 | 1.14E:06 | 0.615 | 0.0893 |

Columns AN-AW

| | Ctr_HR+ | | | | MK2206_HR+ |
|---|---|---|---|---|---|
| | HER2−: | N_HR+HER2−: | N_HR+HER2−: | N_HR+HER2−: | HER2−: |
| | BH LR p | OR/1SD | LR p | BH LR p | OR/1SD |
| ICSS_score | 0.109 | 1.34 | 0.668 | 0.832 | 1.89 |
| Chemokine12_score | 0.0147 | 2.38 | 0.298 | 0.533 | 1.48 |
| Module5_TcellBcell_score | 0.111 | 2.02 | 0.344 | 0.573 | 1.78 |
| STAT1_sig | 0.0554 | 1.89 | 0.377 | 0.608 | 1.27 |
| Module3_IFN_score | 0.668 | 0.932 | 0.92 | 0.99 | 1.07 |
| Dendritic_cells | 0.551 | 2.68 | 0.176 | 0.396 | 1.07 |
| B_cells | 0.573 | 1.18 | 0.785 | 0.902 | 2.19 |
| Mast_cells | 0.146 | 0.643 | 0.548 | 0.752 | 0.797 |
| Module11_Prolif_score | 0.128 | 1.38 | 0.691 | 0.849 | 0.926 |
| MP_ index_adj*(−1) | 0.0562 | 31.9 | 0.0166 | 0.0822 | 0.891 |
| Basal_Index | 0.177 | 9.35 | 0.0445 | 0.156 | 0.851 |
| PARPi7_score | 0.655 | 1.52 | 0.479 | 0.694 | 1.27 |
| PARPi7_plus_MP2 | 0.414 | 2.2 | 0.218 | 0.442 | 1.16 |
| VCpred_TN | 0.0332 | 1.41 | 0.665 | 0.83 | 1.92 |
| STMN1_dat | 0.425 | 3.41 | 0.17 | 0.389 | 0.976 |
| HER2_Index | 0.0969 | 0.159 | 0.729 | 0.867 | 0.0249 |
| Mod7_ERBB2 | 0.727 | 0.312 | 0.614 | 0.796 | 0.55 |
| ERBB2.Y1248 | 0.254 | <0.01 | 0.373 | 0.603 | 0.0192 |
| EGFR.Y1173 | 0.242 | 0.451 | 0.906 | 0.978 | 0.158 |
| mTOR.S2448 | 0.764 | 1.03 | 0.954 | 1 | 1.81 |
| IGF1R_dat | 0.0652 | 0.46 | 0.271 | 0.504 | 0.709 |
| TIE2.Y992 | 0.308 | 1.75 | 0.621 | 0.8 | 1.23 |
| Mod10_ECM | 0.297 | 1.45 | 0.621 | 0.8 | 0.576 |
| RPL24_dat | 0.693 | 0.37 | 0.158 | 0.374 | 3.71 |
| LYMPHS_PCA_16704732 | 0.83 | <0.01 | 0.00087 | 0.00967 | 2.44 |
| Luminal_Index | 0.114 | <0.01 | 0.00176 | 0.0165 | 0.83 |
| ER_PGR_avg | 0.257 | 0.219 | 0.0574 | 0.189 | 0.838 |
| | | MK2206_HR+ | AMG386_HR+ | | AMG386_HR+ |
| | MK2206_HR+ | HER2−: BH | HER2−: | AMG386_HR+ | HER2−: BH |
| | HER2−: LR p | LR p | OR/1SD | HER2−: LR p | LR p |
| ICSS_score | 0.124 | 0.323 | 4.59 | 0.000233 | 0.0033 |
| Chemokine12_score | 0.332 | 0.569 | 3.6 | 0.00216 | 0.0186 |
| Module5_TcellBcell_score | 0.178 | 0.396 | 3.38 | 0.00192 | 0.017 |
| STAT1_sig | 0.591 | 0.783 | 2.86 | 0.0134 | 0.0707 |
| Module3_IFN_score | 0.903 | 0.976 | 0.955 | 0.878 | 0.96 |
| Dendritic_cells | 0.887 | 0.966 | 2.15 | 0.017 | 0.0838 |
| B_cells | 0.0979 | 0.274 | 2.13 | 0.0148 | 0.0759 |
| Mast_cells | 0.719 | 0.867 | 0.507 | 0.0473 | 0.166 |
| Module11_Prolif_score | 0.859 | 0.949 | 1.68 | 0.204 | 0.422 |
| MP_ index_adj*(−1) | 0.808 | 0.915 | 3.32 | 0.00696 | 0.0451 |
| Basal_Index | 0.764 | 0.885 | 2.76 | 0.0134 | 0.0707 |
| PARPi7_score | 0.696 | 0.853 | 1.48 | 0.445 | 0.661 |
| PARPi7_plus_MP2 | 0.8 | 0.913 | 2.27 | 0.127 | 0.328 |
| VCpred_TN | 0.178 | 0.396 | 3.59 | 0.0019 | 0.017 |
| STMN1_dat | 0.958 | 1 | 1.81 | 0.126 | 0.327 |
| HER2_Index | 0.205 | 0.423 | 1.08 | 0.956 | 1 |
| Mod7_ERBB2 | 0.678 | 0.841 | 0.133 | 0.0406 | 0.146 |
| ERBB2.Y1248 | 0.511 | 0.718 | <0.01 | 0.0172 | 0.0844 |
| EGFR.Y1173 | 0.652 | 0.822 | <0.01 | 0.418 | 0.646 |
| mTOR.S2448 | 0.166 | 0.384 | 0.719 | 0.489 | 0.699 |
| IGF1R_dat | 0.511 | 0.718 | 0.226 | 0.000377 | 0.00491 |
| TIE2.Y992 | 0.686 | 0.846 | 1.22 | 0.591 | 0.783 |
| Mod10_ECM | 0.254 | 0.486 | 0.939 | 0.863 | 0.951 |
| RPL24_dat | 0.0178 | 0.0863 | 1.75 | 0.165 | 0.383 |
| LYMPHS_PCA_16704732 | 0.111 | 0.296 | 0.373 | 0.0604 | 0.197 |
| Luminal_Index | 0.701 | 0.855 | 0.261 | 0.000778 | 0.00891 |
| ER_PGR_avg | 0.68 | 0.841 | 0.114 | 1.25E−05 | 0.000363 |

Columns AX-BG

| | | | | Pembro_HR+ | |
|---|---|---|---|---|---|
| | VC_HR+HER2−: | VC_HR+HER2−: | VC_HR+HER2−: | HER2−: | Pembro_HR+ |
| | OR/1SD | LR p | BH LR p | OR/1SD | HER2−: LR p |
| ICSS_score | 1.37 | 0.533 | 0.736 | 2.52 | 0.0187 |
| Chemokine12_score | 2.18 | 0.162 | 0.376 | 2.53 | 0.0214 |
| Module5_TcellBcell_score | 2.15 | 0.14 | 0.348 | 2.58 | 0.0184 |
| STAT1_sig | 3.48 | 0.0306 | 0.122 | 2.64 | 0.0239 |
| Module3_IFN_score | 2.11 | 0.23 | 0.457 | 1.25 | 0.623 |
| Dendritic_cells | 1.67 | 0.324 | 0.564 | 2.88 | 0.037 |
| B_cells | 1.07 | 0.909 | 0.979 | 2.64 | 0.00878 |
| Mast_cells | 0.541 | 0.233 | 0.46 | 0.358 | 0.00552 |
| Module11_Prolif_score | 17.4 | 0.000449 | 0.00572 | 1.69 | 0.152 |
| MP_ index_adj*(−1) | 7.54 | 0.00418 | 0.031 | 1.98 | 0.0645 |
| Basal_Index | 11.8 | 0.000215 | 0.00311 | 2.83 | 0.00956 |
| PARPi7_score | 9.96 | 0.00589 | 0.0397 | 2.81 | 0.0251 |
| PARPi7_plus_MP2 | 22.3 | 0.000797 | 0.00895 | 3.29 | 0.0107 |
| VCpred_TN | 1.79 | 0.304 | 0.539 | 2.37 | 0.0245 |
| STMN1_dat | 15.4 | 0.00134 | 0.0136 | 2.22 | 0.0736 |
| HER2_Index | 1.12 | 0.927 | 0.994 | <0.01 | 0.108 |
| Mod7_ERBB2 | 0.0776 | 0.201 | 0.417 | 0.128 | 0.0534 |
| ERBB2.Y1248 | <0.01 | 0.0385 | 0.143 | <0.01 | 0.386 |
| EGFR.Y1173 | 0.0428 | 0.353 | 0.584 | 0.0534 | 0.653 |
| mTOR.S2448 | 2.77 | 0.0379 | 0.141 | 0.551 | 0.15 |
| IGF1R_dat | 0.461 | 0.19 | 0.407 | 0.593 | 0.178 |
| TIE2.Y992 | 0.425 | 0.381 | 0.614 | NA | NA |
| Mod10_ECM | 2.08 | 0.243 | 0.473 | 0.796 | 0.488 |
| RPL24_dat | 0.769 | 0.635 | 0.808 | 0.797 | 0.455 |
| LYMPHS_PCA_16704732 | 0.0515 | 0.000402 | 0.00518 | 0.646 | 0.0832 |
| Luminal_Index | <0.01 | 2.62E−05 | 0.000632 | 0.405 | 0.0191 |
| ER_PGR_avg | <0.01 | 0.000302 | 0.00406 | 0.278 | 0.00182 |
| | Pembro HR+ | Ganitumab_ | Ganitumab_ | Ganitumab_ | Ganetespib_ |
| | HER2−: BH | HR+HER2−: | HR+HER2−: | HR+HER2−: | HR+HER2−: |
| | LR p | OR/1SD | LR p | BH LR p | OR/1SD |
| ICSS_score | 0.0887 | 3.56 | 0.0014 | 0.014 | 4.41 |
| Chemokine12_score | 0.0959 | 2.55 | 0.0237 | 0.104 | 3.18 |
| Module5_TcellBcell_score | 0.088 | 2.95 | 0.00488 | 0.0348 | 2.95 |
| STAT1_sig | 0.104 | 2.65 | 0.0181 | 0.087 | 3.49 |
| Module3_IFN_score | 0.801 | 1.3 | 0.518 | 0.723 | 2.1 |
| Dendritic_cells | 0.14 | 1.81 | 0.11 | 0.296 | 1.67 |
| B_cells | 0.0538 | 3.83 | 0.000706 | 0.00834 | 1.32 |
| Mast_cells | 0.0377 | 0.587 | 0.216 | 0.439 | 0.347 |
| Module11_Prolif_score | 0.367 | 1.82 | 0.131 | 0.332 | 1.45 |
| MP_ index_adj*(−1) | 0.208 | 2.15 | 0.0516 | 0.176 | 1.3 |
| Basal_Index | 0.0568 | 2.27 | 0.0648 | 0.208 | 1.08 |
| PARPi7_score | 0.107 | 1.79 | 0.16 | 0.374 | 1.6 |
| PARPi7_plus_MP2 | 0.0604 | 1.96 | 0.1 | 0.278 | 1.63 |
| VCpred_TN | 0.105 | 3.1 | 0.00234 | 0.02 | 3.64 |
| STMN1_dat | 0.227 | 1.72 | 0.131 | 0.332 | 1.68 |
| HER2_Index | 0.294 | 1.54 | 0.659 | 0.825 | 2.57 |
| Mod7_ERBB2 | 0.18 | 0.363 | 0.333 | 0.569 | 0.266 |
| ERBB2.Y1248 | 0.616 | NA | NA | NA | NA |
| EGFR.Y1173 | 0.823 | NA | NA | NA | NA |
| mTOR.S2448 | 0.365 | NA | NA | NA | NA |
| IGF1R_dat | 0.396 | 1.74 | 0.162 | 0.376 | 0.734 |
| TIE2.Y992 | NA | NA | NA | NA | NA |
| Mod10_ECM | 0.699 | 0.677 | 0.348 | 0.579 | 0.827 |
| RPL24_dat | 0.67 | 0.583 | 0.179 | 0.396 | 1.08 |
| LYMPHS_PCA_16704732 | 0.245 | 0.557 | 0.186 | 0.402 | 0.727 |
| Luminal_Index | 0.0895 | 0.546 | 0.115 | 0.305 | 0.659 |
| ER_PGR_avg | 0.0167 | 0.61 | 0.174 | 0.396 | 0.675 |

Columns BH-BR

| | Ganetespib_ | Ganetespib_ | | | | |
|---|---|---|---|---|---|---|
| | HR+HER2−: | HR+HER2−: | TN.adj.Tx: | TN.adj.TX: | TN.adj.TX: | Ctr_TN: |
| | LR p | BH LR p | OR/1SD | LR p | BH LR p | OR/1SD |
| ICSS_score | 0.0144 | 0.0749 | 1.69 | 1.06E−05 | 0.000316 | 1.44 |
| Chemokine12_score | 0.0174 | 0.085 | 3.76 | 4.19E−08 | 0.000144 | 1.43 |
| Module5_TcellBcell_score | 0.0436 | 0.154 | 1.69 | 1.64E−05 | 0.000454 | 1.36 |
| STAT1_sig | 0.0106 | 0.0601 | 1.66 | 1.03E−05 | 0.000316 | 1.37 |
| Module3_IFN_score | 0.105 | 0.288 | 1.37 | 0.0052 | 0.0364 | 1.25 |
| Dendritic_cells | 0.249 | 0.48 | 1.61 | 3.97E−05 | 9.00E−04 | 0.978 |
| B_cells | 0.597 | 0.787 | 1.37 | 0.00729 | 0.0464 | 1.08 |
| Mast_cells | 0.0487 | 0.17 | 0.904 | 0.51 | 0.718 | 1.01 |
| Module11_Prolif_score | 0.328 | 0.565 | 1.12 | 0.387 | 0.616 | 1.17 |
| MP_ index_adj*(−1) | 0.529 | 0.732 | 1.55 | 0.0331 | 0.128 | 1.03 |
| Basal_Index | 0.861 | 0.951 | 1.22 | 0.462 | 0.675 | 0.305 |
| PARPi7_score | 0.364 | 0.591 | 1.13 | 0.248 | 0.479 | 1.06 |
| PARPi7_plus_MP2 | 0.327 | 0.565 | 1.18 | 0.15 | 0.365 | 1.07 |
| VCpred_TN | 0.0112 | 0.062 | 1.68 | 1.72E−07 | 9.29E−06 | 1.58 |
| STMN1_dat | 0.252 | 0.484 | 1.31 | 0.0392 | 0.144 | 0.933 |
| HER2_Index | 0.73 | 0.867 | 0.917 | 0.859 | 0.949 | 0.926 |
| Mod7_ERBB2 | 0.278 | 0.509 | 1.46 | 0.328 | 0.565 | 1.46 |
| ERBB2.Y1248 | NA | NA | 6.59 | 0.0948 | 0.266 | 171 |
| EGFR.Y1173 | NA | NA | 8.03 | 0.0387 | 0.143 | 125 |
| mTOR.S2448 | NA | NA | 0.907 | 0.519 | 0.723 | 1.54 |
| IGF1R_dat | 0.411 | 0.638 | 0.986 | 0.932 | 0.995 | 0.655 |
| TIE2.Y992 | NA | NA | 0.968 | 0.807 | 0.915 | 0.754 |
| Mod10_ECM | 0.632 | 0.808 | 0.918 | 0.447 | 0.663 | 1.42 |
| RPL24_dat | 0.842 | 0.936 | 0.874 | 0.193 | 0.41 | 1.05 |
| LYMPHS_PCA_16704732 | 0.445 | 0.661 | 0.841 | 0.179 | 0.396 | 1.06 |
| Luminal_Index | 0.315 | 0.554 | 0.512 | 0.0835 | 0.245 | 0.419 |
| ER_PGR_avg | 0.408 | 0.636 | 0.818 | 0.418 | 0.646 | 0.649 |

| | | Ctr_TN: BH | N_TN: | | N_TN: BH |
|---|---|---|---|---|---|
| | Ctr_TN: LR p | LR p | OR/1SD | N_TN: LR p | LR p |
| ICSS_score | 0.185 | 0.402 | 1.7 | 0.152 | 0.367 |
| Chemokine12_score | 0.23 | 0.457 | 2.09 | 0.0433 | 0.153 |
| Module5_TcellBcell_score | 0.292 | 0.526 | 1.91 | 0.0832 | 0.245 |
| STAT1_sig | 0.245 | 0.474 | 1.79 | 0.0786 | 0.236 |
| Module3_IFN_score | 0.414 | 0.642 | 1.34 | 0.357 | 0.588 |
| Dendritic_cells | 0.94 | 0.997 | 2.63 | 0.0245 | 0.105 |
| B_cells | 0.745 | 0.876 | 1.39 | 0.565 | 0.764 |
| Mast_cells | 0.973 | 1 | 0.728 | 0.605 | 0.79 |
| Module11_Prolif_score | 0.614 | 0.796 | 1.18 | 0.722 | 0.867 |
| MP_ index_adj*(−1) | 0.937 | 0.997 | 4.08 | 0.156 | 0.372 |
| Basal_Index | 0.036 | 0.137 | 2.57 | 0.326 | 0.565 |
| PARPi7_score | 0.807 | 0.915 | 1.26 | 0.552 | 0.754 |
| PARPi7_plus_MP2 | 0.805 | 0.915 | 1.37 | 0.458 | 0.672 |
| VCpred_TN | 0.0904 | 0.259 | 1.83 | 0.101 | 0.28 |
| STMN1_dat | 0.821 | 0.924 | 2.52 | 0.0428 | 0.152 |
| HER2_Index | 0.936 | 0.997 | 2.85 | 0.333 | 0.569 |
| Mod7_ERBB2 | 0.698 | 0.853 | 2.19 | 0.573 | 0.77 |
| ERBB2.Y1248 | 0.00532 | 0.0369 | >10 | 0.0421 | 0.15 |
| EGFR.Y1173 | 0.00192 | 0.017 | >10 | 0.0549 | 0.184 |
| mTOR.S2448 | 0.189 | 0.406 | 1.77 | 0.184 | 0.402 |
| IGF1R_dat | 0.258 | 0.488 | 2.48 | 0.186 | 0.402 |
| TIE2.Y992 | 0.527 | 0.731 | 0.066 | 0.0101 | 0.059 |
| Mod10_ECM | 0.179 | 0.396 | 0.629 | 0.181 | 0.398 |
| RPL24_dat | 0.814 | 0.919 | 1.54 | 0.223 | 0.448 |
| LYMPHS_PCA_16704732 | 0.83 | 0.926 | 2.45 | 0.106 | 0.289 |
| Luminal_Index | 0.358 | 0.588 | 1.65 | 0.726 | 0.867 |
| ER_PGR_avg | 0.453 | 0.668 | 0.668 | 0.627 | 0.805 |

Columns BS-CC

| | MK2206_TN: | MK2206_TN: | MK2206_TN: | AMG386_TN: | AMG386_TN: | AMG386_TN: |
|---|---|---|---|---|---|---|
| | OR/1SD | LR p | BH LR p | OR/1SD | LR p | BH LR p |
| ICSS_score | 1.46 | 0.397 | 0.624 | 1.97 | 0.0347 | 0.132 |
| Chemokine12_score | 1.89 | 0.187 | 0.403 | 2.48 | 0.00916 | 0.0555 |
| Module5_TcellBcell_score | 1.32 | 0.537 | 0.741 | 2.45 | 0.00687 | 0.0448 |
| STAT1_sig | 1.58 | 0.309 | 0.546 | 2.39 | 0.00869 | 0.0536 |
| Module3_IFN_score | 1.97 | 0.154 | 0.369 | 1.6 | 0.196 | 0.411 |
| Dendritic_cells | 1.13 | 0.755 | 0.882 | 2.77 | 0.00131 | 0.0135 |
| B_cells | 1.08 | 0.838 | 0.933 | 1.6 | 0.122 | 0.318 |
| Mast_cells | 1.11 | 0.79 | 0.904 | 0.843 | 0.661 | 0.826 |
| Module11_Prolif_score | 1.37 | 0.44 | 0.66 | 0.954 | 0.873 | 0.959 |
| MP_ index_adj*(−1) | 1.74 | 0.343 | 0.573 | 0.973 | 0.944 | 0.999 |
| Basal_Index | 1.88 | 0.501 | 0.709 | 1.41 | 0.512 | 0.718 |
| PARPi7_score | 0.56 | 0.158 | 0.374 | 1.47 | 0.172 | 0.393 |
| PARPi7_plus_MP2 | 0.631 | 0.265 | 0.496 | 1.46 | 0.203 | 0.421 |
| VCpred_TN | 1.05 | 0.904 | 0.976 | 2.38 | 0.0205 | 0.0926 |
| STMN1_dat | 1.06 | 0.866 | 0.953 | 1.23 | 0.512 | 0.718 |
| HER2_Index | 261 | 0.135 | 0.34 | 0.141 | 0.26 | 0.49 |
| Mod7_ERBB2 | 1.9 | 0.6 | 0.788 | 0.168 | 0.129 | 0.33 |
| ERBB2.Y1248 | <0.01 | 0.0772 | 0.235 | <0.01 | 0.0834 | 0.245 |
| EGFR.Y1173 | 0.149 | 0.746 | 0.877 | <0.01 | 0.0736 | 0.227 |
| mTOR.S2448 | 0.394 | 0.0379 | 0.141 | 0.761 | 0.337 | 0.57 |
| IGF1R_dat | 1.96 | 0.244 | 0.473 | 0.964 | 0.944 | 0.999 |
| TIE2.Y992 | 0.979 | 0.97 | 1 | 0.947 | 0.805 | 0.915 |
| Mod10_ECM | 1.04 | 0.924 | 0.992 | 1.15 | 0.57 | 0.767 |
| RPL24_dat | 0.749 | 0.596 | 0.787 | 0.947 | 0.852 | 0.944 |
| LYMPHS_PCA_16704732 | 1.5 | 0.406 | 0.633 | 1.05 | 0.893 | 0.968 |
| Luminal_Index | 2.91 | 0.387 | 0.616 | 0.511 | 0.489 | 0.699 |
| ER_PGR_avg | 0.813 | 0.817 | 0.921 | 0.806 | 0.717 | 0.866 |

| | VC_TN: | | VC_TN: BH | Pembro_TN: | Pembro_TN: |
|---|---|---|---|---|---|
| | OR/1SD | VC_TN: LR p | LR p | OR/1SD | LR p |
| ICSS_score | 2.29 | 0.0331 | 0.128 | 2.58 | 0.011 |
| Chemokine12_score | 1.91 | 0.0807 | 0.241 | 5.8 | 0.00113 |
| Module5_TcellBcell_score | 1.87 | 0.0906 | 0.259 | 4.62 | 0.00256 |
| STAT1_sig | 1.69 | 0.111 | 0.296 | 7.2 | 0.000623 |
| Module3_IFN_score | 1.31 | 0.433 | 0.655 | 1.93 | 0.0636 |
| Dendritic_cells | 2.58 | 0.0143 | 0.0747 | 4.36 | 0.000766 |
| B_cells | 1.81 | 0.0982 | 0.274 | 3.94 | 0.0528 |
| Mast_cells | 1.24 | 0.586 | 0.781 | 0.811 | 0.722 |
| Module11_Prolif_score | 1.13 | 0.829 | 0.926 | 1.15 | 0.728 |
| MP_ index_adj*(−1) | 2.5 | 0.239 | 0.466 | 2.54 | 0.257 |
| Basal_Index | 3.92 | 0.432 | 0.655 | 3.98 | 0.118 |
| PARPi7_score | 3.15 | 0.00507 | 0.0358 | 0.829 | 0.589 |
| PARPi7_plus_MP2 | 3.74 | 0.00378 | 0.0288 | 0.866 | 0.698 |
| VCpred_TN | 9.72 | 3.64E−06 | 0.000142 | 2.27 | 0.0319 |
| STMN1_dat | 1.56 | 0.275 | 0.506 | 1.44 | 0.426 |
| HER2_Index | 0.438 | 0.364 | 0.591 | 0.771 | 0.939 |
| Mod7_ERBB2 | 1.82 | 0.656 | 0.825 | 4 | 0.211 |
| ERBB2.Y1248 | 4.41 | 0.515 | 0.72 | 1.63 | 0.88 |
| EGFR.Y1173 | 0.902 | 0.959 | 1 | 1.45 | 0.9 |
| mTOR.S2448 | 0.559 | 0.149 | 0.364 | 1.43 | 0.551 |
| IGF1R_dat | 0.935 | 0.889 | 0.967 | 0.494 | 0.208 |
| TIE2.Y992 | 1.21 | 0.438 | 0.659 | NA | NA |
| Mod10_ECM | 1.07 | 0.877 | 0.96 | 0.376 | 0.0308 |
| RPL24_dat | 0.342 | 0.00709 | 0.0454 | 0.734 | 0.393 |
| LYMPHS_PCA_16704732 | 0.0954 | 0.000201 | 0.00304 | 0.898 | 0.75 |
| Luminal_Index | 0.39 | 0.374 | 0.604 | 0.0986 | 0.188 |
| ER_PGR_avg | 0.954 | 0.933 | 0.995 | 0.792 | 0.863 |

Columns CD-CM

| | Pembro_TN: | Ganitumab_TN: | Ganitumab_TN: | Ganitumab_TN: | Ganetespib_TN: |
|---|---|---|---|---|---|
| | BH LR p | OR/1SD | LR p | BH LR p | OR/1SD |
| ICSS_score | 0.0614 | 1.6 | 0.145 | 0.357 | 1.25 |
| Chemokine12_score | 0.012 | 1.39 | 0.261 | 0.491 | 1.16 |
| Module5_TcellBcell_score | 0.021 | 1.45 | 0.226 | 0.453 | 1.19 |
| STAT1_sig | 0.00768 | 3.42 | 0.196 | 0.411 | 1.15 |
| Module3_IFN_score | 0.206 | 1.33 | 0.355 | 0.586 | 1.1 |
| Dendritic_cells | 0.00886 | 1.44 | 0.228 | 0.454 | 0.868 |
| B_cells | 0.178 | 3.84 | 0.319 | 0.557 | 3.16 |
| Mast_cells | 0.867 | 0.613 | 0.331 | 0.569 | 0.627 |
| Module11_Prolif_score | 0.867 | 1.7 | 0.139 | 0.346 | 0.598 |
| MP_ index_adj*(−1) | 0.488 | 2.17 | 0.199 | 0.415 | 4.81 |
| Basal_Index | 0.31 | 0.5 | 0.447 | 0.663 | $.69 |
| PARPi7_score | 0.782 | 1.19 | 0.549 | 0.752 | 0.873 |
| PARPi7_plus_MP2 | 0.853 | 1.31 | 0.396 | 0.623 | 0.917 |
| VCpred_TN | 0.125 | 1.65 | 0.106 | 0.289 | 3.31 |
| STMN1_dat | 0.65 | 1.75 | 0.131 | 0.332 | 1.14 |
| HER2_Index | 0.997 | 0.111 | 0.366 | 0.593 | 46.8 |
| Mod7_ERBB2 | 0.43 | 0.873 | 0.886 | 0.966 | 3.57 |
| ERBB2.Y1248 | 0.961 | NA | NA | NA | NA |
| EGFR.Y1173 | 0.974 | NA | NA | NA | NA |
| mTOR.S2448 | 0.754 | NA | NA | NA | NA |
| IGF1R_dat | 0.425 | 0.494 | 0.11 | 0.296 | 2.74 |
| TIE2.Y992 | NA | NA | NA | NA | NÅ |
| Mod10_ECM | 0.123 | 0.525 | 0.0348 | 0.132 | 1.18 |
| RPL24_dat | 0.622 | 1.51 | 0.333 | 0.569 | 0.538 |
| LYMPHS_PCA_16704732 | 0.879 | 0.911 | 0.811 | 0.918 | 0.445 |
| Luminal_Index | 0.405 | 0.599 | 0.546 | 0.751 | 0.149 |
| ER_PGR_avg | 0.951 | 1.44 | 0.496 | 0.706 | 0.158 |
| | | | HR+HER2+. | HR+HER2+. | HR+HER2+. |
| | Ganetespib_ | Ganetespib_ | adj.Tx: | adj.Tx: | adj.Tx: |
| | TN: LR p | TN: BH LR p | OR/1SD | LR p | BH LR p |
| ICSS_score | 0.461 | 0.675 | 1.79 | 0.00327 | 0.0254 |
| Chemokine12_score | 0.635 | 0.808 | 1.98 | 0.00064 | 0.0078 |
| Module5_TcellBcell_score | 0.582 | 0.778 | 1.77 | 0.00249 | 0.0206 |
| STAT1_sig | 0.622 | 0.801 | 1.66 | 0.0147 | 0.0758 |
| Module3_IFN_score | 0.708 | 0.859 | 1.19 | 0.338 | 0.57 |
| Dendritic_cells | 0.633 | 0.808 | 1.45 | 0.0508 | 0.175 |
| B_cells | 0.637 | 0.81 | 1.96 | 0.000369 | 0.00487 |
| Mast_cells | 0.342 | 0.573 | 0.824 | 0.395 | 0.622 |
| Module11_Prolif_score | 0.236 | 0.462 | 2.2 | 0.000886 | 0.00975 |
| MP_ index_adj*(−1) | 0.0834 | 0.245 | 3.2 | 1.62E−06 | 7.35E−09 |
| Basal_Index | 0.16 | 0.374 | 2.63 | 0.0104 | 0.0599 |
| PARPi7_score | 0.658 | 0.825 | 1.4 | 0.148 | 0.362 |
| PARPi7_plus_MP2 | 0.792 | 0.904 | 1.99 | 0.00702 | 0.0452 |
| VCpred_TN | 0.404 | 0.631 | 1.68 | 0.00658 | 0.0431 |
| STMN1_dat | 0.734 | 0.869 | 1.95 | 0.00441 | 0.0325 |
| HER2_Index | 0.177 | 0.396 | 2.84 | 8.22E−08 | 4.66E−06 |
| Mod7_ERBB2 | 0.207 | 0.425 | 3.62 | 1.64E−09 | 1.37E−07 |
| ERBB2.Y1248 | NA | NA | 1.96 | 1.75E:05 | 0.000462 |
| EGFR.Y1173 | NA | NA | 1.7 | 5.22E−05 | 0.00112 |
| mTOR.S2448 | NA | NA | 1.92 | 0.004 | 0.03 |
| IGF1R_dat | 0.0272 | 0.113 | 0.435 | 0.000132 | 0.00237 |
| TIE2.Y992 | NA | NA | 1.34 | 0.28 | 0.51 |
| Mod10_ECM | 0.608 | 0.792 | 1.03 | 0.893 | 0.968 |
| RPL24_dat | 0.0296 | 0.119 | 1.01 | 0.977 | 1 |
| LYMPHS_PCA_16704732 | 0.0187 | 0.0887 | 0.606 | 0.023 | 0.101 |
| Luminal_Index | 0.104 | 0.286 | 0.242 | 5.32E−09 | 3.77E−07 |
| ER_PGR_avg | 0.104 | 0.286 | 0.302 | 8.32E−06 | 0.00027 |

Columns CN-CW

| | Ctr_HR+HER2+: | Ctr_HR+HER2+: | Ctr_HR+HER2+: | N_HR+HER2+: | N_HR+HER2+: |
|---|---|---|---|---|---|
| | OR/1SD | LR p | BH LR p | OR/1SD | LR p |
| ICSS_score | 1.48 | 0.56 | 0.763 | 2.22 | 0.039 |
| Chemokine12_score | 1.01 | 0.993 | 1 | 3.7 | 0.00295 |
| Module5_TcellBcell_score | 0.974 | 0.967 | 1 | 3.02 | 0.00533 |
| STAT1_sig | 0.816 | 0.758 | 0.883 | 2.92 | 0.0189 |
| Module3_IFN_score | 0.587 | 0.459 | 0.672 | 1.55 | 0.208 |
| Dendritic_cells | 0.513 | 0.387 | 0.616 | 1.67 | 0.179 |
| B_cells | 1.71 | 0.421 | 0.647 | 2.59 | 0.00592 |
| Mast_cells | 0.63 | 0.688 | 0.847 | 1.6 | 0.279 |
| Module11_Prolif_score | 2.12 | 0.353 | 0.584 | 1.77 | 0.233 |
| MP_ index_adj*(−1) | 3.2 | 0.152 | 0.367 | 2.66 | 0.0278 |
| Basal_Index | 7.8 | 0.236 | 0.462 | 2.77 | 0.0709 |
| PARPi7_score | 1.96 | 0.422 | 0.648 | 1.36 | 0.515 |
| PARPi7_plus_MP2 | 2.67 | 0.258 | 0.488 | 1.95 | 0.195 |
| VCpred_TN | 1.01 | 0.992 | 1 | 2.29 | 0.0251 |
| STMN1_dat | 1.55 | 0.583 | 0.778 | 1.18 | 0.749 |
| HER2_Index | 2.51 | 0.137 | 0.343 | 2.6 | 0.0327 |
| Mod7_ERBB2 | 2.72 | 0.154 | 0.369 | 3.87 | 0.00177 |
| ERBB2.Y1248 | 2.47 | 0.452 | 0.668 | 1.76 | 0.00862 |
| EGFR.Y1173 | 1.65 | 0.683 | 0.843 | 1.63 | 0.0139 |
| mTOR.S2448 | 0.47 | 0.402 | 0.629 | 1.78 | 0.194 |
| IGF1R_dat | 0.00936 | 0.0177 | 0.0861 | 0.376 | 0.0568 |
| TIE2.Y992 | 0.782 | 0.711 | 0.86 | 1.65 | 0.194 |
| Mod10_ECM | 0.443 | 0.487 | 0.699 | 1.14 | 0.72 |
| RPL24_dat | 1.24 | 0.752 | 0.879 | 0.584 | 0.244 |
| LYMPHS_PCA_16704732 | 1.32 | 0.759 | 0.883 | 0.25 | 0.00322 |
| Luminal_Index | 0.121 | 0.0511 | 0.175 | 0.178 | 0.00106 |
| ER_PGR_avg | 0.126 | 0.0655 | 0.209 | 0.232 | 0.0106 |
| | N_HR+ | MK2206_HR+ | | MK2206_HR+ | AMG386_ |
| | HER2+: | HER2+: | MK2206_HR+ | HER2+: | HR+HER2+: |
| | BH LR p | OR/1SD | HER2+: LR p | BH LR p | OR/1SD |
| ICSS_score | 0.144 | 1.57 | 0.424 | 0.65 | 1.33 |
| Chemokine12_score | 0.0237 | 3.35 | 0.631 | 0.808 | 1.49 |
| Module5_TcellBcell_score | 0.0369 | 1.47 | 0.483 | 0.697 | 1.35 |
| STAT1_sig | 0.0893 | 0.985 | 0.979 | 1 | 2.13 |
| Module3_IFN_score | 0.425 | 0.556 | 0.385 | 0.616 | 1.37 |
| Dendritic_cells | 0.396 | 1.63 | 0.418 | 0.646 | 1.26 |
| B_cells | 0.0397 | 4.19 | 0.0402 | 0.146 | 0.723 |
| Mast_cells | 0.509 | 0.42.8 | 0.349 | 0.579 | 1.45 |
| Module11_Prolif_score | 0.46 | 1.59 | 0.432 | 0.655 | 1.76 |
| MP_ index_adj*(−1) | 0.114 | 2.34 | 0.232 | 0.46 | 0.565 |
| Basal_Index | 0.223 | 50 | 0.0938 | 0.265 | 0.421 |
| PARPi7_score | 0.72 | 0.672 | 0.564 | 0.764 | 7.28 |
| PARPi7_plus_MP2 | 0.411 | 0.841 | 0.813 | 0.919 | 6.07 |
| VCpred_TN | 0.107 | 1.23 | 0.698 | 0.853 | 2.53 |
| STMN1_dat | 0.878 | 3.17 | 0.0294 | 0.119 | 0.584 |
| HER2_Index | 0.128 | 0.786 | 0.749 | 0.878 | 1.38 |
| Mod7_ERBB2 | 0.0165 | 3.21 | 0.273 | 0.505 | 1.92 |
| ERBB2.Y1248 | 0.0534 | 1.65 | 0.259 | 0.489 | 1.35 |
| EGFR.Y1173 | 0.073 | 1.38 | 0.287 | 0.518 | 0.912 |
| mTOR.S2448 | 0.41 | 6.21 | 0.0179 | 0.0864 | 4.21 |
| IGF1R_dat | 0.188 | 0.347 | 0.141 | 0.349 | 0.918 |
| TIE2.Y992 | 0.41 | 0.597 | 0.634 | 0.808 | 29.2 |
| Mod10_ECM | 0.867 | 0.569 | 0.322 | 0.561 | 2.88 |
| RPL24_dat | 0.473 | 1.52 | 0.463 | 0.676 | 0.634 |
| LYMPHS_PCA_16704732 | 0.0254 | 1.18 | 0.789 | 0.904 | 0.788 |
| Luminal_Index | 0.0113 | 0.58 | 0.561 | 0.763 | 1.06 |
| ER_PGR_avg | 0.0601 | 1.17 | 0.83 | 0.926 | 0.874 |

Columns CX-DG

| | AMG386_ | AMG386_ | Pertuzumab_ | Pertuzumab_ | Pertuzumab_ |
|---|---|---|---|---|---|
| | HR+HER24: | HR+HER2+: | HR+HER2+: | HR+HER2+: | HR+HER2+: |
| | LR p | BH LR p | OR/1SD | LR p | BH LR p |
| ICSS_score | 0.689 | 0.847 | 1.99 | 0.0783 | 0.236 |
| Chemokine12_score | 0.648 | 0.819 | 1.83 | 0.0948 | 0.266 |
| Module5_TcellBcell_score | 0.679 | 0.841 | 1.65 | 0.146 | 0.358 |
| STAT1_sig | 0.385 | 0.616 | 1.61 | 0.173 | 0.395 |
| Module3_IFN_score | 0.608 | 0.792 | 1.13 | 0.72 | 0.867 |
| Dendritic_cells | 0.726 | 0.867 | 1.59 | 0.279 | 0.509 |
| B_cells | 0.628 | 0.806 | 1.83 | 0.129 | 0.33 |
| Mast_cells | 0.654 | 0.823 | 0.747 | 0.568 | 0.766 |
| Module11_Prolif_score | 0.588 | 0.782 | 1.79 | 0.268 | 0.501 |
| MP_ index_adj*(−1) | 0.579 | 0.773 | 4.07 | 0.0153 | 0.0778 |
| Basal_Index | 0.583 | 0.778 | 2.96 | 0.22 | 0.445 |
| PARPi7_score | 0.029 | 0.118 | 0.715 | 0.543 | 0.747 |
| PARPi7_plus_MP2 | 0.05 | 0.172 | 1.05 | 0.928 | 0.994 |
| VCpred_TN | 0.177 | 0.396 | 1.61 | 0.228 | 0.454 |
| STMN1_dat | 0.643 | 0.814 | 1.62 | 0.299 | 0.533 |
| HER2_Index | 0.594 | 0.786 | 3.39 | 0.00183 | 0.0167 |
| Mod7_ERBB2 | 0.257 | 0.488 | 3.9 | 0.00153 | 0.0147 |
| ERBB2.Y1248 | 0.589 | 0.782 | 2.29 | 0.0873 | 0.253 |
| EGFR.Y1173 | 0.869 | 0.956 | 1.65 | 0.185 | 0.402 |
| mTOR.S2448 | 0.0812 | 0.242 | 1.42 | 0.596 | 0.787 |
| IGF1R_dat | 0.855 | 0.947 | 0.447 | 0.0574 | 0.189 |
| TIE2.Y992 | 0.0408 | 0.146 | NA | NA | NA |
| Mod10_ECM | 0.156 | 0.372 | 1.74 | 0.224 | 1.45 |
| RPL24_dat | 0.5 | 0.709 | 0.981 | 0.971 | 1 |
| LYMPHS_PCA_16704732 | 0.703 | 0.856 | 0.84 | 0.728 | 0.867 |
| Luminal_Index | 0.939 | 0.997 | 0.219 | 0.000718 | 0.00839 |
| ER_PGR_avg | 0.83 | 0.926 | 0.146 | 0.00214 | 0.0186 |
| | TDM1/P_HR+ | | TDM1/P_HR+ | HR−HER2+. | HR−HER2+. |
| | HER2+: | TDMI/P_HR+ | HER2+: | adj.Tx: | adj.Tx: |
| | OR/1SD | HER2+: LR p | BH LR p | OR/1SD | LR p |
| ICSS_score | 1.54 | 0.339 | 0.57 | 1.47 | 0.109 |
| Chemokine12_score | 2.31 | 0.0936 | 0.265 | 1.28 | 0.382 |
| Module5_TcellBcell_score | 1.72 | 0.236 | 0.462 | 1.19 | 0.495 |
| STAT1_sig | 1.94 | 0.258 | 0.488 | 1.11 | 0.723 |
| Module3_IFN_score | 1.47 | 0.365 | 0.592 | 0.765 | 0.251 |
| Dendritic_cells | 1.54 | 0.223 | 0.448 | 1.53 | 0.127 |
| B_cells | 1.85 | 0.158 | 0.374 | 1.45 | 0.0869 |
| Mast_cells | 0.443 | 0.0713 | 0.223 | 0.908 | 0.733 |
| Module11_Prolif_score | 4.1 | 0.00311 | 0.0248 | 1.14 | 0.649 |
| MP_ index_adj*(−1) | 6.63 | 0.00029 | 0.00396 | 0.904 | 0.77 |
| Basal_Index | 1.93 | 0.401 | 0.628 | 0.415 | 0.0166 |
| PARPi7_score | 1.83 | 0.192 | 0.409 | 0.755 | 0.339 |
| PARPi7_plus_MP2 | 3.12 | 0.0258 | 0.108 | 0.704 | 0.297 |
| VCpred_TN | 1.5 | 0.338 | 0.57 | 1.29 | 0.283 |
| STMN1_dat | 4.25 | 0.0123 | 0.0671 | 0.924 | 0.756 |
| HER2_Index | 4.28 | 4.64E−05 | 0.00101 | 1.32 | 0.236 |
| Mod7_ERBB2 | 4.92 | 0.000244 | 0.00342 | 1.45 | 0.138 |
| ERBB2.Y1248 | 4.23 | 0.00257 | 0.021 | 1.53 | 0.0273 |
| EGFR.Y1173 | 13.8 | 1.00E−04 | 0.00195 | 1.69 | 0.0153 |
| mTOR.S2448 | 1.89 | 0.11 | 0.296 | 1.08 | 0.786 |
| IGF1R_dat | 0.331 | 0.0194 | 0.0902 | 0.816 | 0.641 |
| TIE2.Y992 | NA | NA | NA | 0.916 | 0.78 |
| Mod10_ECM | 0.652 | 0.282 | 0.513 | 0.727 | 0.22 |
| RPL24_dat | 1.52 | 0.359 | 0.589 | 1.72 | 0.0501 |
| LYMPHS_PCA_16704732 | 0.535 | 0.181 | 0.398 | 1.88 | 0.0514 |
| Luminal_Index | 0.114 | 0.000154 | 0.00251 | 2.46 | 0.274 |
| ER_PGR_avg | 0.0851 | 0.00146 | 0.0142 | 1.78 | 0.265 |

Columns DH-DR

| | HR−HER2+. | Ctr_HR− | Ctr_HR− | Ctr_HR− | N_HR− | |
|---|---|---|---|---|---|---|
| | adj.Tx: | HER2+: | HER2+: | HER2+: | HER2+: | N_HR−HER2+: |
| | BH LR p | OR/1SD | LR p | BH LR p | OR/1SD | LR p |
| ICSS_score | 0.296 | 12.2 | 0.00629 | 0.0419 | 0.8 | 0.561 |
| Chemokine12_score | 0.614 | 20 | 0.00555 | 0.0377 | 0.589 | 0.253 |
| Module5_TcellBcell_score | 0.705 | 9.06 | 0.0164 | 0.0819 | 0.681 | 0.338 |
| STAT1_sig | 0.867 | 27.5 | 0.0163 | 0.0819 | 0.585 | 0.254 |
| Module3_IFN_score | 0.483 | >10 | 0.0035 | 0.0268 | 0.44 | 0.0493 |
| Dendritic_cells | 0.328 | 18.8 | 0.0128 | 0.0691 | 1.03 | 0.953 |
| B_cells | 0.252 | 3.06 | 0.072 | 0.224 | 1.1 | 0.779 |
| Mast_cells | 0.869 | 2.02 | 0.311 | 0.548 | 0.768 | 0.669 |
| Module11_Prolif_score | 0.82 | 1.03 | 0.978 | 1 | 1.5 | 0.426 |
| MP_ index_adj*(−1) | 0.891 | 0.318 | 0.207 | 0.425 | 1.2 | 0.744 |
| Basal_Index | 0.0822 | 0.432 | 0.304 | 0.539 | 0.707 | 0.602 |
| PARPi7_score | 0.57 | 0.604 | 0.356 | 0.587 | 0.658 | 0.565 |
| PARPi7_plus_MP2 | 0.532 | 0.524 | 0.277 | 0.508 | 0.64 | 0.617 |
| VCpred_TN | 0.514 | >10 | 5.40E−05 | 0.00113 | 0.759 | 0.42 |
| STMN1_dat | 0.882 | 0.503 | 0.433 | 0.655 | 1.12 | 0.823 |
| HER2_Index | 0.462 | 0.827 | 0.713 | 0.862 | 1.44 | 0.511 |
| Mod7_ERBB2 | 0.345 | 0.67 | 0.453 | 0.668 | 1.47 | 0.456 |
| ERBB2.Y1248 | 0.113 | 0.988 | 0.977 | 1 | 1.54 | 0.36 |
| EGFR.Y1173 | 0.0778 | 0.993 | 0.988 | 1 | 1.28 | 0.629 |
| mTOR.S2448 | 0.902 | 1.27 | 0.726 | 0.867 | 0.752 | 0.47 |
| IGF1R_dat | 0.813 | 0.708 | 0.617 | 0.798 | 1.46 | 0.643 |
| TIE2.Y992 | 0.898 | 1.91 | 0.43 | 0.655 | 0.586 | 0.24 |
| Mod10_ECM | 0.445 | 0.604 | 0.488 | 0.699 | 0.668 | 0.389 |
| RPL24_dat | 0.196 | 2.44 | 0.285 | 0.517 | 1.84 | 0.208 |
| LYMPHS_PCA_16704732 | 0.176 | 5.08 | 0.19 | 0.407 | 1.45 | 0.57 |
| Luminal_Index | 0.505 | 0.113 | 0.304 | 0.539 | 1.59 | 0.737 |
| ER_PGR_avg | 0.496 | 0.985 | 0.984 | 1 | 4.48 | 0.157 |

| | N_HR− | MK2206_ | | MK2206_ | Pertuzumab_ |
|---|---|---|---|---|---|
| | HER2+: | HR−HER2+: | MK2206_HR− | HR−HER2+: | HR−HER2+: |
| | BH LR p | OR/1SD | HER2+: LR p | BH LR p | OR/1SD |
| ICSS_score | 0.763 | 2.34 | 0.151 | 0.367 | 1.45 |
| Chemokine12_score | 0.485 | 1.66 | 0.495 | 0.705 | 0.869 |
| Module5_TcellBcell_score | 0.57 | 1.68 | 0.456 | 0.67 | 1 |
| STAT1_sig | 0.486 | 1.14 | 0.848 | 0.941 | 1.23 |
| Module3_IFN_score | 0.171 | 0.535 | 0.294 | 0.528 | 0.748 |
| Dendritic_cells | 1 | 1.55 | 0.362 | 0.591 | 0.931 |
| B_cells | 0.898 | 1.99 | 0.186 | 0.402 | 1.46 |
| Mast_cells | 0.832 | 0.638 | 0.395 | 0.622 | 0.142 |
| Module11_Prolif_score | 0.65 | 0.852 | 0.829 | 0.926 | >10 |
| MP_ index_adj*(−1) | 0.876 | 0.738 | 0.681 | 0.841 | 89.4 |
| Basal_Index | 0.789 | 0.309 | 0.271 | 0.504 | 1.77 |
| PARPi7_score | 0.764 | 1.15 | 0.774 | 0.895 | 0.261 |
| PARPi7_plus_MP2 | 0.798 | 1.13 | 0.829 | 0.926 | 0.797 |
| VCpred_TN | 0.646 | 3.13 | 0.0802 | 0.24 | 2.09 |
| STMN1_dat | 0.925 | 1.37 | 0.56 | 0.763 | 122 |
| HER2_Index | 0.718 | 0.771 | 0.68 | 0.841 | 0.462 |
| Mod7_ERBB2 | 0.67 | 1.15 | 0.832 | 0.927 | 0.858 |
| ERBB2.Y1248 | 0.589 | 1.44 | 0.341 | 0.572 | 9.78 |
| EGFR.Y1173 | 0.806 | 2.52 | 0.065 | 0.208 | 8.97 |
| mTOR.S2448 | 0.683 | 3.05 | 0.0864 | 0.252 | 5.16 |
| IGF1R_dat | 0.814 | 1.19 | 0.891 | 0.968 | 0.386 |
| TIE2.Y992 | 0.468 | 0.791 | 0.706 | 0.858 | NA |
| Mod10_ECM | 0.618 | 0.776 | 0.669 | 0.832 | 0.167 |
| RPL24_dat | 0.425 | 2.86 | 0.17 | 0.389 | 2.48 |
| LYMPHS_PCA_16704732 | 0.767 | 2.87 | 0.116 | 0.306 | 8.59 |
| Luminal_Index | 0.871 | 38 | 0.0589 | 0.193 | 62.9 |
| ER_PGR_avg | 0.374 | 4.55 | 0.307 | 0.543 | 0.00793 |

Columns DS-DW

| | Pertuzumab_ | Pertuzumab_ | TDM1/P_HR− | | TDM1/P_HR− |
|---|---|---|---|---|---|
| | HR−HER2+: LR | HR−HER2+: | HER2+: | TDM1/P_HR− | HER2+: BH LR |
| | p | BH LR p | OR/1SD | HER2+: LR p | p |
| ICSS_score | 0.707 | 0.858 | 1.66 | 0.299 | 0.533 |
| Chemokine12_score | 0.882 | 0.983 | 2.21 | 0.227 | 0.454 |
| Module5_TcellBcell_score | 0.997 | 1 | 1.54 | 0.441 | 0.66 |
| STAT1_sig | 0.827 | 0.926 | 1.67 | 0.442 | 0.66 |
| Module3_IFN_score | 0.614 | 0.796 | 0.944 | 0.908 | 0.979 |
| Dendritic_cells | 0.932 | 0.995 | 1.45 | 0.604 | 0.79 |
| B_cells | 0.737 | 0.871 | 1.24 | 0.6 | 0.788 |
| Mast_cells | 0.0748 | 0.23 | 1.33 | 0.757 | 0.882 |
| Module11_Prolif_score | 0.00145 | 0.0142 | 0.173 | 0.168 | 0.387 |
| MP_ index_adj*(−1) | 0.00508 | 0.0358 | 0.0785 | 0.0852 | 0.249 |
| Basal_Index | 0.605 | 0.79 | 0.115 | 0.0231 | 0.102 |
| PARPi7_score | 0.317 | 0.556 | 0.615 | 0.7 | 0.854 |
| PARPi7_plus_MP2 | 0.875 | 0.96 | 0.358 | 0.441 | 0.66 |
| VCpred_TN | 0.489 | 0.699 | 0.925 | 0.895 | 0.969 |
| STMN1_dat | 0.00943 | 0.0566 | 0.445 | 0.142 | 0.621 |
| HER2_Index | 0.39 | 0.619 | 3.25 | 0.0199 | 0.0914 |
| Mod7_ERBB2 | 0.817 | 0.921 | 5.46 | 0.00756 | 0.0476 |
| ERBB2.Y1248 | 0.0727 | 0.225 | 111 | 0.00753 | 0.0476 |
| EGFR.Y1173 | 0.11 | 0.296 | 944 | 0.00554 | 0.0377 |
| mTOR.S2448 | 0.143 | 0.353 | 0.105 | 0.0552 | 0.184 |
| IGF1R_dat | 0.486 | 0.699 | 0.412 | 0.581 | 0.778 |
| TIE2.Y992 | NA | NA | NA | NA | NA |
| Mod10_ECM | 0.0302 | 0.121 | 1.38 | 0.61 | 0.793 |
| RPL24_dat | 0.36 | 0.589 | 2.4 | 0.304 | 0.539 |
| LYMPHS_PCA_16704732 | 0.105 | 0.288 | 3.18 | 0.287 | 0.518 |
| Luminal_Index | 0.318 | 0.556 | 0.374 | 0.732 | 0.868 |
| ER_PGR_avg | 0.135 | 0.34 | 4.64 | 0.344 | 0.573 |

TABLE 3
Overview of MAMMAPRINT® probes and signature genes.

| | Probe sequence | Gene | Ensemble ID | REF SEQ ID | Corr |
|---|---|---|---|---|---|
| 1 | CTGAGTGGTCAGAGATCTGTAAAGCATGACT | ALDH4A1 | ENSG00000159423 | NM_170726 | + |
| | TTCAAGGATGGTTCTTAGGGGACTGTGTA | | | | |
| 2 | AGGACTTGAATGAGGAAACCAACACTTTGAG | FGF18 | ENSG00000156427 | NM_003862 | + |
| | AAACCAAAGTCCTTTTTCCCAAAGGTTCT | | | | |
| 3 | GCCATTAAGATTTGGATGGGAAGTTATGGGT | CAPZB | ENSG00000077549 | NM_017765 | + |
| | AATGAGAATATAATGACATCTTGCAACAT | | | | |
| 4 | GATGGCCCAGCCTGTAAGATACTGTATATGC | BBC3 | ENSG00000105327 | NM_014417 | + |
| | GCTGCTGTAGATACCGGAATGAATTTTCT | | | | |
| 5 | GGCCTCACATTCTGCTCTGCTAAGTTTGGAG | EBF4 | ENSG00000088881 | XM_938882 | + |
| | AAAACAGAACAATAAACCAGATGCAGGTG | | | | |
| 6 | AAGTACTGGAATGTAATGGTTGAAATTCCTA | NA | NA | NT_022517 | + |
| | TTCAGTGATCTGGAAGAACTCTAATGTTC | | | | |
| 7 | CCAACGCACACCAGTCTTCTCAATCTGACTG | MYLIP | ENSG00000007944 | NM_013262 | + |
| | TAATCTAATCTGTTGTGCTTTTGTTGGAC | | | | |
| 8 | GGTTTAAAGCTGAAGAGGTTGAAGCTAAAAG | WISP1 | ENSG00000104415 | NM_003882 | + |
| | GAAAAGGTTGTTGTTAATGAATATCAGGC | | | | |
| 9 | GGCTAAAAGGGAAAAAGGATATGTGGAGAAT | GSTM3 | ENSG00000134202 | NM_000849 | + |
| | CATCAAGATATGAATTGAATCGCTGCGAT | | | | |
| 10 | CCTTTCAAACATGATCAAAGATTTCCCAATGT | RAB27B, | ENSG00000041353, | NM_004163 | + |
| | GATCTCATCATCATGGATACTCAATTTG | AC098848.1 | ENSG00000267112 | | |
| 11 | GGGGAACAATGAGGGCATTTCATGAACCATC | RTN4RL1 | ENSG00000185924 | NM_178568 | + |
| | TCAGGCACTTCTGCATCACGGAAGACCTG | | | | |
| 12 | TGCCTTGAGAATTTCAAAAGAGGTAATCAGG | ECI2 | ENSG00000198721 | NM_006117 | + |
| | AAAAGAGAGAGAGAAAAACTACACGCTGT | | | | |
| 13 | GTCTGGGATTAAGGGCAAATCTATTACTTTT | TGFB3 | ENSG00000119699 | NM_003239 | + |
| | GCAAACTGTCCTCTACATCAATTAACATC | | | | |
| 14 | TAAAAAAGAAATAGTCAGTGTTTTCCTCCTTT | STK32B | ENSG00000152953 | NM_018401 | + |
| | CAACCGAGACTATTTCTGGATTGTGTGC | | | | |
| 15 | TTTTCAGAAAGAAGTCTGGACCAGGCTGAAG | ECI2 | ENSG00000198721 | NM_206836 | + |
| | GCATTTGCAAAGCTTCCCCCAAATGTCTT | | | | |
| 16 | CCTCATTGCCTTATTCGGAGTACTATTATCCA | MS4A7, | ENSG00000166927; | NM_206939 | + |
| | ATATATGAAATCAAAGATTGTCTCCTGA | MS4A14 | ENSG00000110079 | | |
| 17 | TGGCATCATACAAAGAGCAGGAGAAGCAAAC | AP2B1 | ENSG00000006125 | NM_1030006 | + |
| | ACCCAGAACTCTTTTGCTGGTCAGAGATT | | | | |
| 18 | TCCAGACCTACCTTGTACGCACATAGACATTT | DHX58 | ENSG00000108771 | NM_024119 | + |
| | TCATATGCACTGGATGGAGTTAGGGAAA | | | | |
| 19 | ATCTTTGTTAATTATTTTGGGGAGTAGTTGGG | RAI2 | ENSG00000131831 | NM_021785 | + |
| | AAATGGAAAGGTGAATTGGCTCTAGAGG | | | | |
| 20 | GTTCATTTCCAGCCCTTTCTAGATCTGATCTT | HIPK2 | ENSG00000064393 | NT_007933 | + |
| | TTAGGGGGAAAGACAGCTTAAAATGTTC | | | | |
| 21 | TGAATGTCATGTTTATGTCATAGACGTAGAA | QDPR | ENSG00000151552 | NM_000320 | + |
| | AACGCATCCTTGAATTAAACTGCCTTAAC | | | | |
| 22 | TACTGGAGTAACTGAGTCGGGACGCTGAATC | ZG16B | ENSG00000162078 | NM_145252 | + |
| | TGAATCCACCAATAAATAAAGCTTCTGCA | | | | |
| 23 | CAGATTCCCCAGAAACTACCTTTTGCCCAAA | NEO1 | ENSG00000067141 | NM_002499 | + |
| | GAACATGCTCAGTATTTGGGGCATTTCCT | | | | |
| 24 | AGGCAGGGGTGGTGATTCATGCTGTGTGACT | ACADS | ENSG00000122971 | NM_000017 | + |
| | GACTGTGGGTAATAAACACACCTGTCCCC | | | | |
| 25 | TGGATTTCTAAACTGCTCAATTTTGACTCAAA | BTG2 | ENSG00000159388 | NM_006763 | + |
| | GGTGCTATTTACCAAACACTCTCCCTAC | | | | |
| 26 | CCAATCCAACAACTATAGGCTGGGTTAAATA | BBOF1, | ENSG00000119636, | NM_005589 | + |
| | AAAGGTCATTATTGTCTATATTCCAAGTG | ALDH6A1 | ENSG00000119711 | | |
| 27 | TCTACCACATTAAATTCTCCATTACATCTCAC | LYPD6, | ENSG0000018712 | NM_194317 | + |
| | TATTGGTAATGGCTTAAGTGTAAAGAGC | LINC00474 | ENSG00000204148 | | |
| 28 | TGAGGAATTCTTGTACGCAGTTTTCTTTGGCT | CIRBP | ENSG00000099622 | NM_001280 | + |
| | TTACGAGCCGATTAAAAGACCGTGTGAA | | | | |
| 29 | CTGGTCTTTGAAAGAAATGTACTACTAAAGA | AC07914, MATN3 | ENSG00000227210E | NM_002381 | + |
| | GCACTAGTTGTGAATTTAGGGTGTTAAAC | | NSG00000132031 | | |
| 30 | ATGATGGGAGAGCTCTGGCAGATGTCCCAAT | INPP5J | ENSG00000185133 | NM_014422 | + |
| | CCTGGAGGTCATCCATTAGGAATTAAATT | | | | |
| 31 | CAACTTGCTCTTTCATATGAGTTGGTCATAGC | FGD6 | ENSG00000180263 | NT_019546 | + |
| | ATGTAAGAACCAATCTTGAAATATCGTT | | | | |
| 32 | CCTGGATCAGAGTAAGAATGTCTTAAGAAGA | CACNAID, CHDH | ENSG00000157388, | NT_022517 | |
| | GGTTTGTAAGGTCTTCATAACAAAGTGGT | | ENSG00000016391 | | |
| 33 | CTCCTGGACTGCTTCTTTTGGCTCTCCGACAA | SDSL | ENSG00000139410 | NM_138432 | |
| | CTCCGGCCAATAAACACTTTCTGAATTG | | | | |
| 34 | AACCAACCCATAATTGCATTTTACTTGTCGTG | MINOS1, NBL1, | ENSG00000173436, | NM_001032363 | |
| | GTTCGATCTGATTGTATTGTCGAAGGAC | MINOS1-NBL1 | ENSG00000158747, | | |
| | | | ENSG00000270136 | | |
| 35 | ATTCCTTTATGAGCTCTCCATATCCTTCTTGA | PEX12 | ENSG00000108733 | NM_000286 | + |
| | GAAACTGGTTAAAAAAGGAATAGGGGTA | | | | |
| 36 | AGTGGGGGTTGTGTAAAGGGGAAGTCATCTT | ERGIC1 | ENSG00000113719 | NM_001031711 | + |
| | TTGAGATCCAGATAGACATGGTTTGTGCA | | | | |
| 37 | TCAGCTTAAGTACTTATTGTGGTAGTGAGTC | FBXO16 | ENSG00000214050 | NM_172366 | + |
| | CTACGGTATTTCAGTAAAAAGGAATTCAT | | | | |
| 38 | GGCAAGAGTTATCATAGAACAACAAAATAGA | ZNF385B | ENSG00000144331 | NM_152520 | + |
| | GTGGACTCTTTTAGAGCATCTATATCTGC | | | | |
| 39 | GGAGTTTCTGTTTAGGGCATTAAAAATTCCC | IP6K2 | ENSG00000068745 | NM_1005913 | + |
| | GCAAACTATAAAGAGCAATGTTTTCAGTC | | | | |
| 40 | ATAATTCTCTGTACAGGGGGGTTTGTGCTAT | MARCH8 | ENSG00000165406 | NM_145021 | + |
| | ACACTGGGATGTCTAATTGCAGCAATAAA | | | | |
| 41 | AGGACTTTAATCTTGGTGATGCCTTGGACAG | CMTM8, KRT18P15, | ENSG00000170293, | NM_199187 | + |
| | CAGCAACTCCATGCAAACCATCCAAAAGA | KRT18P34, KRT1 | ENSG00000234737, | | |
| | | 8P13, PCDH11Y, | ENSG00000244515, | | |
| | | KRT18P10 | ENSG00000214417, | | |
| | | | ENSG00000099715, | | |
| | | | ENSG00000214207 | | |
| 42 | GGGCAAAATGTATCACTCCAAACACTACTGA | RUNDC1 | ENSG00000198863 | NM_173079 | + |
| | TTCAGCATTGTTTTCATGTCTTAAAATTG | | | | |
| 43 | CTGGATGTTTAGCTTCTTACTGCAAAAACATA | TBC1D9 | ENSG00000109436 | NM_015130 | + |
| | AGTAAAACAGTCAACTTTACCATTTCCG | | | | |
| 44 | GGTAACTTGCAGGAATATTCTATTGGAAAAG | LETMD1 | ENSG00000050426 | NM_015416 | + |
| | ATAACAGGAAGTACAAGTGCTTCTTGACC | | | | |
| 45 | TCAATGGTTAGCAGAAGGGAGAAAAGAAAGC | RILPL2 | ENSG00000150977 | NP_659495 | + |
| | AGGAAAATGTGCTATTGAGATTCCAGTGG | | | | |
| 46 | CCTGGGTTTACAACGCTGTTAGGAAAATTAA | SEC14L2, | ENSG00000100003, | NM_012429 | + |
| | CCAATGAATAAAGCAACGTTCAGTGCGCA | AC004832.3 | ENSG00000249590 | | |
| 47 | TTTTTGTACCTTGTCACTATAACTACTTCCTA | KIAA1217 | ENSG00000120549 | NM_019590 | + |
| | GTCAAAGAACGAAATGTAACTGTTACCG | | | | |
| 48 | TTCTAGCTGTTATTTTGCTATTTGGCATTTAC | CCDC74A, | ENSG00000163040, | NM_207310 | + |
| | ATAAAAGCACACGATGAAGCAGGTATCG | MED15P9, | ENSG00000223760, | | |
| | | CCDC74B | ENSG00000152076 | | |
| 49 | TTGGGTTTATTTCCAGGTCACAGAATTGCTGT | TBX3 | ENSG00000135111 | NM_005996 | + |
| | TAACACTAGAAAACACACTTCCTGCACC | | | | |
| 50 | GAACAGCTCCTTACTCTGAGGAAGTTGATTC | FUT8 | ENSG00000033170 | NM_178157 | + |
| | TTATTTGATGGTGGTATTGTGACCACTGA | | | | |
| 51 | CTTTCTTATTTACTAAGAATTTGCCTGTTTGA | KIF3B | ENSG00000101350 | NM_004798 | + |
| | ATAAGAACAAAACGCTAAGGTGGGTAGC | | | | |
| 52 | CTAGAGAGCAGAAATAAAAAGCATGACTATT | PCAT7, FBP1 | ENSG00000231806, | NM_000507 | + |
| | TCCACCATCAAATGCTGTAGAATGCTTGG | | ENSG00000165140 | | |
| 53 | GTTCAGGGGCATCACCTACTTTGCTTACTTG | LBHD1, CSKMT | ENSG00000162194, | NM_024099 | + |
| | ATTCAAGGCTCTCATTAAAGACATTTTAG | | ENSG00000214756 | | |
| 54 | GTTGGTAGAGGGAGTATGATAAAATGTTTAA | KIAA1324 | ENSG00000116299 | NM_020775 | + |
| | ATCTCATTTGGTTACCTTGAGTCCTGGAA | | | | |
| 55 | AATTCAACAGTGTGGAAGCTTTAGGGGAACA | TMEM25 | ENSG00000149582 | NM_032780 | + |
| | TGGAGAAAGAAGGAGACCACATACCCCAA | | | | |
| 56 | CAAGTTGTGCAAAGTGAGAAAGATCTTTGTG | PIN4, RPS4X | ENSG00000102309, | NM_001007 | + |
| | GGCACAAAAGGAATCCCTCATCTGGTGAC | | ENSG00000198034 | | |
| 57 | CAAGAGAACCTGGAGAAAACTACCGTATTCA | STON2 | ENSG00000140022 | NT_026437 | + |
| | AGAGATTAATCAAAATCAGTGTTTTAGCC | | | | |
| 58 | CCGAATGACCTTAAAGGTGATCGGCTTTAAC | TENM3 | ENSG00000218336 | XM_940722 | + |
| | GAATATGTTTACATATGCATAGCGCTGCA | | | | |
| 59 | AGTTTATGGGCCAGAATATTCTGTATACCAG | RASL11B | ENSG00000128045 | NM_023940 | + |
| | ACATTGGTAAGCTCTCATGGTTTACAGGA | | | | |
| 60 | CCATGTGGCCAGTCTACCATGGGGCCCAGGA | GSDMD | ENSG00000278718 | NM_024736 | + |
| | GTTGGGGAAACACAATAAAGGTGGCATAC | | | | |
| 61 | ATGCTTAAACCCACGGAAGGGGGAGACTCTT | LAMP5 | ENSG00000125869 | NM_012261 | + |
| | TCGGATTTGTAGGGTGAAATGGCAATTAT | | | | |
| 62 | TTCTTTCTTCAAAGAGTCATCAGAATAACATG | CHPT1, SYCP3 | ENSG00000111666, | NM_153694 | + |
| | GATTGAAGAGACTTCCGAACACTTGCTA | | ENSG00000139351 | | |
| 63 | TGAAGTCAGCGTTAACCATGTGCATACAACT | ZNF627 | ENSG00000198551 | NM_145295 | + |
| | TAAGGAATTTTTTCCTCCTCATGTAAATT | | | | |
| 64 | GTTAAACAGGGATTATAGTACTTGTCTCACA | COL23A1 | ENSG00000050767 | NT_023133 | + |
| | AAGTTTCTGTGAGAATTAAACAAGGGGAT | | | | |
| 65 | CAGCCTGTGTGATACAAGTTTGATCCCAGGA | SCUBE2 | ENSG00000175356 | NM_020974 | + |
| | ACTTGAGTTCTAAGCAGTGCTCGTGAAAA | | | | |
| 66 | AATGCACAGATCTGCTTGATCAATTCCCTTGA | AC023024.1, | ENSG00000259172, | NM_138319 | + |
| | ATAGGGAAGTAACATTTGCCTTAAATTT | PCSK6 | ENSG00000140479 | | |
| 67 | TTTCCAATAACCACCTAAATTTTAACAAAGGT | RBP3 | ENSG00000265203 | NM_002900 | + |
| | TCCTTCTAAGTGGTAGAACTTGGGGTGG | | | | |
| 68 | AGTTATGCTTCCCTTCATGTTATATGCACATT | MYRIP, | ENSG00000170011, | NM_015460 | + |
| | GCCAAGAATTACTGTCAAGAGAAATGAT | EIF1B-AS1 | ENSG00000280739 | | |
| 69 | AAGGTTTGAAGGTTACGGCTCAGGGCTGCCC | SPEF1 | ENSG00000101222 | NM_015417 | + |
| | CATTAAAGTCAGTGTTGTGTTCTAAAAAA | | | | |
| 70 | GGACTGTATGAATTTATAGAAAATTGAATCTA | CLSTN2 | ENSG00000158258 | NT_005612 | + |
| | ATTTCAGAAGAGCGCACTGTCTTCTCAG | | | | |
| 71 | TACATTTCTTTGGGTTTCTAGAGACGCCCCTA | EVL, DEGS2 | ENSG00000196405, | NM_016337 | + |
| | AGTCACCTGCTTCATTAGACGGTTTCCA | | ENSG00000168350 | | |
| 72 | GGCCTAATTGAGGGAAGGAGGAAATTCATAC | ELMOD3 | ENSG00000115459 | NM_032213 | + |
| | CAGCAGTTTTCAAATAAAAGAATTGTTCT | | | | |
| 73 | TCCAATTCTACACTCAGTTAAAGACCATTACT | BBOF1, ALDH6A1 | ENSG00000119636, | NM_005589 | + |
| | TCTCAGTGGAAAGAAGAAGATGCTACTC | | ENSG00000119711 | | |
| 74 | GTGGGGACTTCGTGGGAGGCACTCATGGCTC | KIAA1683 | ENSG00000130518 | NM_025249 | + |
| | TCTGGGTCTAATGAATAAAGTCCTCCACA | | | | |
| 75 | CCAGGATCTTAAGGAAGAATATTCTAGGAAG | SPC25 | ENSG00000152253 | NM_020675 | − |
| | AAGGAAACTATTTCTACTGCTAATAAAGC | | | | |
| 76 | AGAAAACCCTTTTCTACAGTTAGGGTTGAGT | TFRC | ENSG00000072274 | NM_003234 | − |
| | TACTTCCTATCAAGCCAGTACGTGCTAAC | | | | |
| 77 | TAGGGAATGAATGAATGAATATGGATTGCTG | PAQR3 | ENSG00000163291 | NM 177453 | − |
| | TTAACTAGAAACACTTCTGTATGTCAGTC | | | | |
| 78 | GTACTTAGCTGGAAGAACATGTTAATTCTGC | MLLT10 | ENSG00000078403 | NM_1009569 | − |
| | AATATGTTTCTTGGTTAAACATTGCACAG | | | | |
| 79 | ACTCTCTTAGGTCATTTTTCAATGTGTGTAAC | CENPBD1 | ENSG00000177946 | NM_145039 | − |
| | CAAAAGTTAATCAGAATAAAGCGGAAGC | | | | |
| 80 | AATGCTTTGTTGGAGTTTAAAAATTCAGGGA | AL44926, GPSM2, | ENSG00000274068, | NM_013296 | − |
| | AAAAATCGGCAGACCATTAGTTACTATGG | CLCC1 | ENSG00000121957, | | |
| | | | ENSG00000121940 | | |
| 81 | AAGAAACCAGCATGTGACTTTCCTAGATAAC | PIMREG | ENSG00000129195 | NM_019013 | − |
| | ACTGCTTTCTCATAATAAAGACTATTTGC | | | | |
| 82 | GTTGGCATTGATATGGTACAACCTGCAAATT | HACD2 | ENSG00000206527 | NM_198402 | − |
| | ACTTGCAGTTCTGAGTTTCAGATAAAACA | | | | |
| 83 | AGTGTCATTTTAAGGGACATTTTTATGACTTT | ACE, | ENSG00000159640, | NM_152831 | − |
| | TATGTGTATGTTTATGTAGAAATTTGGA | AC113554 | ENSG00000264813 | | |
| 84 | ACTCACTTCTTTTCAGGTGTAGCTACAATTGT | OXCT1 | ENSG00000083720 | NM_000436 | − |
| | GTAATGTACAATATTAGAGAAAGGACAG | | | | |
| 85 | CCTGGGAGCAAATGAACAATAGCTAAGTGTC | GNAZ | ENSG00000128266 | NM_002073 | − |
| | TTGGTATTTAAAGAGTAAATTATTTGTGG | | | | |
| 86 | CCAAGAATATATGCTACAGATATAAGACAGA | FLT1 | ENSG00000102755 | NM_002019 | − |
| | CATGGTTTGGTCCTATATTTCTAGTCATG | | | | |
| 87 | ATGCTTTCCTAAATCAGATGTTTTGGTCAAGT | MAD2L1, MNAT1 | ENSG00000164109, | NM_002358 | − |
| | AGTTTGACTCAGTATAGGTAGGGAGATA | | ENSG00000020426 | | |
| 88 | ATTTGTGTGGACAAAAATATTTACACTTAGG | CDC25B | ENSG00000101224 | NM_004358 | − |
| | GTTTGGAGCTATTCAAGAGGAAATGTCAC | | | | |
| 89 | AAATATACTATGTTTGCGAACCTTGGTAGCTA | KIF21A | ENSG00000139116 | NM_017641 | − |
| | TGATGAGAGCTATTATCATCTGTGGTGG | | | | |
| 90 | TCAATGAAAGTTCAAGAACCTCCTGTACTTAA | HMGB3 | ENSG00000029993 | NM_005342 | − |
| | ACACGATTCGCAACGTTCTGTTATTTTT | | | | |
| 91 | ACCTTGATAGTTCACCACGTCTGATGGATCC | PTDSS1 | ENSG00000156471 | NM_014754 | − |
| | CTGTTTTAAATAAAAACGATTCACTTTAA | | | | |
| 92 | TAAAATACTTCAATCCTGGATTCACAGTGGG | MTMR2 | ENSG00000087053 | NM_016156 | − |
| | AACAAGTTTCTATTAAAAGGCAAATGCTG | | | | |
| 93 | GGCTGTGAACAATGTTAAATAGCATCAGTTT | CENPU | ENSG00000151725 | NM_024629 | − |
| | GTCCAATAGTTTTAAAGGCCATAATCATC | | | | |
| 94 | ACGAGTACCGGCATGTTATGTTACCCAGAGA | AL353705 | ENSG00000234819 | NM_001827 | − |
| | ACTTTCCAAACAAGTACCTAAAACTCATC | | | | |
| 95 | ATTTTTTAGAAAATACACACTTTTCAGGAGAA | Clorf198 | ENSG00000119280 | NM_032800 | − |
| | ACCTGAGCATGATTTTGGATTCTCCACC | | | | |
| 96 | CAGCTCAGACCATTTCCTAATCAGTTGAAAG | RRM2, | ENSG00000171848, | NM_001034 | − |
| | GGAAACAAGTATTTCAGTCTCAAAATTGA | AC007240 | ENSG00000284681 | | |
| 97 | CACTGCAGACTCTCAAGAGATCAATCAAATT | INTS7 | ENSG00000143493 | NM_015434 | − |
| | GCCAGAAACAGTTTGGTTTTTCATATGGA | | | | |
| 98 | TGAAACTTTCTTCTGATGAGTTTCTTTAACGT | MRPL13 | ENSG00000172172 | NM_014078 | − |
| | ACAGGATGGAGTAAAACAAATGGTACAG | | | | |
| 99 | CAATTCTTGAGAGTTAATGTGATCATGATATT | ARMC1 | ENSG00000104442 | NM_018120 | − |
| | GCAAACAACTATAAATGGTCTCTAGGCC | | | | |
| 100 | GAAGGAAACACCGAGTCTCTGTATAATCTAT | ADM | ENSG00000148926 | NM_001124 | − |
| | TTACATAAAATGGGTGATATGCGAACAGC | | | | |
| 101 | AGCAACCTGGGCCTTGTACTGTCTGTGTTTTT | IGFBP5 | ENSG00000115461 | NM_000599 | − |
| | AAAACCACTAAAGTGCAAGAATTACATT | | | | |
| 102 | GGGAATTTGATGCAGTAAAGATTACCCTGTT | SKA3 | ENSG00000165480 | NM_145061 | − |
| | TTATGATTGTTCCTTGAAAGTCAAATGGG | | | | |
| 103 | TAAGGCTAATGATACCAATGAGGGTTGGTTT | SLC7A1 | ENSG00000139514 | NM_003045 | − |
| | ATTATCAAACCTGAATAGCTGTGGTTTCT | | | | |
| 104 | TGGGGAGATACATCTTATAGAGTTAGAAATA | PRAME | ENSG00000185686 | NM_006115 | − |
| | GAATCTGAATTTCTAAAGGGAGATTCTGG | | | | |
| 105 | TATCTTGAAACTGACCAAACGCTTATTGTGTA | CTSV | ENSG00000136943 | NM_001333 | − |
| | AGATAAACCAGTTGAATCATTGAGGATC | | | | |
| 106 | TTCTCTGAAGGAATCATGTTCAGTGTTCGAC | SMC4P1 | ENSG00000229568, | NM_1002799 | − |
| | CACCTAAGAAAAGTTGGAAAAAGATCTTC | AC07959 | ENSG00000248710, | | |
| | | SMC4 | ENSG00000113810, | | |
| | | TRIM59 | ENSG00000213186 | | |
| 107 | TGTCATAGACATGTATTGGGGAGCTTCCAAT | NIPA1 | ENSG00000170113 | NM_144599 | − |
| | TAGCATACATAGACACATGTGTCAGTGGC | | | | |
| 108 | TGTCCATGCTACAAGAAGTTATGAGCCTTGT | SFT2D2 | ENSG00000213064 | NM_005149 | − |
| | TCTAAGTACAGATGAACCTTGTATTTGTG | | | | |
| 109 | ATCCCGATTTCAGTCAGACAAATACTCATTTC | SACS | ENSG00000151835 | NM_014363 | − |
| | AGAGATTCTATACTTCATGGAATCAAGA | | | | |
| 110 | AGTTACTTTCTTAATGTGACCTAGCAATAGGC | CTPS1 | ENSG00000171793 | NM_001905 | − |
| | ATAGCTACGTGGCACTATATTCTGGCCA | | | | |
| 111 | GAAATCTCTCTACACAGATGAGTCATCCAAA | NUSAP1 | ENSG00000137804 | NM_018454 | − |
| | CCTGGGAAAAAATAAAAGAACTGCAATCA | | | | |
| 112 | AAATTGCTAAGTGGAATGCATGAATTGCATT | PSMD7 | ENSG00000103035, | NM_002811 | − |
| | ATGTTCTCTGGTAACACGTAGAGTTCAGA | AC009120 | ENSG00000259972 | | |
| 113 | CCAAAGGTCTTGGTACAACCAGCTGCCCATT | BUD23 | ENSG00000071462, | NM_004603 | − |
| | TTGTGAAATTTTTATGTAGAATAAACATT | STX1A | ENSG00000106089 | | |
| 114 | GTTTCGGGTCTTTACCTCATAGTATGAAATTA | KIAA1147 | ENSG00000257093 | XM_1130020 | − |
| | GTAAGACACTGCATAGATTTTGCCCTGA | | | | |
| 115 | GAGTACGGATGGGAAACTATTGTGCACAAGT | NDRG1 | ENSG00000104419 | NM_006096 | − |
| | CTTTCCAGAGGAGTTTCTTAATGAGATAT | | | | |
| 116 | TATTTTATCAGCACTTTATGCACGTATTATTG | PFKP | ENSG00000067057, | NM_002627 | − |
| | ACATTAATACCTAATCGGCGAGTGCCCA | AL45116 | ENSG00000278419 | | |
| 117 | TGCCCTATGGAAAACTTGTCCAAATAACATTT | CD163L1 | ENSG00000177675 | NM 174941 | − |
| | CTTGAACAATAGGAGAACAGCTAAATTG | | | | |
| 118 | CTCCTTGTCATTGACCTTAGCTAAACCATGGC | MAPRE2 | ENSG00000166974 | NM_014268 | − |
| | AATTCATAAATAGAGGAAACATTAATGA | | | | |
| 119 | CTGAACGAGAACAAGAATCAGAAGAAGAAAT | TMEM45A | ENSG00000181458 | NM_018004 | − |
| | GTGACTTTGATGAGCTTCCAGTTTTTCTA | | | | |
| 120 | TATATTATCAGTCTGTACCAGTAGACCAGTAC | PABPC1 | ENSG00000070756 | NM_002568 | − |
| | CCTAACTACTGAAAAGAATATGGCAGTT | | | | |
| 121 | AGTAACGCTAACTTTGTACGGACGATGTCTC | RHBDF2 | ENSG00000129667 | NM_001005498 | − |
| | ATGGATTAAATAATATTCTTTATGGCAGT | | | | |
| 122 | GTGGATCTACCTCAGTTAAACAGTTGGGTGC | AGO2 | ENSG00000123908 | AF093097 | − |
| | TATTACTAAGTCTGTCAAATTAAATTGGA | | | | |
| 123 | CATTCTAAAGGGAAATCAGTAAAATGTCTTG | TMEM64 | ENSG00000180694 | NM_1008495 | − |
| | ATAATTGGTATCCAAATCACTTGTGTGCC | | | | |
| 124 | CCAAAGACAAACGATTAGAAGATGGCTATTT | MGAT4A | ENSG00000071073 | NM_012214 | − |
| | CAGAATAGGAAAATTTGAGAATGGTGTTG | | | | |
| 125 | CAAACTTCCTGACACTACTTCCATATTTGCAC | CDK16 | ENSG00000102225 | NM_006201 | − |
| | TAAAGGAGATTCAGCTACAAAAGGAGGC | | | | |
| 126 | ACCTTCCTATGAAGATCATGGAATCAAATAC | AL589666 | ENSG00000271793, | NM_006372 | − |
| | GGGACATTGAACTAATACTTGGACTTTGA | SYNCRIP | ENSG00000135316 | | |
| 127 | GGCTAACACAATCTAATTTTGGTTTAAGAGA | HIF1A | ENSG00000100644 | NM_181054 | − |
| | CAAATCTAGAGTCTCAAATGATCTCAGAG | | | | |
| 128 | TGGACCCTTAAATATGACTAAAATCACAGCA | RRAGD | ENSG00000025039 | NM_021244 | − |
| | ATATTGTTACATACGGGTTATATGCCAAC | | | | |
| 129 | TAAGCATTGTGAAGGAAGATTAATATAGCCA | HIF1A | ENSG00000100644 | NM 181054 | − |
| | AATAACTAGAGTGATCAGTTCTACCAGAG | | | | |
| 130 | CCTGGATAAAAGTACTGTATGATTTTGTGAT | DEGS1 | ENSG00000143753 | NM_144780 | − |
| | GGATGATACAATAAGTCCCTACTCAAGAA | | | | |
| 131 | GCTTTGTTACTTTGTTAGGTACGAATCACATA | LRP12 | ENSG00000147650 | NM_013437 | − |
| | AGGGAGATTGTATACAAGTTGGAGCAAT | | | | |
| 132 | TAAAAGATGAAGAAAGCTATTAGGTATATTT | ZDHHC20 | ENSG00000180776 | NT_024524 | − |
| | GTACATGACTGCAAATGAGTCTATGCCCG | | | | |
| 133 | GTGTGTTATCTTTATATGTCAAACTGGTTGAA | PLEKHA1 | ENSG00000107679 | NM_021622 | − |
| | CACTGTAATGAGAATAAACTGCACAGAG | | | | |
| 134 | GATTATTGTACGAAGTGTCTCTGTAATTATCA | FBXO5 | ENSG00000112029 | NM_012177 | − |
| | TACTACTAAAGACTGTTCAGATGGCAAG | | | | |
| 135 | CATTTGTATTAATGGAATACTAAGTCCCTCTG | NEAT1 | ENSG00000245532 | NT_033903 | − |
| | TGATTTCTGAACCAAGCTATTCCTAGGC | | | | |
| 136 | ATGAAGAGATTTCTCAAGCTATTCTTGATTTC | PIR | ENSG00000087842 | NM_003662 | − |
| | AGAAACGCAAAAAATGGGTTTGAAAGGG | | | | |
| 137 | AGCCAATCATGAGTACGTAAAGTGATTTTTG | ASPM | ENSG00000066279 | NM_018136 | − |
| | CTCTCTGTGTACAACTTTTAAAATCTGAC | | | | |
| 138 | ATCCTAGACCATATTTTCAAGTCATCTTAGCA | GBE1 | ENSG00000114480 | NM_000158 | − |
| | GCTAGGATTCTCAAATGGAAGTGTTATA | | | | |
| 139 | AGTGATTTCATGCTAGAAAAATTGGAAACTA | HJURP | ENSG00000123485 | NM_018410 | − |
| | AAAGTGTGTAGCTAGGTTATTTCGGAGTG | | | | |
| 140 | GCTAAGCCAAGTAGTAGCAGTAAAACTTCTG | QSER1 | ENSG00000060749 | NM_024774 | − |
| | ATCCTCTAGCATCAAAAACTACAACTACA | | | | |
| 141 | GGAAAGAAGTTGAAAGCATCTTGAAGAAAAA | BNIP3 | ENSG00000176171 | NM_004052 | − |
| | CTCAGATTGGATATGGGATTGGTCAAGTC | | | | |
| 142 | ACCTGGATATGTCTGTGAGGCTCCTGAAAGG | AC087521 | ENSG00000254409, | BC052560 | − |
| | AGACAAATAAAGTCAATATATTTGCACAA | C11orf96 | ENSG00000187479, | | |
| | | AC087521 | ENSG00000244953 | | |
| 143 | GGGTATGAAAGATGAGTGTCTGTAAAAATCC | LINC00888 | ENSG00000240024 | NT_005612 | − |
| | TTCTTAGAAATGTATTTCCTCAAGACTCT | | | | |
| 144 | CAGATGGCAAGATTGAGTTTATTTCAACAAT | GGH | ENSG00000137563 | NM_003878 | − |
| | GGAAGGATATAAGTATCCAGTATATGGTG | | | | |
| 145 | GAAACTGTGTCACCCTAAAGAAGCATATAAT | TRIP13 | ENSG00000071539 | NM_004237 | − |
| | CATAGCATTAAAAATGCACACATTACTCC | | | | |
| 146 | CAAGCGTGTTTCTAGAGAACAGTTGAGAGAG | STMN1 | ENSG00000117632 | NM_005563 | − |
| | AATCTCAAGATTCTACTTGGTGGTTTGCT | | | | |
| 147 | CCGACAAGAGGAGATCATTTTAGATATTACC | CENPN | ENSG00000166451, | NM_018455 | − |
| | GAAATGAAGAAAGCTTGCAATTAGTGAAC | AC092718 | ENSG00000260213, | | |
| | | AC092718 | ENSG00000284512 | | |
| 148 | TAATAGCAAAATTTAACCCGTTACTCTTTAAC | MYO10 | ENSG00000145555 | NM_012334 | − |
| | CTTGTACTGGAAATTCTAAGCAGTGCAG | | | | |
| 149 | CTTCCTACCTCTGGTGATGGTTTCCACAGGA | TK1 | ENSG00000167900 | NM_003258 | − |
| | ACAACAGCATCTTTCACCAAGATGGGTGG | | | | |
| 150 | AAATCATTCGGTAAATCCAAACTGCTATGCA | RUNX1 | ENSG00000159216, | NM_004456 | − |
| | AAAGTTATGATGGTTAACGGTGATCACAG | EZH2P1 | ENSG00000231300 | | |
| 151 | TTGGGTTTCTAGTCCTCCTTACCATCATCTCC | AURKA | ENSG00000087586 | NM_003600 | − |
| | ATATGAGAGTGTGAAAATAGGAACACGT | | | | |
| 152 | GCTGGTGGAGTAGCAGATGATATTAATACTA | DLGAP5 | ENSG00000126787 | NM_014750 | − |
| | ACAAAAAAGAAGGAATTTCAGATGTTGTG | | | | |
| 153 | TCACCCAGAACCAATGCGGTGTTTCTTAATG | TBCE | ENSG00000285053, | NM_152490 | − |
| | TTTGCACAAATTTCCTTAAAAATCAACTT | B3GALNT2 | ENSG00000162885 | | |
| 154 | CAGGACTTCTCTTTAGTCAGGGCATGCTTTAT | CENPF | ENSG00000117724 | NM_016343 | − |
| | TAGTGAGGAGAAAACAATTCCTTAGAAG | | | | |
| 155 | CCCTGTGCTATCGTAAGTTTGTTTTGAGCACT | AL117350 | ENSG00000237481, | NM_145257 | − |
| | GCATTCACTTTAAAATTCTGGAGGAACA | CCSAP | ENSG00000154429 | | |
| 156 | CAACATATTTCAGTTGGAAAATTTGTATGCAG | ATAD2 | ENSG00000156802 | NM_014109 | − |
| | TAATCAGCCAATGTATTTATCGGCATCG | | | | |
| 157 | CCCCCATTCTGGAAGGTTTTGTTATCTTCGGA | PSMD2 | ENSG00000175166, | NM_002808 | − |
| | AGAACCCCAATTATGATCTCTAAGTGAC | FMN2 | ENSG00000155816, | | |
| | | AL359918 | ENSG00000228818 | | |
| 158 | TGTCCCCAGGGATCAAACAGAAGCAGCCGTG | SHMT2 | ENSG00000182199, | NM_020142 | − |
| | GGCAAAATACAATTTCATTTAACAAATTG | NDUFA4L2 | ENSG00000185633 | | |
| 159 | AAACAGCATTATGGAGTTAAAAGATTTTTACA | PIMREG | ENSG00000129195, | NM_019013 | − |
| | ACTGGGTCTTGATTTTGATGTGAGCTGG | PITPNM3 | ENSG00000091622 | | |
| 160 | TCCAGACGCACTGATCTTTGCAAAGGAGACT | DCK | ENSG00000156136 | NM_000788 | − |
| | TAATTTCAAATCTGTAATTACCATACATA | | | | |
| 161 | CATTTGGCTGTCAGAAATTATACCGAGTCTA | DTL | ENSG00000143476 | NM_016448 | − |
| | CTGGGTATAACATGTCTCACTTGGAAAGC | | | | |
| 162 | TTAAAGGCAAAACTGTGCTCTTTATTTTAAAA | COL4A2 | ENSG00000134871 | NM_001846 | − |
| | AACACTGATAATCACACTGCGGTAGGTC | | | | |
| 163 | AAGGTGCTGTCATATATCTTGGAATGAATGA | AGFG1 | ENSG00000173744 | NM_004504 | − |
| | CCTAAAATCATTTTAACCATTGCTACTGG | | | | |
| 164 | GGATGTAAATCCTGAGCTCAAATCTCTGTTA | NMB | ENSG00000197696 | NM_205858 | − |
| | CTCCATTACTGTGATTTCTGGCTGGGTCA | | | | |
| 165 | CCTCAAGAGTATGTATAATTTGAAGAGATAC | KIF14 | ENSG00000118193 | NM_014875 | − |
| | TTTGTAACTATGCTTGGGTGATATTGAGC | | | | |
| 166 | TTCACAGAATAGCACAAACTACAATTAAAACT | BIRC5 | ENSG00000089685 | NM_001012271 | − |
| | AAGCACAAAGCCATTCTAAGTCATTGGG | | | | |
| 167 | CCAGCACATAGGAGAGATGAGCTTCCTACAG | VEGFA | ENSG00000112715 | NM_003376 | − |
| | CACAACAAATGTGAATGCAGACCAAAGAA | | | | |
| 168 | GAGAAACATTGTATATTTTGCAAAAACAAGA | ECT2 | ENSG00000114346 | NM_018098 | − |
| | TGTTTGTAGCTGTTTCAGAGAGAGTACGG | | | | |
| 169 | TACTTTTTGGAAAAGAATAAACCAAGAATTG | IVNS1ABP | ENSG00000116679 | NM_016389 | − |
| | ATTGGGCACATCATTTCAAGAAGTCCCTC | | | | |
| 170 | ATGGAGTTGCTAGTAAAGCGAAGCTGATTAT | MCCC1 | ENSG00000078070 | NM_020166 | − |
| | CCTGGAAAACACTATTTACCTATTTTCCA | | | | |
| 171 | GACTGCTAGTGGATAATAACATCTTGACTAC | TMEM38B | ENSG00000095209, | NM_017779 | − |
| | TTAAAAAAGGGACATATTGAAAATCCTGG | AL592437 | ENSG00000232486, | | |
| | | OTUD7A | ENSG00000169918, | | |
| | | AC026951 | ENSG00000259358, | | |
| | | DEPDC1 | ENSG00000024526, | | |
| | | AL138789 | ENSG00000233589 | | |
| 172 | CATGTTACCTGGACTGGAACAGACTGTGAAT | INAVA | ENSG00000163362, | NM_018265 | − |
| | ATAGCAGAAGGTTCCAAGAACTCTGGTGT | SLC9C1 | ENSG00000172139 | | |
| 173 | GAGACCAGGTGCTTCAAAACTTAGGCTCGGT | KIF21A | ENSG00000139116 | NM_017641 | − |
| | AGAATCTTACTCAGAAGAAAAAGCAAAAA | | | | |
| 174 | GGATTCAACCCAAATGATTTCTCATCAGGTG | C16orf95 | ENSG00000260456 | AK026130 | − |
| | ATTCTTGGTTGTAGCAAAGTTCATGTGAA | | | | |
| 175 | AGAACTCTTGATTTTGTACATAGTCCTCTGGT | CCNB2 | ENSG00000157456, | NM_004701 | − |
| | CTATCTCATGAAACCTCTTCTCAGACCA | AC092757 | ENSG00000259732 | | |
| 176 | AATTGGTAAACATCATGTTCCTGATGATAACC | STK3 | ENSG00000104375 | NM_006281 | |
| | CAGTAGCAAAAACATTTGTACTGAGTGG | | | | |
| 177 | CATCAGTCTTGGGAAATTTGAACTTTGATCAA | ZNF367 | ENSG00000165244 | NM_153695 | |
| | CTTAACTAAAGAAGGAAGGGTAGTAAGA | | | | |
| 178 | TTAGGGCCCTACGTAATAGGCTAATTGTACT | BUB1 | ENSG00000169679 | NM_004336 | − |
| | GCTCTTAGAATGTAAGCGTTCACGAAAAT | | | | |
| 179 | GAGTCTTTGGGATACCATTAAAAAGAAGAAA | ASPM | ENSG00000066279 | NM_018136 | − |
| | ATTTCAGCCTCTACAAGTCACAACAGAAG | | | | |
| 180 | AGAGTGTGAAAAATAGGAACACGTGCTCTAC | AURKA | ENSG00000087586 | NM_003600 | − |
| | CTCCATTTAGGGATTTGCTTGGGATACAG | | | | |
| 181 | AACTTTTTAGGGCAAAGTTAACACTGAAAGT | UTP23 | ENSG00000147679, | NM_006265 | − |
| | TCTAGCTTAAGTGTTGAAACTTTTGTGGG | RAD21 | ENSG00000164754 | | |
| 182 | ACTTAGCATTTTCTGCATCTCCACTTGGCATT | PGK1 | ENSG00000102144, | NM_000291 | − |
| | AGCTAAAACCTTCCATGTCAAGATTCAG | OPHN1 | ENSG0000079482, | | |
| | | AC010422 | ENSG00000269693 | | |
| 183 | TTTTGATGAGAATGAATCTTGGTACTTAGATG | CP | ENSG00000047457, | NM_000096 | − |
| | ACAACATCAAAACATACTCTGATCACCC | LRRC69 | ENSG00000214954, | | |
| | | AC104966 | ENSG00000253525 | | |
| 184 | TTCCCTTCAATACTCCTAAAACCAAAGAAGG | AC079781 | ENSG00000284707, | NM_183356 | − |
| | ATATTACTACCGTCAAGTCTTTGAACGCC | ASNS | ENSG00000070669 | | |
| 185 | TCCTGTCCTGCTCATTATGCCACTTCCTTTTA | CA9 | ENSG00000107159 | NM_001216 | − |
| | ACTGCCAAGAAATTTTTTAAAATAAATA | | | | |
| 186 | CAAAAACTCAGATCTATCTTAAGAGTGACCA | AL451164 | ENSG00000278419, | NM_014889 | − |
| | GGAAGAGGTTCATTGAAATAATCATGCAT | PITRM1 | ENSG00000107959 | | |
| 187 | CATACGGTTTTGTTTGGAGGATGGCTTCTGC | TMEM74B | ENSG00000125895 | NM_018354 | − |
| | TGCTAAAAATACAAAAGTTTGGAAACCGC | | | | |
| 188 | CAGAGGGACCTTATTTAAACATAAGTGCTGT | ESM1 | ENSG00000164283 | NM_007036 | − |
| | GACTTCGGTGAATTTTCAATTTAAGGTAT | | | | |
| 189 | GTTTGTGAAACTGTTAAGGTCCTTTCTAAATT | CCNE2 | ENSG00000175305 | NM_057749 | − |
| | CCTCCATTGTGAGATAAGGACAGTGTCA | | | | |
| 190 | TTAACCAGCTGTAAAACACAGACCTTTATCAA | EGLN1 | ENSG00000135766 | NM_022051 | − |
| | GAGTAGGCAAAGATTTTCAGGATTCATA | | | | |
| 191 | GGGGATGAATAGAAAACCTGTAAGCTTTGAT | CENPA | ENSG00000115163 | NM_001809 | − |
| | GTTCTGGTTACTTCTAGTAAATTCCTGTC | | | | |
| 192 | GTGATAAAGTACCTGATCCAAATGTTATGAG | LIN9 | ENSG00000183814 | NT_004559 | − |
| | AATACTGGACGAGAATTGAACGAAATTGA | | | | |
| 193 | TGCAGCAGTACTACTGTCAACATAGTGTAAA | PRC1 | ENSG00000198901 | NM 199413 | − |
| | TGGTTCTCAAAAGCTTACCAGTGTGGACT | | | | |
| 194 | GCATGAGTCACAATTACAAAGTTTTGAGCGG | PALM2-AKAP2 | ENSG00000157654; | NM_147150 | − |
| | TTTTGTAATTTGACATTTAGGAAAGTCTC | AKAP2 | ENSG00000241978 | | |
| 195 | TTATTCGAAGACACAGAAGTTGGGCAAGTCA | NMU | ENSG00000109255 | NM_006681 | − |
| | AATGTTGTGTCGTCAGTTGTGCATCCGTT | | | | |
| 196 | TGTACTGGCAGGCTCGTTTTACCTGATTCTA | PITRM1 | ENSG00000107959 | NM_014889 | − |
| | GAATATTTAAGAATCTAAAAATAAAGGGC | | | | |
| 197 | GTGGCCTATAACTTACTTGTCAACAACTGTG | HRASLS | ENSG00000127252 | NM_020386 | − |
| | AACATTTTGTGACATTGCTTCGCTATGGA | | | | |
| 198 | CCAGGACGCCACTCATTTCATCTCATTTAAG | IGFBP5 | ENSG00000115461 | NM_000599 | − |
| | GGAAAAATATATATCTATCTATTTGAGGA | | | | |
| 199 | CGGAGCGCAGGGTACTTGGCGTATAATAAGC | JHDM1D-AS1 | ENSG00000260231 | NT_007914 | − |
| | CATCAATAATTTATGGGTGAAATTGAGAG | | | | |
| 200 | CAGAGCTACAACTAGGAAAATTAGAGTGGTA | MSANTD3 | ENSG00000066697 | NM_080655 | − |
| | GTAGTCACTTATTTAAGAATTCATTCAGG | | | | |
| 201 | TTGGTAGTTAACCCTAACTACTTGCTCGAAG | MCM6 | ENSG00000076003 | NM_005915 | − |
| | ATTGAGATAGTGAAAGTAACTGACCAGAG | | | | |
| 202 | GCGTGAGCATGTCAGTATTCTAGTCCAGTAT | SMIM5 | ENSG00000204323 | XM_946181 | − |
| | TTGCCAGTTTCCAAGTAAAAGCTTTTGTG | | | | |
| 203 | GCTGTGCCATTCAATGTTTGATGCATAATTG | CDCA7 | ENSG00000144354 | NM_031942 | − |
| | GACCTTGAATCGATAAGTGTAAATACAGC | | | | |
| 204 | CCAAGAAGGAAAATGTCAAAATTAGTGATGA | RFC4 | ENSG00000163918 | NM_181573 | − |
| | GGGAATAGCTTATCTTGTTAAAGTGTCAG | | | | |
| 205 | TGCTTTAAGTGAATGGCAGTCCCTTGTCTTAT | ORC6 | ENSG00000091651 | NM_014321 | − |
| | TCAGAATATAAAATTCAGTCTGAATGGC | | | | |
| 206 | AGGTTGGCAGTAAGGCAGGGTCCCATTTCTC | SLC2A3 | ENSG00000059804 | NM_006931 | − |
| | ACTGAGAAGATTGTGAATATTTCCATATG | | | | |
| 207 | GTGCAAATAGAATTAGCAGTAAGAAGCTACT | ADGRG6 | ENSG00000112414 | NM_1032395 | − |
| | CTAGCTAATTTGCCATTTCACTTAAATGG | | | | |
| 208 | GATACAGCCTACATAAAGACTGTTATGATCG | MELK | ENSG00000165304 | NM_014791 | − |
| | CTTTGATTTTAAAGTTCATTGGAACTACC | | | | |
| 209 | CAACATTTACATTGTAATTCAATAGACGCTAC | GRHL2 | ENSG00000083307 | NT_008046 | − |
| | TACTACAAAGGAGCTTTATTCTTCCAGC | | | | |
| 210 | CAACAGTATTGCGTTGTCAGACTAGGAAAGC | MTDH | ENSG00000147649 | NM_178812 | − |
| | TAAACGAACAAAATGGTTTTAGTTTTGCT | | | | |
| 211 | CTGGTTGTCCAACTACCATATGAAGCTAGAA | UCHL5 | ENSG00000116750 | NM_015984 | − |
| | AATGCACAAACGATATTCCTTATCTGTAA | | | | |
| 212 | GGCATCAGGGATCACATCACTCTTAACGGCT | RAB6B | ENSG00000154917 | NM_016577 | − |
| | GTTACTTAAACAACTATTTTTTGGTTTGG | | | | |
| 213 | TGAAAATGTATTTGTAGTCACGGACTTTCAG | ECT2 | ENSG00000114346 | NM_018098 | − |
| | GATTCTGTCTTTAATGACCTCTACAAGGC | | | | |
| 214 | AGACCAGGTCTCTATTTTGAGGAAGAAATAC | EXT1 | ENSG00000182197 | NM_000127 | − |
| | CGAGACATTGAGCGACTTTGAGGAATCCG | | | | |
| 215 | AAGTCATGACACAGTATTCGCTCTTTTTCTGA | GPR180 | ENSG00000152749 | NM_180989 | − |
| | ATGTTTACATAGAGATTCATCACTGCAG | | | | |
| 216 | CAGTAAGTACGGGAAAAAATGTTTACTAACT | LPCAT1 | ENSG00000153395 | NM_024830 | − |
| | TCCTCAGAGATTCGTGATACGCGTTTCTC | | | | |
| 217 | CTTTGAATGGACATAAAAATTCTGCTTGTTAA | SERF1A | ENSG00000172058 | NM_021967 | − |
| | GAACAAGTTGAGCTCTGGTAACTGATCT | | | | |
| 218 | TGACTGATGTGTCTGAAAATGCTAAGGATCT | CDC42BPA | ENSG00000143776 | NM_003607 | − |
| | TATTCGAAGGCTCATTTGTAGCAGAGAAC | | | | |
| 219 | CTCTGAAAGAAGAAGTTCAAAAGCTGGATGA | NDC80 | ENSG00000080986 | NM_006101 | − |
| | TCTTTACCAACAAAAAATTAAGGAAGCAG | | | | |
| 220 | ATCTGTGGTTATTCGAACCTTTATTACTAGTG | GMPS | ENSG00000163655 | NM_003875 | − |
| | ACTTCATGACTGGTATACCTGCAACACC | | | | |
| 221 | TCCACCCCAGGACGCCACTCATTTCATCTCAT | IGFBP5 | ENSG00000115461 | NM_000599 | − |
| | TTAAGGGAAAAATATATATCTATCTATT | | | | |
| 222 | GGCCCTCTCTTCTCACCTTTGTTTTTTGTTGG | MMP9 | ENSG00000100985 | NM_004994 | − |
| | AGTGTTTCTAATAAACTTGGATTCTCTA | | | | |
| 223 | CTGGGTTGATACCTGAAAGAATCCTGTCTTA | CMC2 | ENSG00000103121 | NM_020188 | − |
| | TTTGGTCTCCATAATCCTTTGAATGGAAA | | | | |
| 224 | AGTACCCTGATATACTGAATTTTGTGGATGAT | DIAPH3 | ENSG00000139734 | NM_030932 | − |
| | TTGGAACCTTTAGACAAAGCTAGTAAAG | | | | |
| 225 | AAGACTTTCTTACTGACCTGAATAACTTCAGA | DIAPH3 | ENSG00000139734 | NM_030932 | − |
| | ACCACATTCATGCAAGCAATAAAGGAGA | | | | |
| 226 | TTTAGTGGTCCGTTGCCTCTGAAGATGTAAA | QSOX2 | ENSG00000165661 | NM_181701 | − |
| | CAAACAAATACACTATTTCTGGGAACATT | | | | |
| 227 | ATAGAATATGTATATGTATTCTTTGTCTACCA | TMEM65 | ENSG00000164983 | NT_008046 | − |
| | ACTACCAAAGAAACAAATACTCCTCAGT | | | | |
| 228 | ACATTGCTTACTTAAAAGCTACATAGCCCTAT | NUSAP1 | ENSG00000137804 | NM_018454 | − |
| | CGAAATGCGAGGATTAATGCTTTAATGC | | | | |
| 229 | ACCATAAGGCAATTGAGCACATAACGAAAAA | DIAPH3 | ENSG00000139734 | NM_001042517 | − |
| | TGATGCAATAAGAATGTATGCACTCTCTT | | | | |
| 230 | CAGCCTTTCCTCATGTCAACACAGTTCACAAT | MIR210HG | ENSG00000247095 | NT_035113 | − |
| | ATAGTTTTCAAAGTACAGTTTAAAACTC | | | | |
| 231 | CCTCCCCAAAATAATTAGTAACTGGTTGTTCT | TSPYL5 | ENSG00000180543 | NM_033512 | − |
| | ACTTGGTAATTTGACACCCTGTTAATAA | | | | |

TABLE 4
Overview of BluePrint ® genes

| | | | | | |
|---|---|---|---|---|---|
| NM_000663 | ABAT | NM_006864 | LILRB3 | NM_145186 | ABCC11 |
| NM_015541 | LRIG1 | NM_001609 | ACADSB | NM_001030002 | GRB7 |
| NM_024722 | ACBD4 | NM_005375 | MYB | NM_002286 | AFF3 |
| NM_001124 | ADM | NM_000662 | NATI | NM_006408 | AGR2 |
| NM_000909 | NPY1R | NM_000044 | AR | NM_153694 | SYCP3 |
| NM_000633 | BCL2 | NM_007083 | NUDT6 | NM_206925 | CA12 |
| NM_003766 | BECN1 | NM_017830 | OCIAD1 | NM_144575 | CAPN13 |
| NM_000060 | BTD | NM_032521 | PARD6B | NM_031942 | CDCA7 |
| NM_003939 | BTRC | NM_000926 | PGR | NM_001267 | CHAD |
| NM_203453 | PPAPDC2 | NM_005794 | DHRS2 | NM_006113 | VAV3 |
| NM_207310 | CCDC74B | NM_020820 | PREX1 | NM_000125 | ESR1 |
| NM_004358 | CDC25B | NM_032918 | RERG | NM_004496 | FOXA1 |
| NM_014246 | CELSR1 | NM_173079 | RUNDC1 | NM_001453 | FOXC1 |
| NM_001408 | CELSR2 | NM_002964 | S100A8 | NM_004448 | ERBB2 |
| NM_020974 | SCUBE2 | NM_006733 | KIF20A | NM_005080 | XBP1 |
| NM_016138 | COQ7 | NM_003108 | SOX11 | NM_019600 | KIAA1370 |
| NM_003462 | DNALI1 | NM_145006 | SUSD3 | NM_177433 | MAGED2 |
| NM_021814 | ELOVLS | NM_153365 | TAPT1 | NM_024101 | MLPH |
| NM_015130 | TBC1D9 | NM_020444 | MSN | NM_033426 | KIAA1737 |
| NM_001002295 | GATA3 | NM_024549 | TCTN1 | NM_018728 | MYO5C |
| NM_017786 | GOLSYN | NM_024817 | THSD4 | NM_033419 | PERLD1 |
| NM_014668 | GREB1 | NM_144686 | TMC4 | NM_175887 | PARIS |
| NM_024827 | HDAC11 | NM_032376 | TMEM101 | NM_138393 | REEP6 |
| NM_002115 | HK3 | NM_021103 | TMSB10 | NM_178568 | RTN4RL1 |
| NM_000191 | HMGCL | NM_198485 | TPRG1 | NM_004694 | SLC16A6 |
| NM_002184 | IL6ST | NM_152376 | UBXD3 | NM_015417 | SPEF1 |
| NM_005544 | IRS1 | NM_018478 | DBNDD2 | | |

What is claimed is:

1. A method of selecting a therapeutic treatment for a high-risk HER2+ or HER2− Stage II or Stage III breast cancer that is hormone receptor+ or hormone receptor−, the method comprising:
classifying the Stage II or Stage III breast cancer as having a positive or negative immune response profile for responding to an immunotherapy treatment, wherein a positive immune response profile is assigned by determining that the expression pattern of at least one panel of immune status genes reaches or exceeds a threshold that is associated with a high pathology complete response (pCR) rate for patients treated with an immune pathway-targeted therapy compared to patients treated with therapies that do not target the immune response; and a negative immune response profile is assigned by determining that the expression pattern is lower than the threshold;
classifying the Stage II or Stage III breast cancer as having a positive or negative DNA Repair Defect (DRD) profile for responding to a DNA repair treatment, wherein a positive DRD response profile is assigned by determining that the expression pattern of at least one panel of DRD status reaches or exceeds a threshold that is associated with a high pathology complete response (pCR) rate for patients treated with a DNA repair-targeted therapy compared to patients treated with therapies that do not target DNA repair; and a negative DRD response profile is assigned by determining that the expression pattern is lower than the threshold; and assigning the breast cancer to a treatment subtype selected from the group consisting of HER2−/Immune−/DRD−, HER2−/Immune−/DRD+, HER2−/Immune+, HER2+/% BP-HER2-type or Basal-type, and HER2+/BP-Luminal-type.

2. The method of claim 1, wherein classifying the Stage II or Stage III breast cancer as having a positive or negative immune response profile comprises evaluating expression levels of at least one panel of immune status genes, and wherein the panel is selected from a TcellBcell biomarker panel, a dendritic biomarker panel, a chemokine biomarker panel, a MastCell biomarker panel, a STAT1 biomarker panel, and a B-cell biomarker panel as set forth in Table B.

3. The method of claim 1, wherein the breast cancer is hormone receptor-positive (HR+).

4. The method of claim 3, wherein the breast cancer is HER2−.

5. The method of claim 4, wherein classifying the Stage II or Stage III breast cancer as having a positive or negative immune response profile comprises evaluating expression levels of B-cell and Mast-cell biomarker panels.

6. The method of claim 1, wherein the breast cancer is estrogen receptor-negative, progesterone receptor-negative and HER2-negative (triple negative).

7. The method of claim 6, wherein classifying the Stage II or Stage III breast cancer as having a positive or negative immune response profile comprises evaluating expression levels of a dendritic cell panel and a STAT1 and/or chemokine panel.

8. The method of claim 6, wherein classifying the breast cancer as having a positive DRD profile comprises determining that the expression pattern of a VCpred_TN gene panel set forth in Table B falls within a range that is associated with a high pCR rate for patients treated with a therapeutic agent that targets DNA repair compared to patients treated with a therapy that does not target DNA repair.

9. The method of claim 1, wherein classifying the Stage II or Stage III breast cancer as having a positive DRD response profile comprises evaluating expression levels of a PARPi7 or PARPi7_plus_MP2 panel.

10. The method of claim 1, wherein Stage II breast cancer is classified as a high-risk HER2+ breast cancer by MammaPrint® analysis.

11. The method of claim 1, further comprising selecting a DNA repair targeted therapy for a patient having a breast cancer assigned to the HER2−/Immune//DRD+ subtype, selecting an immune response therapy for a patient having a breast cancer assigned to the HER2−/Immune+ subtype: selecting a dual-anti-HER2 therapy for a patient assigned to the HER2+ that are not luminal subtype; selecting a combination therapy that comprises an AKT pathway-inhbitor for a patient assigned to the HER2+/BP-Luminal subtypes; and selecting neoadjuvant endocrine therapy for a patient assigned to the HER2−/Immune−/DRD− subtype.

12. The method of claim 11, wherein the immune response therapy is an PDL1/PD1 checkpoint inhibitor therapy, the DNA repair therapy is a platinum based therapy or PARP inhibitor; and the AKT pathway inhibitor is an AKT inhibitor.
